(19) United States
(12) Patent Application Publication
(10) Pub. No.: US 2021/0065842 A1
(43) Pub. Date: Mar. 4, 2021

(54) SYSTEMS AND METHODS FOR DETERMINING TUMOR FRACTION

(71) Applicant: GRAIL, Inc., Menlo Park, CA (US)

(72) Inventors: Anton Valouev, Palo Alto, CA (US); Jing Xiang, San Carlos, CA (US)

(21) Appl. No.: 16/936,901

(22) Filed: Jul. 23, 2020

Related U.S. Application Data
(60) Provisional application No. 62/877,755, filed on Jul. 23, 2019.

(51) Int. Cl. G16B 5/30; G16B 30/10; G16B 20/10; G16B 20/20; G06F 30/27

(52) U.S. Cl. CPC G16B 5/30; G16B 30/10; G06F 30/27; G16B 20/20; G16B 20/10

(57) ABSTRACT

Systems and methods for determining a tumor fraction for a subject are provided. A plurality of bin values is obtained. Each respective bin value in the plurality of bin values corresponds to a bin in a plurality of bins. Each bin represents a corresponding region of a reference genome. The plurality of bin values is derived from a first biological sample of the subject. A plurality of copy number values is determined at least in part from the plurality of bins values. A plurality of allele frequencies for a plurality of alleles is derived from a second biological sample of the subject. At least the plurality of copy number values and the plurality of allele frequencies, or a plurality of features derived therefrom, are applied to a reference model, thereby determining the tumor fraction of the subject.

### CROSS REFERENCE TO RELATED APPLICATION

This application claims priority to U.S. Provisional Patent Application No. 62/877,755 entitled “SYSTEMS AND METHODS FOR DETERMINING TUMOR FRACTION,” filed Jul. 23, 2019, which is hereby incorporated by reference.

### TECHNICAL FIELD

This specification describes using a reference model to determine tumor fraction of a subject.

### BACKGROUND

The increasing knowledge of the molecular basis for cancer and the rapid development of next generation sequencing techniques are advancing the study of early molecular alterations involved in cancer development in body fluids. Large-scale sequencing technologies, such as next generation sequencing (NGS), have afforded the opportunity to achieve sequencing at costs that are less than one U.S. dollar per million bases, and costs of less than ten U.S. cents per million bases have been realized. Specific genetic and epigenetic alterations associated with such cancer development are found in plasma, serum, and urine cell-free DNA (cfDNA). Such alterations could potentially be used as diagnostic biomarkers for several classes of cancers (see, Salvi et al., 2016, Onco Targets Ther. 9:6549-6559).

Cell-free DNA (cfDNA) can be found in serum, plasma, urine, and other body fluids (Chan et al., 2003, Ann Clin Biochem. 40(Pt 2):122-130) representing a “liquid biopsy,” which is a circulating picture of a specific disease (see, De Mattos-Arruda and Caldas, 2016, Mol Oncol. 10(3):464-474). This represents a potential, non-invasive method of screening for a variety of cancers.

The existence of cfDNA was demonstrated by Mandel and Metais decades ago (Mandel and Metais, 1948, C R Seances Soc Biol Fil. 142(3-4):241-243). cfDNA originates from necrotic or apoptotic cells, and it is generally released by all types of cells. Stroun et al. further showed that specific cancer alterations could be found in the cfDNA of patients (see, Stroun et al., 1989 Oncology 1989 46(5):318-322). A number of subsequent articles confirmed that cfDNA contains specific tumor-related alterations, such as mutations, methylation, and copy number variations (CNVs), thus confirming the existence of circulating tumor DNA (ctDNA) (see Goessl et al., 2000 Cancer Res. 60(21):5941-5945 and Frenel et al., 2015, Clin Cancer Res. 21(20):4586-4596).

cfDNA in plasma or serum is well characterized, while urine cfDNA (ucfDNA) has been traditionally less characterized. However, recent studies demonstrated that ucfDNA could also be a promising source of biomarkers (e.g., Casadio et al., 2013, Urol Oncol. 31(8):1744-1750).

In blood, apoptosis is a frequent event that determines the amount of cfDNA. In cancer patients, however, the amount of cfDNA seems to be also influenced by necrosis (see, Hao et al., 2014, Br J Cancer 111(8):1482-1489 and Zonta et al., 2015 Adv Clin Chem. 70:197-246). Since apoptosis seems to be the main release mechanism circulating cfDNA has a size distribution that reveals an enrichment in short fragments of about 167 bp, (see, Heitzer et al., 2015, Clin Chem. 61(1):112-123 and Lo et al., 2010, Sci Transl Med. 2(61):61ra91) corresponding to nucleosomes generated by apoptotic cells.

The amount of circulating cfDNA in serum and plasma seems to be significantly higher in patients with tumors than in healthy controls, especially in those with advanced-stage tumors than in early-stage tumors (see Sozzi et al., 2003, J Clin Oncol. 21(21):3902-3908, Kim et al., 2014, Ann Surg Treat Res. 86(3):136-142; and Shao et al., 2015, Oncol Lett. 10(6):3478-3482). The variability of the amount of circulating cfDNA is higher in cancer patients than in healthy individuals, (see, Heitzer et al., 2013, Int J Cancer. 133(2):346-356) and the amount of circulating cfDNA is influenced by several physiological and pathological conditions, including proinflammatory diseases (see, Raptis and Menard, 1980, J Clin Invest. 66(6):1391-1399, and Shapiro et al., 1983, Cancer 51(11):2116-2120).

Methylation status and other epigenetic modifications are known to be correlated with the presence of some cancer of origins such as cancer (see, Jones, 2002, Oncogene 21:5358-5360). In addition, specific patterns of methylation have been determined to be associated with particular cancer conditions (see Paska and Hudler, 2015, Biochemia Medica 25(2):161-176). Warton and Samimi have demonstrated that methylation patterns can be observed even in cell-free DNA (2015, Front Mol Biosci, 2(13) doi: 10.3389/fmolb.2015.00013).

Given the promise of circulating cfDNA, as well as other forms of genotypic data, as a diagnostic indicator, improved ways of assessing such data to identify tumor fraction in subjects are needed in the art.

### SUMMARY

The present disclosure addresses the shortcomings identified in the background by providing robust techniques for determining tumor fraction in subjects.

### A. Embodiments for Determining Tumor Fraction of a Subject

One aspect of the present disclosure provides a method of determining a tumor fraction for a subject of a species. The method is performed at a computer system comprising at least one processor and a memory storing at least one program for execution by the at least one processor. The at least one program executes the method and comprises instructions for obtaining, in electronic form, a first dataset that comprises a plurality of bin values. Each respective bin value in the plurality of bin values is for a corresponding bin in a plurality of bins. Each respective bin in the plurality of bins represents a corresponding region of a reference genome of the species. The plurality of bin values is derived from alignment of a first plurality of sequence reads, determined by a first nucleic acid sequencing of a first plurality of cell-free nucleic acids in a first biological sample, to a reference genome of the species. The first biological sample comprises a liquid sample of the subject and the first plurality of cell-free nucleic acids comprises at least 1000 cell-free nucleic acids.

Further in the method, a plurality of copy number values is determined at least in part from the plurality of bins values.

Further in the method, there is obtained, in electronic form, a second dataset that comprises a plurality of allele frequencies for a plurality of alleles. The plurality of allele frequencies is derived from alignment of a second plurality of sequence reads, determined by a second nucleic acid sequencing of a second plurality of cell-free nucleic acids in a second biological sample, to the reference genome. The second biological sample comprises a liquid sample of the subject and the second plurality of cell-free nucleic acids comprises at least 1000 cell-free nucleic acids.

Further in the method, there is applied, to a reference model, at least the plurality of copy number values and the plurality of allele frequencies, or a plurality of features derived therefrom, thereby determining the tumor fraction of the subject.

In some embodiments, the first and second datasets are separate data structures. In some alternative embodiments, the first and second datasets are in a single data structure.

In some embodiments, the first biological sample and the second biological sample are a single biological sample, the first nucleic acid sequencing and the second nucleic acid sequencing is the same nucleic acid sequencing, and the first plurality of cell-free nucleic acids and the second plurality of cell-free nucleic acids is a single plurality of cell-free nucleic acids.

In some embodiments, the first and second nucleic acid sequencing is targeted panel sequencing that provides both the plurality of bin values and the plurality of allele frequencies. Further, the targeted panel sequencing uses a plurality of probes. In such embodiments, each probe in the plurality of probes includes a nucleic acid sequence that corresponds to the sequence, or a complementary sequence thereof, of a portion of the reference genome represented by a corresponding one or more bins in the plurality of bins.

In some embodiments, the first nucleic acid sequencing is whole genome sequencing, and the second nucleic acid sequencing is targeted panel sequencing that uses a plurality of probes. In such embodiments, each probe in the plurality of probes includes a nucleic acid sequence that corresponds to the sequence, or a complementary sequence thereof, of a portion of the reference genome represented by a corresponding one or more bins in the plurality of bins.

In some embodiments, the second nucleic acid sequencing is a second targeted panel sequencing, and the second targeted panel sequencing uses a plurality of probes. In such embodiments, each probe in the plurality of probes includes a nucleic acid sequence that corresponds to the sequence, or a complementary sequence thereof, of an allele in the plurality of alleles.

In some embodiments that make use of a plurality of probes, a respective probe in the plurality of probes maps to a portion of the reference genome but has a respective nucleic acid sequence that varies with respect to the portion of the reference genome by one or more transitions, and each respective transition in the one or more transitions occurs at a respective un-methylated CpG dinucleotide site in the respective portion of the reference genome.

In some embodiments that make use of a plurality of probes, a respective probe in the plurality of probes maps to a portion of the reference genome but has a respective nucleic acid sequence that varies with respect to the portion of the reference genome by one or more transitions, and each respective transition in the one or more transitions occurs at a respective methylated CpG dinucleotide site in the respective portion of the reference genome.

In some embodiments, the method further comprising subjecting the cell-free nucleic acids of the first and second biological samples to a conversion treatment, prior to nucleic acid sequencing.

In some embodiments that make use of a plurality of probes, the plurality of probes comprises at least 5, at least 10, at least 25, at least 50, at least 100, at least 200, at least 300, at least 400, at least 500, at least 600, at least 700, at least 800, at least 900, at least 1000, or at least 3,000 probes.

In some embodiments, the deriving the plurality of bin values further comprises using the first plurality of sequence reads to determine a respective number of cell-free nucleic acids represented by the plurality of sequence reads that map to each respective bin in the plurality of bins.

In some embodiments, the method further comprises normalizing the plurality of bin values.

In some embodiments, each bin in the plurality of bins comprises at least 100 nucleic acid residues, at least 500 nucleic acid residues, at least 1000 nucleic acid residues, at least 2500 nucleic acid residues, at least 5000 nucleic acid residues, at least 10,000 nucleic acid residues, at least 25,000 nucleic acid residues, at least 50,000 nucleic acid residues, at least 100,000 nucleic acid residues, at least 250,000 nucleic acid residues, or at least at least 500,000 nucleic acid residues.

In some embodiments, each bin in the plurality of bins has a corresponding buffer region. In such embodiments, each respective buffer region comprises at least 10 nucleic acid residues, at least 50 nucleic acid residues, at least 100 nucleic acid residues, at least 150 nucleic acid residues, at least 200 nucleic acid residues, at least 250 nucleic acid residues, at least 500 nucleic acid residues, or at least 1000 nucleic acid residues.

In some embodiments, the plurality of features are applied to the reference model, and the method further comprises determining the plurality of features from the plurality of copy number values by applying a dimensionality reduction method to the plurality of bin values thereby identifying all or a subset of the plurality of features in the form of a plurality of dimension reduction components.

In some embodiments, the method further comprises deriving the plurality of allele frequencies by using the second plurality of sequence reads to identify support for an allele for a variant in a variant set, thereby determining an observed frequency of the allele for the variant in the variant set. In such embodiments, each observed frequency corresponds to a respective allele frequency in the plurality of allele frequencies. In some such embodiments, a respective sequence read in the second plurality of sequence reads is deemed to support an allele of a first variant in the variant set when the respective sequence read contains the allele of the first variant, a respective sequence read in the second plurality of sequence reads is deemed not to support the allele of the first variant in the variant set when the respective sequence read maps on to the genomic region encompassing the allele but does not contain the allele of the first variant. Further, the observed frequency of the allele of the first variant is determined by a ratio or proportion between (i) a first number of unique cell-free nucleic acids, represented by the second plurality of sequence reads, that support the allele of the first variant and (ii) a second number of cell-free nucleic acids, represented by the second plurality of sequence reads, that map to the genomic region encompassing the allele irrespective of whether they support or do not support the allele of the first variant in the variant set, where the second number of cell-free nucleic acids includes the first number of cell-free nucleic acids.

In some embodiments each respective variant in the variant set corresponds to a particular region in the reference genome of the subject. In some embodiments, the variant set comprises at least one variant, at least 10 variants, at least 20 variants, at least 30 variants, at least 40 variants, at least 50 variants, at least 60 variants, at least 70 variants, at least 80 variants, at least 90 variants, at least 100 variants, at least 200 variants, at least 300 variants, at least 400 variants, at least 500 variants, at least 600 variants, at least 700 variants, at least 800 variants, at least 900 variants, at least 1000 variants, at least 200 variants, at least 3000 variants, at least 400 variants, at least 5000 variants, at least 6000 variants, at least 7000 variants, at least 8000 variants, at least 9000 variants, at least 10,000 variants, at least 20,000 variants, at least 30,000 variants, at least 40,000 variants, at least 50,000 variants, at least 60,000 variants, at least 70,000 variants, at least 80,000 variants, at least 90,000 variants, or at least 100,000 variants.

In some embodiments, the deriving the plurality of bin values further comprises using the first plurality of sequence reads to determine a respective number of cell-free nucleic acids represented by the first plurality of sequence reads that map to each respective bin in the plurality of bins, thereby determining a corresponding bin count for each respective bin. Further, each respective bin count is normalized to obtain the plurality of bin values. Further still in such embodiments, the deriving the plurality of allele frequencies further comprises using the second plurality of sequence reads to identify support for an allele for a variant in a variant set, thereby determining an observed frequency of the allele for the variant in the variant set, where each observed frequency corresponds to a respective allele frequency in the plurality of allele frequencies.

In some embodiments, the first plurality of sequence reads provides an average coverage of between 20× and 70,000× across the plurality of bins and the second plurality of sequence reads provides an average coverage of between 30,000× and 70,000× across the plurality of bins.

In some embodiments, the first plurality of sequence reads provides an average coverage of between 20× and 70,000× across the plurality of bins and the second plurality of sequence reads provides an average coverage of between 30,000× and 70,000× across the plurality of alleles.

In some embodiments, the corresponding region of the reference genome, or a portion thereof, for each respective bin in the plurality of bins is complementary or substantially complementary to the sequences of two or more probes in a plurality of probes used in the first nucleic acid sequencing to generate the plurality of bin values.

In some embodiments that make use of probes for targeted sequencing, the respective corresponding region of the reference genome, or a portion thereof, for each corresponding bin in a first set of bins in the plurality of bins is complementary or substantially complementary to the sequences of two or more probes in the plurality of probes used in the targeted panel sequencing to generate the plurality of bin values. Further, the respective corresponding region of the reference genome, or a portion thereof, for each corresponding bin in a second set of bins in the plurality of bins is not represented by a sequence of any probe in the plurality of probes.

In some embodiments, the tumor fraction of the subject is between 0.001 and 1.0.

In some embodiments, the first biological sample and the second biological sample comprise one or a combination selected from the group consisting of blood, whole blood, plasma, serum, urine, cerebrospinal fluid, fecal, saliva, tears, pleural fluid, pericardial fluid, and peritoneal fluid of the subject.

In some embodiments, the determining the tumor fraction of the subject further identifies a cancer of origin of the subject. In some such embodiments, the cancer of origin consists of a first cancer condition selected from the group comprising non-cancer, breast cancer, lung cancer, prostate cancer, colorectal cancer, renal cancer, uterine cancer, pancreatic cancer, cancer of the esophagus, lymphoma, head/neck cancer, ovarian cancer, hepatobiliary cancer, melanoma, cervical cancer, multiple myeloma, leukemia, thyroid cancer, bladder cancer, gastric cancer, nasopharyngeal cancer, liver cancer, or a combination thereof. In alternative embodiments, the cancer of origin comprises at least a first cancer condition and a second cancer condition each selected from the group comprising breast cancer, lung cancer, prostate cancer, colorectal cancer, renal cancer, uterine cancer, pancreatic cancer, cancer of the esophagus, lymphoma, head/neck cancer, ovarian cancer, hepatobiliary cancer, melanoma, cervical cancer, multiple myeloma, leukemia, thyroid cancer, bladder cancer, gastric cancer, nasopharyngeal cancer, liver cancer, or a combination thereof.

In some embodiments that make use of probes for targeted sequencing, each respective probe in the plurality of probes includes a respective nucleic acid sequence that is complementary or substantially complementary to the respective genomic region.

In some embodiments that make use of probes for targeted sequencing, a respective probe in the plurality of probes includes a corresponding nucleic acid sequence that is complementary or substantially complementary to the reference genome, or a portion thereof, as represented by a bin in the plurality of bins with the exception of one or more transitions, and each respective transition in the one or more transitions occurs at a respective un-methylated CpG dinucleotide site in the reference genome.

In some embodiments that make use of probes for targeted sequencing, a respective probe in the plurality of probes includes a corresponding nucleic acid sequence that is complementary or substantially complementary to the reference genome, or a portion thereof, as represented by a bin in the plurality of bins with the exception of one or more transitions and each respective transition in the one or more transitions occurs at a respective methylated CpG dinucleotide site in the reference genome.

In some embodiments that make use of probes for targeted sequencing, each probe in the plurality of probes includes a respective nucleic acid sequence that is complementary or substantially complementary to the reference genome, or a portion thereof, as represented by a bin in the plurality of bins, with the exception that the probe includes an adenine to complement a thymine corresponding to a methylated or unmethylated cytosine in a selected cell-free nucleic acid.

In some embodiments, each respective bin in the plurality of bins represents a non-overlapping corresponding region of the reference genome of the species.

In some embodiments, each respective bin value in the first plurality of bin values is a count of a number of cell-free-nucleic acids represented by the first plurality of sequence reads that map to a corresponding bin in the plurality of bins.

In some embodiments, the first nucleic acid sequencing is methylation sequencing, and each respective bin value in the first plurality of bin values is a count of a number of cell-free-nucleic acids represented by the first plurality of sequence reads that map to a corresponding bin in the plurality of bins after application of one or more filter conditions.

In some embodiments, the methylation sequencing produces a corresponding methylation pattern for each respective cell-free nucleic acid in the first plurality of cell-free nucleic acids, and a filter condition in the one or more filter conditions is application of a p-value threshold to the corresponding methylation pattern, where the p-value threshold is representative of how frequently a methylation pattern is observed in a cohort of non-cancer subjects. In some such embodiments, the p-value threshold is between 0.001 and 0.20. In some such embodiments, the p-value threshold is below 0.01.

In some embodiments, the methylation sequencing produces a corresponding methylation pattern for each respective cell-free nucleic acid in the first plurality of cell-free nucleic acids, and a filter condition in the one or more filter conditions is application of a requirement that the respective cell-free nucleic acid is represented by a threshold number of sequence reads in the corresponding first plurality of sequence reads. In some such embodiments, the threshold number is 2, 3, 4, 5, 6, 7, 8, 9, 10, or an integer between 10 and 100.

In some embodiments, the methylation sequencing produces a corresponding methylation pattern for each respective cell-free nucleic acid in the first plurality of cell-free nucleic acids, and a filter condition in the one or more filter conditions is application of a requirement that the respective cell-free nucleic acid is represented by a threshold number of cell-free nucleic acids in the first plurality of sequence reads. In some such embodiments, the threshold number is 2, 3, 4, 5, 6, 7, 8, 9, 10, or an integer between 10 and 100.

In some embodiments, the methylation sequencing produces a corresponding methylation pattern for each respective cell-free nucleic acid in the first plurality of cell-free nucleic acids, and a filter condition in the one or more filter conditions is application of a requirement that the respective cell-free nucleic acid have a threshold number of CpG sites. In some such embodiments, the threshold number of CpG sites is at least 1, 2, 3, 4, 5, 6, 7, 8, 9 or 10 CpG sites.

In some embodiments, the methylation sequencing produces a corresponding methylation pattern for each respective cell-free nucleic acid in the first plurality of cell-free nucleic acids, and a filter condition in the one or more filter conditions is a requirement that the respective cell-free nucleic acid have a length of less than a threshold number of base pairs. In some such embodiments the threshold number of base pairs is one thousand, two thousand, three thousand, or four thousand contiguous base pairs in length.

In some embodiments, each respective bin value in the first plurality of bin values is a count of a number of cell-free-nucleic acids represented by the first plurality of sequence reads that both (i) map to a corresponding bin in the plurality of bins and (ii) have a methylation pattern satisfying a p-value threshold that is representative of how frequently a methylation pattern is observed in a cohort of non-cancer subjects. In some such embodiments, the p-value threshold is below 0.01. In some such embodiments, the cohort comprises at least twenty subjects and the population of methylation patterns comprises more than 10,000 different methylation sequences. In some such embodiments, the p-value threshold is satisfied for a methylation pattern from the subject when the methylation pattern from the subject has a p-value of 0.10 or less, 0.05 or less, or 0.01 or less.

In some embodiments, the reference model is a multivariate logistic regression, a neural network, a convolutional neural network, a support vector machine, a decision tree, a regression algorithm, or a supervised clustering model.

In some embodiments, each allele in the plurality of alleles is a single nucleotide variant associated with a predetermined genomic location, an insertion mutation associated with a predetermined genomic location, a deletion mutation associated with a predetermined genomic location, a somatic copy number alteration, a nucleic acid rearrangement associated with a predetermined genomic locus, or an aberrant methylation pattern associated with a predetermined genomic location.

In some embodiments, the plurality of alleles comprises between 2 and 20,000 alleles, and each allele is for a different genetic variation in the genome of the species.

In some embodiments, the plurality of alleles consists of between 15 and 5,000 alleles, and each allele is for a different genetic variation in the genome of the species.

In some embodiments, the plurality of alleles consists of between 20 and 1,000 alleles, and each allele is for a different genetic variation in the genome of the species.

In some embodiments, the method determines that the tumor fraction is less than 1×10⁻³.

In some embodiments, the method further comprises repeating the above-described method at each respective time point in a plurality of time points across an epoch, thereby obtaining a corresponding tumor fraction, in a plurality of tumor fractions, for the subject at each respective time point. In some such embodiments, the method further comprises using the plurality of tumor fractions to determine a state or progression of a disease condition in the subject during the epoch in the form of an increase or decrease of the first tumor fraction over the epoch. In some such embodiments, the epoch is a period of months (e.g., less than four months) and each time point in the plurality of time points is a different time point in the period of months. In some such embodiments, the epoch is a period of years (e.g., between two and ten years, between two and twenty years, etc.) and each time point in the plurality of time points is a different time point in the period of years. In some embodiments, the epoch is a period of hours (e.g., between one hour and six hours, between 1 and 24 hours, etc.) and each time point in the plurality of time points is a different time point in the period of hours.

In some embodiments where tumor fraction is determined at a plurality of time points, the method further comprising changing a diagnosis of the subject when the first tumor fraction of the subject is observed to change by a threshold amount (e.g., greater than ten percent, greater than twenty percent, greater than thirty percent, greater than forty percent, greater than fifty percent, greater than two-fold, greater than three-fold, or greater than five-fold, etc.) across the epoch.

In some embodiments where tumor fraction is determined at a plurality of time points, the method further comprises changing a prognosis of the subject when the first tumor fraction of the subject is observed to change by a threshold amount (e.g., greater than ten percent, greater than twenty percent, greater than thirty percent, greater than forty percent, greater than fifty percent, greater than two-fold, greater than three-fold, or greater than five-fold, etc.) across the epoch.

In some embodiments where tumor fraction is determined at a plurality of time points, the method further comprises changing a treatment of the subject when the first tumor fraction of the subject is observed to change by a threshold amount (e.g., greater than ten percent, greater than twenty percent, greater than thirty percent, greater than forty percent, greater than fifty percent, greater than two-fold, greater than three-fold, or greater than five-fold, etc.) across the epoch.

In some embodiments, the tumor fraction is between 0.003 and 1.0.

Another aspect of the present disclosure provides a non-transitory computer readable storage medium storing at least one program for determining a tumor fraction for a subject of a species. The at least one program is configured for execution by a computer. The at least one program comprises instructions for obtaining, in electronic form, a first dataset that comprises a plurality of bin values. Each respective bin value in the plurality of bin values being for a corresponding bin in a plurality of bins. Each respective bin in the plurality of bins represents a corresponding region of a reference genome of the species. The plurality of bin values is derived from alignment of a first plurality of sequence reads, determined by a first nucleic acid sequencing of a first plurality of cell-free nucleic acids in a first biological sample, to a reference genome of the species. The first biological sample comprises a liquid sample of the subject and the first plurality of cell-free nucleic acids comprises at least 1000 cell-free nucleic acids.

The at least one program comprises instructions for determining a plurality of copy number values at least in part from the plurality of bins values.

The at least one program comprises instructions for obtaining, in electronic form, a second dataset that comprises a plurality of allele frequencies for a plurality of alleles, where the plurality of allele frequencies is derived from alignment of a second plurality of sequence reads, determined by a second nucleic acid sequencing of a second plurality of cell-free nucleic acids in a second biological sample, to the reference genome, where the second biological sample comprises a liquid sample of the subject and the second plurality of cell-free nucleic acids comprises at least 1000 cell-free nucleic acids. The at least one program comprises instructions for applying, to a reference model, at least the plurality of copy number values and the plurality of allele frequencies, or a plurality of features derived therefrom, thereby determining the tumor fraction of the subject.

Another aspect of the present disclosure provides a computing system, comprising at least one processor and memory storing at least program to be executed by the at least one processor. The at least one program comprises instructions for determining a tumor fraction for a subject of a species by a method. In the method there is obtained, in electronic form, a first dataset that comprises a plurality of bin values. Each respective bin value in the plurality of bin values is for a corresponding bin in a plurality of bins. Each respective bin in the plurality of bins represents a corresponding region of a reference genome of the species. The plurality of bin values is derived from alignment of a first plurality of sequence reads, determined by a first nucleic acid sequencing of a first plurality of cell-free nucleic acids in a first biological sample, to a reference genome of the species. The first biological sample comprises a liquid sample of the subject and the first plurality of cell-free nucleic acids comprises at least 1000 cell-free nucleic acids. Further in the method, a plurality of copy number values is determined at least in part from the plurality of bins values. Further in the method there is obtained, in electronic form, a second dataset that comprises a plurality of allele frequencies for a plurality of alleles. The plurality of allele frequencies is derived from alignment of a second plurality of sequence reads, determined by a second nucleic acid sequencing of a second plurality of cell-free nucleic acids in a second biological sample, to the reference genome. The second biological sample comprises a liquid sample of the subject and the second plurality of cell-free nucleic acids comprises at least 1000 cell-free nucleic acids. Further in the method there is applied, to a reference model, at least the plurality of copy number values and the plurality of allele frequencies, or a plurality of features derived therefrom, thereby determining the tumor fraction of the subject.

### B. Embodiments for Training a Reference Model

Another aspect of the present disclosure provides a method of training a reference model to determine a tumor fraction of a test subject. The method is performed at a computer system comprising at least one processor and a memory storing at least one program for execution by the at least one processor. The at least one program comprising instructions for performing the method. In the method, a training dataset is obtained in electronic form. The training dataset comprises, for each respective reference subject in a plurality of reference subjects, (i) a corresponding plurality of bin values, each respective bin value in the corresponding plurality of bin values being for a corresponding bin in a plurality of bins, (ii) a corresponding plurality of allele frequencies for a corresponding plurality of alleles, and (iii) a corresponding tumor fraction value for the respective reference subject. Each respective bin in the plurality of bins represents a corresponding region of a reference genome of the species. Each corresponding plurality of bin values is derived from alignment of a corresponding first plurality of sequence reads, determined by a corresponding first nucleic acid sequencing of a corresponding first plurality of cell-free nucleic acids in a corresponding first biological sample, to a reference genome of the species. The first biological sample comprises a liquid sample of a respective reference subject in the plurality of reference subjects and the corresponding first plurality of cell-free nucleic acids comprises at least 1000 cell-free nucleic acids.

Each corresponding plurality of allele frequencies is derived from alignment of a corresponding second plurality of sequence reads, determined by a corresponding second nucleic acid sequencing of a corresponding second plurality of cell-free nucleic acids in a second biological sample, to the reference genome. The corresponding second biological sample comprises a liquid sample of a respective reference subject in the plurality of reference subjects and the corresponding second plurality of cell-free nucleic acids comprises at least 1000 cell-free nucleic acids.

In the method, there is determined, for each respective reference subject in the plurality of reference subjects, a respective plurality of copy number values at least in part from the corresponding plurality of bins values for the respective reference subject.

In the method, a reference model is obtained using at least (i) the respective plurality of copy number values, (ii) the respective plurality of allele frequencies, or a respective plurality of features derived from (i) and (ii), and (iii) the tumor fraction value of each respective reference subject in the plurality of reference subjects.

In some embodiments, each corresponding first nucleic acid sequencing and second nucleic acid sequencing is a targeted panel sequencing that provides both the plurality of bin values and the plurality of allele frequencies. Further, the targeted panel sequencing uses a plurality of probes, each probe in the plurality of probes includes a nucleic acid sequence that corresponds to the sequence, or a complementary sequence thereof, of a portion of the reference genome represented by a corresponding one or more bins in the plurality of bins. In some such embodiments, the respective corresponding region of the reference genome, or a portion thereof, of each corresponding bin in a first set of bins in the plurality of bins is complementary or substantially complementary to the sequences of two or more probes in the plurality of probes and the respective corresponding region of the reference genome, or a portion thereof, for each corresponding bin in a second set of bins in the plurality of bins is not represented by a sequence of any in the plurality of probes.

In some embodiments, the reference model comprises a multivariate logistic regression, a neural network, a convolutional neural network, a support vector machine, a decision tree, a regression algorithm, or a supervised clustering model.

In some embodiments, the corresponding first biological sample of each respective reference subject comprises a liquid sample of the respective reference subject.

In some embodiments, the corresponding plurality of bin values for each respective reference subject is derived by using the corresponding first plurality of sequence reads to determine a respective number of cell-free nucleic acids represented by the corresponding first plurality of sequence reads that map to each respective bin in the plurality of bins, thereby determining each respective bin value in the plurality of bin values.

In some embodiments, the plurality of allele frequencies for each respective reference subject are derived by using the corresponding second plurality of sequence reads to identify support for an allele for a variant in a variant set, thereby determining an observed frequency of the allele for the variant in the variant set, where each observed frequency corresponds to a respective allele frequency in the plurality of allele frequencies. In some such embodiments, a respective sequence read in the corresponding second plurality of sequence reads is deemed to support an allele of a first variant in the variant set when the respective sequence read contains the allele of the first variant. Further, a respective sequence read in the corresponding second plurality of sequence reads is deemed not to support the allele of the first variant in the variant set when the respective sequence read maps on to the genomic region encompassing the allele but does not contain the allele of the first variant. In such embodiments, the observed frequency of the allele of the first variant is determined by a ratio or proportion between (i) a corresponding first number of unique cell-free nucleic acids, represented by the corresponding second plurality of sequence reads, that support the allele of the first variant and (ii) a corresponding second number of unique cell-free nucleic acids, represented by the corresponding second plurality of sequence reads, that map to the genomic region encompassing the allele irrespective of whether they support or do not support the allele, where the corresponding second number of unique cell-free nucleic acids includes the corresponding first number of cell-free nucleic acids.

In some embodiments, each respective variant in the variant set corresponds to a particular region in the reference genome of the plurality of reference subjects. In some embodiments, the variant set comprises at least one variant, at least 10 variants, at least 20 variants, at least 30 variants, at least 40 variants, at least 50 variants, at least 60 variants, at least 70 variants, at least 80 variants, at least 90 variants, at least 100 variants, at least 200 variants, at least 300 variants, at least 400 variants, at least 500 variants, at least 600 variants, at least 700 variants, at least 800 variants, at least 900 variants, at least 1000 variants, at least 200 variants, at least 3000 variants, at least 400 variants, at least 5000 variants, at least 6000 variants, at least 7000 variants, at least 8000 variants, at least 9000 variants, at least 10,000 variants, at least 20,000 variants, at least 30,000 variants, at least 40,000 variants, at least 50,000 variants, at least 60,000 variants, at least 70,000 variants, at least 80,000 variants, at least 90,000 variants, or at least 100,000 variants.

In some embodiments, for each respective reference subject in the plurality of reference subjects, the method comprises applying a dimensionality reduction method to the corresponding plurality of bin values, thereby identifying all or a subset of the corresponding plurality of features in the form of a corresponding plurality of dimension reduction components.

In some embodiments, the tumor fraction of each respective reference subject in the plurality of reference subjects is between 0.001 and 1.0.

In some embodiments, the corresponding first nucleic acid sequencing is a corresponding methylation sequencing. In some such embodiments, each respective bin value in the corresponding first plurality of bin values is a count of a number of cell-free-nucleic acids represented by the corresponding first plurality of sequence reads that map to a corresponding bin in the plurality of bins after application of one or more filter conditions.

In some embodiments, the corresponding methylation sequencing produces a corresponding methylation pattern for each respective cell-free nucleic acid in the corresponding first plurality of cell-free nucleic acids, and a filter condition in the one or more filter conditions is application of a p-value threshold to the corresponding methylation pattern. In such embodiments, the p-value threshold is representative of how frequently a methylation pattern is observed in a cohort of non-cancer subjects. In some such embodiments, the p-value threshold is below 0.01.

In some embodiments, the corresponding methylation sequencing produces a corresponding methylation pattern for each respective cell-free nucleic acid in the corresponding first plurality of cell-free nucleic acids, and a filter condition in the one or more filter conditions is application of a requirement that the respective cell-free nucleic acid is represented by a threshold number of sequence reads in the corresponding first plurality of sequence reads. In some such embodiments, the threshold number is 2, 3, 4, 5, 6, 7, 8, 9, 10, or an integer between 10 and 100.

In some embodiments, the corresponding methylation sequencing produces a corresponding methylation pattern for each respective cell-free nucleic acid in the corresponding first plurality of cell-free nucleic acids, and a filter condition in the one or more filter conditions is application of a requirement that the respective cell-free nucleic acid is represented by a threshold number of cell-free nucleic acids in the corresponding first plurality of sequence reads. In some such embodiments, the threshold number is 2, 3, 4, 5, 6, 7, 8, 9, 10, or an integer between 10 and 100.

In some embodiments, the corresponding methylation sequencing produces a corresponding methylation pattern for each respective cell-free nucleic acid in the first plurality of cell-free nucleic acids, and a filter condition in the one or more filter conditions is application of a requirement that the respective cell-free nucleic acid have a threshold number of CpG sites. In some such embodiments, the threshold number of CpG sites is at least 1, 2, 3, 4, 5, 6, 7, 8, 9 or 10 CpG sites.

In some embodiments, the corresponding methylation sequencing produces a corresponding methylation pattern for each respective cell-free nucleic acid in the corresponding first plurality of cell-free nucleic acids, and a filter condition in the one or more filter conditions is a requirement that the respective cell-free nucleic acid have a length of less than a threshold number of base pairs. In some such embodiments, the threshold number of base pairs is 1 thousand, 2 thousand, 3 thousand, or 4 thousand contiguous base pairs in length.

In some embodiments, each respective bin value in the corresponding first plurality of bin values is a count of a number of cell-free-nucleic acids represented by the corresponding first plurality of sequence reads that both (i) map to a corresponding bin in the plurality of bins and (ii) have a methylation pattern satisfying a p-value threshold that is representative of how frequently a methylation pattern is observed in a cohort of non-cancer subjects. In some such embodiments, the p-value threshold is between 0.001 and 0.20. In some such embodiments, the p-value threshold is below 0.01. In some such embodiments, the cohort comprises at least twenty subjects and the population of methylation patterns comprises more than 10,000 different methylation sequences. In some such embodiments, the p-value threshold is satisfied for a methylation pattern from the respective training subject when the methylation pattern from the respective training subject has a p-value of 0.10 or less, 0.05 or less, or 0.01 or less.

Another aspect of the present disclosure provides a computing system comprising at least one processor and memory storing at least one program to be executed by the at least one processor, the at least one program comprising instructions for determining a tumor fraction for a subject of a species by any of the methods disclosed above.

Still another aspect of the present disclosure provides a non-transitory computer readable storage medium storing at least one program for determining a tumor fraction for a subject of a species. The at least one programs is configured for execution by a computer. The at least one program comprises instructions for performing any of the methods disclosed above.

As disclosed herein, any embodiment disclosed herein when applicable can be applied to any aspect.

Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference. In the event of a conflict between a term herein and a term in an incorporated reference, the term herein controls.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIG. 1 is a block diagram illustrating an example of a computing system in accordance with some embodiments of the present disclosure.

FIGS. 2A, 2B, 2C, and 2D collectively illustrate examples of flowcharts of methods of determining a tumor fraction of a subject, in accordance with some embodiments of the present disclosure.

FIG. 3 illustrates an example of tumor fraction being correlated with allele frequency (in particular the second highest allele frequency for each subject), in accordance with some embodiments of the present disclosure. Shown in FIG. 3 are samples where the estimated tumor fraction is determined, for each patient, from a tissue sample of the respective patient. The samples are further identified by known cancer stage, thus indicating that there is a correlation between allele frequency and tumor fraction regardless of the patient's cancer stage.

FIG. 4 illustrates an example of tumor fraction being correlated with both the first and second highest allele frequencies (as calculated across the population of subjects), in accordance with some embodiments of the present disclosure. In FIG. 4, each known tumor fraction is determined from a tissue sample.

FIG. 5 illustrates an example of tumor fraction correlated with copy number instability, in accordance with some embodiments of the present disclosure. In FIG. 5, each tumor fraction is determined from a tissue sample. As with the examples shown in FIG. 4, this correlation holds primarily for subjects with a tumor fraction above 0.01.

FIGS. 6A and 6B illustrate, in accordance with some embodiments of the present disclosure, an example of tumor fraction determined based on allele frequency analysis being correlated with tumor fraction derived from tissue analysis (e.g., as shown in FIG. 4) for the specific case of lung cancer. FIG. 6A includes samples from all stages of lung cancer. FIG. 6B includes samples from just stages III and IV of lung cancer.

FIGS. 7A, 7B, and 7C illustrate that, in accordance with some embodiments of the present disclosure, a combination of allele frequency and copy number instability correlates, for each patient, with tumor fraction estimated from tissue samples. As demonstrated above in FIGS. 4 and 5, respectively, allele frequency and copy number instability are often well correlated with tumor fraction. However, there are instances where allele frequency is not perfectly known for a particular patient, or where allele frequency alone does not suffice to determine tumor fraction with sufficient accuracy. Similarly, copy number instability alone is not always correlated tightly with tumor fraction. FIG. 7A illustrates the correlation of top 20 allele frequencies per patient with tumor fraction. FIG. 7B illustrates the correlation of copy number instability calculated for each subject with tumor fraction. FIG. 7C illustrates that the combination of these metrics results in an improved correlation with tumor fraction.

FIGS. 8A, 8B, and 8C illustrate that, in accordance with some embodiments of the present disclosure, a combination of allele frequency and copy number instability correlates, for each patient, with tumor fraction estimated from tissue samples. FIG. 8A illustrates the correlation of the top allele frequencies for each gene of each patient with tumor fraction.

FIG. 8B illustrates the correlation of copy number instability calculated for each subject with tumor fraction. FIG. 8C illustrates that the combination of these metrics results in an improved correlation with tumor fraction.

FIG. 9 illustrates that, in accordance with some embodiments of the present disclosure, allele frequency can be predicted using methylation data from whole genome methylation sequencing, thus indicating that methylation data can be used to predict tumor fraction, either alone or in combination with copy number instability or allele frequency.

FIG. 10 illustrates GC normalization of bin counts, as part of determining normalized bin values for use in accordance with some embodiments of the present disclosure.

FIG. 11 is a flowchart describing a process of sequencing nucleic acids, in accordance with an aspect of the present disclosure.

FIG. 12 is an illustration of a part of the process of sequencing nucleic acids to obtain methylation information and methylation state vectors, in accordance with an aspect of the present disclosure.

FIG. 13 is an illustration of bins (blocks) of a reference genome, in accordance with an aspect of the present disclosure.

### DETAILED DESCRIPTION

The gold standard for determining cell-free tumor fraction in cancer patients is a determination based on nucleic acid sequencing data of tumor tissue isolated from a biopsy sample (e.g., compared with nucleic acid sequencing data of nucleic acid fragments isolated from a blood sample). See e.g., Vaidyanathan et al. 2019 Lab Chip 19, 11-34; and Takahashi et al. 2013 PLoS One. 8(12): e82302. However, this method is insufficient for many patients. First, it is not always possible or convenient to obtain a biopsy (e.g., in particular for hematological tumors or for obtaining real-time data to observe patient response to treatment). Second, information for estimating tumor fraction is not always present in tissue samples (e.g., some cancers lack variants within the analyzed regions). Hence, other methods of determining tumor fraction are needed (see Example 1 where allele frequency and copy number are combined to provide a method of estimating tumor fraction from cell-free nucleic acid sequencing information).

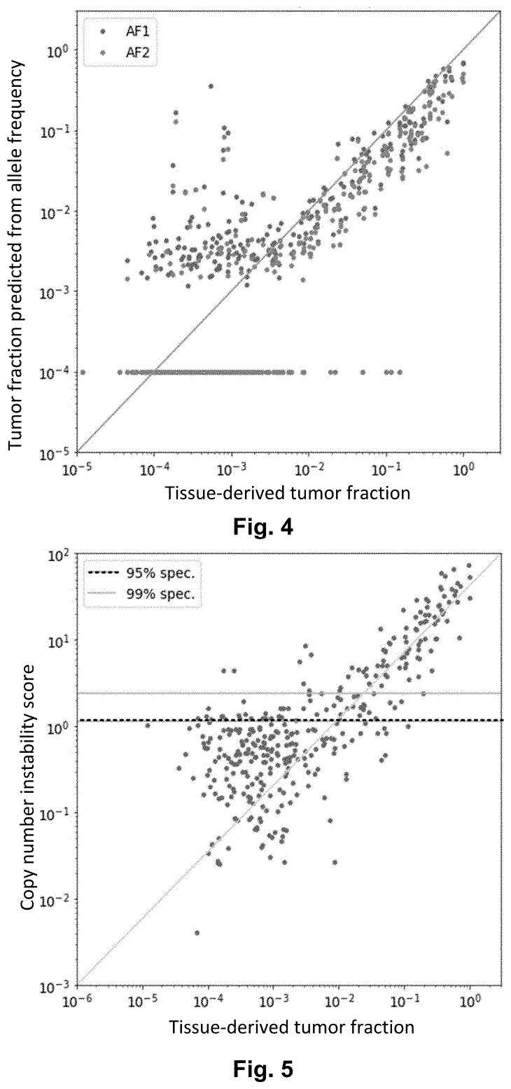

As described in the present disclosure, using information about both copy number and allele frequency enables improved estimates of tumor fraction for subjects. Each type of data contributes to the tumor fraction determination. Alone each data type can be used to determine tumor fraction (see FIGS. 4 and 5); however, when used in combination the accuracy of such a determination is improved (see e.g., Example 1). Given the importance of tumor fraction in predicting patient morbidity and in informing treatment options, any improvement in tumor fraction determination accuracy can have a positive impact on patient outcomes.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

The implementations described herein provide various technical solutions for training a reference model to determining a tumor fraction for a subject.

### Definitions

As used herein, the term “abnormal methylation pattern” or “anomalous methylation pattern” refers to a methylation state vector, methylation pattern, or a methylation status of a DNA molecule having the methylation state vector that is expected to be found in a sample less frequently than a threshold value. In a particular embodiment provided herein, the expectedness of finding a specific methylation state vector in a healthy control group comprising healthy individuals is represented by a p-value. In some embodiments, p-values of methylation state vectors are determined as described in Example 5 of PCT/US2020/034317, entitled “Systems and methods for Determining Whether a Subject has a Cancer Condition Using Transfer Learning,” filed on May 22, 2020, and which is incorporated by reference herein in its entirety. A low p-value score, thereby, generally corresponds to a methylation state vector that is relatively unexpected in comparison to other methylation state vectors within samples from healthy individuals in the healthy control group. A high p-value score generally corresponds to a methylation state vector that is relatively more expected in comparison to other methylation state vectors found in samples from healthy individuals in the healthy control group. A methylation state vector having a p-value lower than a threshold value (e.g., 0.1, 0.01, 0.001, 0.0001, etc.) can be defined as an abnormal methylation pattern. Various methods known in the art can be used to calculate a p-value or expectedness of a methylation pattern or a methylation state vector. Exemplary methods provided herein involve use of a Markov chain probability that assumes methylation statuses of CpG sites to be dependent on methylation statuses of neighboring CpG sites. Alternate methods provided herein calculate the expectedness of observing a specific methylation state vector in healthy individuals by utilizing a mixture-model including multiple mixture components, each being an independent-sites model where methylation at each CpG site is assumed to be independent of methylation statuses at other CpG sites. Methods provided herein use genomic regions having an anomalous methylation pattern. A genomic region can be determined to have an anomalous methylation pattern when cfDNA fragments corresponding to or originated from the genomic region have methylation state vectors that appear less frequently than a threshold value in reference samples. The reference samples can be samples from control subjects or healthy subjects. The frequency for a methylation state vector to appear in the reference samples can be represented as a p-value score. When cfDNA fragments corresponding to or originated from the genomic region do not have a single, uniform methylation state vector, the genomic region can have multiple p-value scores for multiple methylation state vectors. In this case, the multiple p-value scores can be summed or averaged before being compared to the threshold value. Various methods known in the art can be adopted to compare p-value scores corresponding to the genomic region and the threshold value, including but not limited to arithmetic mean, geometric mean, harmonic mean, median, mode, etc.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. The term “about” or “approximately” can mean within an order of magnitude, within 5-fold, or within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to 5%.

As used herein, the term “assay” refers to a technique for determining a property of a substance, e.g., a nucleic acid, a protein, a cell, a tissue, or an organ. An assay (e.g., a first assay or a second assay) can comprise a technique for determining the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art can be used to detect any of the properties of nucleic acids mentioned herein. Properties of a nucleic acids include, but are not limited to, a sequence, genomic identity, copy number, methylation state at one or more nucleotide positions, size of the nucleic acid, presence or absence of a mutation in the nucleic acid at one or more nucleotide positions, and pattern of fragmentation of a nucleic acid (e.g., the nucleotide position(s) at which a nucleic acid forms fragments). An assay or method can have a particular sensitivity and/or specificity, and their relative usefulness as a diagnostic tool can be measured using ROC-AUC statistics.

As used herein, the term “biological sample,” “patient sample,” or “sample” refers to any sample taken from a subject, which can reflect a biological state associated with the subject, and that includes cell-free DNA. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. In some embodiments, the biological sample consists of blood, whole blood, plasma, serum, urine, cerebrospinal fluid, fecal, saliva, sweat, tears, pleural fluid, pericardial fluid, or peritoneal fluid of the subject. In such embodiments, the biological sample is limited to blood, whole blood, plasma, serum, urine, cerebrospinal fluid, fecal, saliva, sweat, tears, pleural fluid, pericardial fluid, or peritoneal fluid of the subject and does not contain other components (e.g., solid tissues, etc.) of the subject. A biological sample can include any tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis. A biological sample can be obtained from a subject invasively (e.g., surgical means) or non-invasively (e.g., a blood draw, a swab, or collection of a discharged sample).

As used herein, the term “cancer” or “tumor” refers to an abnormal mass of tissue in which the growth of the mass surpasses and is not coordinated with the growth of normal tissue. A cancer or tumor can be defined as “benign” or “malignant” depending on the following characteristics: degree of cellular differentiation including morphology and functionality, rate of growth, local invasion, and metastasis. A “benign” tumor can be well differentiated, have characteristically slower growth than a malignant tumor and remain localized to the site of origin. In addition, in some cases a benign tumor does not have the capacity to infiltrate, invade, or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites.

As used herein, the terms “cell-free nucleic acid,” “cell-free DNA,” and “cfDNA” interchangeably refer to nucleic acid fragments that are found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject (e.g., bloodstream). Cell-free nucleic acids are interchangeably referred to herein as “circulating nucleic acids.” Examples of the cell-free nucleic acids include but are not limited to RNA, mitochondrial DNA, or genomic DNA. Cell-free nucleic acids can originate from one or more healthy cells and/or from one or more cancer cells.

As used herein, the term “classification” can refer to any number(s) or other characters(s) that are associated with a particular property of a sample. For example, a symbol (or the word “positive”) can signify that a sample is classified as having deletions or amplifications. In another example, the term “classification” can refer to an amount of tumor tissue in the subject and/or sample, a size of the tumor in the subject and/or sample, a stage of the tumor in the subject, a tumor load in the subject and/or sample, and presence of tumor metastasis in the subject. The classification can be binary (e.g., positive or negative) or have more levels of classification (e.g., a scale from 1 to 10 or 0 to 1). The terms “cutoff” and “threshold” can refer to predetermined numbers used in an operation. For example, a cutoff size can refer to a size above which fragments are excluded. A threshold value can be a value above or below which a particular classification applies. Either of these terms can be used in either of these contexts.

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map sequence reads obtained from sequencing a sample from the subject. A reference genome can refer to a haploid or diploid genome to which sequence reads from the biological sample and a constitutional sample can be aligned and compared. An example of constitutional sample can be DNA of white blood cells obtained from the subject. For a haploid genome, there can be only one nucleotide at each locus. For a diploid genome, heterozygous loci can be identified; each heterozygous locus can have two alleles, where either allele can allow a match for alignment to the locus.

As used herein, the term “CpG site” refers to a region of a DNA molecule where a cytosine nucleotide is followed by a guanine nucleotide in the linear sequence of bases along its 5′ to 3′ direction. “CpG” is a shorthand for 5′-C-phosphate-G-3′ that is cytosine and guanine separated by only one phosphate group; phosphate links any two nucleotides together in DNA. Cytosines in CpG dinucleotides can be methylated to form 5-methylcytosine.

As used herein, the term “false positive” (FP) refers to a subject that does not have a condition. False positive can refer to a subject that does not have a tumor, a cancer, a precancerous condition (e.g., a precancerous lesion), a localized or a metastasized cancer, a non-malignant disease, or is otherwise healthy. The term false positive can refer to a subject that does not have a condition, but is identified as having the condition by an assay or method of the present disclosure.

As used herein, the term “false negative” (FN) refers to a subject that has a condition. False negative can refer to a subject that has a tumor, a cancer, a precancerous condition (e.g., a precancerous lesion), a localized or a metastasized cancer, or a non-malignant disease. The term false negative can refer to a subject that has a condition, but is identified as not having the condition by an assay or method of the present disclosure.

As used herein, the term “fragment” is used interchangeably with “nucleic acid fragment” (e.g., a DNA fragment) and “cell-free nucleic acid molecule”, and refers to a portion of a polynucleotide or polypeptide sequence that comprises at least three consecutive nucleotides. In the context of sequencing of nucleic cell-free nucleic acid fragments found in a biological sample, the terms “fragment” and “nucleic acid fragment” interchangeably refer to a cell-free nucleic acid molecule that is found in the biological sample. In such a context, the sequencing (e.g., whole genome sequencing, targeted sequencing, etc.) forms one or more copies of all or a portion of such a nucleic acid fragment in the form of one or more corresponding sequence reads. Such sequence reads, which in fact may be PCR duplicates of the original nucleic acid fragment, therefore “represent” or “support” the nucleic acid fragment. There may be a plurality of sequence reads that each represent or support a particular nucleic acid fragment in the biological sample (e.g., PCR duplicates).

As used herein, the phrase “healthy,” refers to a subject possessing good health. A healthy subject can demonstrate an absence of any malignant or non-malignant disease. A “healthy individual” can have other diseases or conditions, unrelated to the condition being assayed, which can normally not be considered “healthy.”

As used herein, the term “hypomethylated” or “hypermethylated” refers to a methylation status of a DNA molecule containing multiple CpG sites (e.g., more than 3, 4, 5, 6, 7, 8, 9, 10, etc.) where a high percentage of the CpG sites (e.g., more than 80%, 85%, 90%, or 95%, or any other percentage within the range of 50%-100%) are unmethylated or methylated, respectively.

As used herein, the term “level of cancer” refers to whether cancer exists (e.g., presence or absence), a stage of a cancer, a size of tumor, presence or absence of metastasis, the total tumor burden of the body, and/or other measure of a severity of a cancer (e.g., recurrence of cancer). The level of cancer can be a number or other indicia, such as symbols, alphabet letters, and colors. The level can be zero. The level of cancer can also include premalignant or precancerous conditions (states) associated with mutations or a number of mutations. The level of cancer can be used in various ways. For example, screening can check if cancer is present in someone who is not known previously to have cancer. Assessment can investigate someone who has been diagnosed with cancer to monitor the progress of cancer over time, study the effectiveness of therapies or to determine the prognosis. In some embodiments, the prognosis can be expressed as the chance of a subject dying of cancer, or the chance of the cancer progressing after a specific duration or time, or the chance of cancer metastasizing. Detection can comprise ‘screening’ or can comprise checking if someone, with suggestive features of cancer (e.g., symptoms or other positive tests), has cancer. A “level of pathology” can refer to level of pathology associated with a pathogen, where the level can be as described above for cancer. When the cancer is associated with a pathogen, a level of cancer can be a type of a level of pathology.

As used herein, a “methylome” can be a measure of an amount or extent of DNA methylation at a plurality of sites or loci in a genome. The methylome can correspond to all or a part of a genome, a substantial part of a genome, or relatively small portion(s) of a genome. A “tumor methylome” can be a methylome of a tumor of a subject (e.g., a human). A tumor methylome can be determined using tumor tissue or cell-free tumor DNA in plasma. A tumor methylome can be one example of a methylome of interest. A methylome of interest can be a methylome of an organ that can contribute nucleic acid, e.g., DNA into a bodily fluid (e.g., a methylome of brain cells, a bone, lungs, heart, muscles, kidneys, etc.). The organ can be a transplanted organ.

As used herein, the term “methylation” refers to a modification of deoxyribonucleic acid (DNA) where a hydrogen atom on the pyrimidine ring of a cytosine base is converted to a methyl group, forming 5-methylcytosine. In particular, methylation tends to occur at dinucleotides of cytosine and guanine referred to herein as “CpG sites.” In other instances, methylation may occur at a cytosine not part of a CpG site or at another nucleotide that is not cytosine; however, these are rarer occurrences. In this present disclosure, methylation is discussed in reference to CpG sites for the sake of clarity. Anomalous cfDNA methylation can identified as hypermethylation or hypomethylation, both of which may be indicative of cancer status. As is well known in the art, DNA methylation anomalies (compared to healthy controls) can cause different effects, which may contribute to cancer.

As used herein the term “methylation index” for each genomic site (e.g., a CpG site, a region of DNA where a cytosine nucleotide is followed by a guanine nucleotide in the linear sequence of bases along its 5′ to 3′ direction) can refer to the proportion of sequence reads showing methylation at the site over the total number of reads covering that site. The “methylation density” of a region can be the number of reads at sites within a region showing methylation divided by the total number of reads covering the sites in the region. The sites can have specific characteristics, (e.g., the sites can be CpG sites). The “CpG methylation density” of a region can be the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of unconverted cytosines (which can correspond to methylated cytosine) at CpG sites as a proportion of all CpG sites covered by sequence reads mapped to the 100-kb region. In some embodiments, this analysis is performed for other bin sizes, e.g., 50-kb or 1-Mb, etc. In some embodiments, a region is an entire genome or a chromosome or part of a chromosome (e.g., a chromosomal arm). A methylation index of a CpG site can be the same as the methylation density for a region when the region only includes that CpG site. The “proportion of methylated cytosines” can refer the number of cytosine sites, “C's,” that are shown to be methylated (for example unconverted after bisulfite conversion) over the total number of analyzed cytosine residues, e.g., including cytosines outside of the CpG context, in the region. The methylation index, methylation density, and proportion of methylated cytosines are examples of “methylation levels.” One of skill in the art would understand that these parameters are devised to assess the extent or level of methylation in a particular sample and accordingly can be broadly defined so long as such definitions enable the assessment of an extent or a level of methylation in a sample. Additionally, such assessment can be performed for different genomic regions (e.g., from individual CpG sites, to nucleic acid fragments, to an entire gene and beyond); for example, a methylation index can sometimes simply refer to the number of methylated genes per sample. See Marzese et al. 2012 J Mol Diagnos 14(6), 613-622.

As used herein, the term “methylation profile” (also called methylation status) can include information related to DNA methylation for a region. Information related to DNA methylation can include a methylation index of a CpG site, a methylation density of CpG sites in a region, a distribution of CpG sites over a contiguous region, a pattern or level of methylation for each individual CpG site within a region that contains more than one CpG site, and non-CpG methylation. A methylation profile of a substantial part of the genome can be considered equivalent to the methylome. “DNA methylation” in mammalian genomes can refer to the addition of a methyl group to position 5 of the heterocyclic ring of cytosine (e.g., to produce 5-methylcytosine) among CpG dinucleotides. Methylation of cytosine can occur in cytosines in other sequence contexts (e.g., 5′-CHG-3′ and 5′-CHH-3′) where H is adenine, cytosine, or thymine. Cytosine methylation can also be in the form of 5-hydroxymethylcytosine. Methylation of DNA can include methylation of non-cytosine nucleotides, such as N6-methyladenine. For example, methylation data (e.g., density, distribution, pattern, or level of methylation) from different genomic regions can be converted to one or more vector set and analyzed by methods and systems disclosed herein.

As used herein, the term “methylation state vector” or “methylation status vector” refers to a vector comprising multiple elements, where each element indicates methylation status of a methylation site in a DNA molecule comprising multiple methylation sites, in the order they appear from 5′ to 3′ in the DNA molecule. For example, <Mx, Mx+J, Mx+2>, <Mx, Mx+1, Ux+2>, . . . , <Ux, Ux+1, Ux+2> can be methylation vectors for DNA molecules comprising three methylation sites, where M represents a methylation site that is in a methylated state and U represents a methylation site in an unmethylated state. U.S. Patent Application No. 62/948,129, entitled “Cancer Classification Using Patch Convolutional Neural Networks,” filed Dec. 13, 2019, which is hereby incorporated by reference in its entirety, further discloses methods of determining methylation state vectors. For example, for each sequence read in a plurality of sequence reads obtained from a biological sample of a subject, a respective location and respective methylation state is determined for each of one or more CpG cites based on alignment to a reference genome (e.g., the reference genome of the subject). A respective methylation state vector is determined for each fragment, where the respective methylation state vector is associated with a location of the fragment in the reference genome (e.g., as specified by the position of the first CpG site in each fragment, or another similar metric) and comprises a number of CpG sites in the fragment as well as the methylation state of each CpG site in the fragment whether methylated (e.g., denoted as M), unmethylated (e.g., denoted as U), or indeterminate (e.g., denoted as I). Observed states are states of methylated and unmethylated; whereas, an unobserved state is indeterminate.

As used herein, the term “mutation,” refers to a detectable change in the genetic material of one or more cells. In a particular example, one or more mutations can be found in, and can identify, cancer cells (e.g., driver and passenger mutations). A mutation can be transmitted from apparent cell to a daughter cell. A person having skill in the art will appreciate that a genetic mutation (e.g., a driver mutation) in a parent cell can induce additional, different mutations (e.g., passenger mutations) in a daughter cell. A mutation generally occurs in a nucleic acid. In a particular example, a mutation can be a detectable change in one or more deoxyribonucleic acids or fragments thereof. A mutation generally refers to nucleotides that is added, deleted, substituted for, inverted, or transposed to a new position in a nucleic acid. A mutation can be a spontaneous mutation or an experimentally induced mutation. The term “variant” refers to a region of the genome that differs between individuals of the same species (e.g., a region of the genome that comprises one or more mutations). A region of the genome corresponding to a variant may be mutated in multiple ways at a single location (e.g., a single nucleotide may be converted to an ‘A’ or to a ‘G’) or may be mutated at multiple locations. The term “allele” refers to one of two or more forms of a gene, where each form includes a mutation. An allele may correspond, for example, to a single nucleotide polymorphism (SNP), where a single base is mutated. Each allele is a variant of a gene. Each variant may comprises more than one allele. A mutation in the sequence (e.g., in one or more genes) of a particular tissue is an example of a “tissue-specific allele.” For example, a tumor can have a mutation that results in an allele at a locus that does not occur in normal cells. Another example of a “tissue-specific allele” is a fetal-specific allele that occurs in the fetal tissue, but not the maternal tissue.

Various challenges arise in the identification of anomalously methylated cfDNA fragments. First, determining a subject's cfDNA to be anomalously methylated only holds weight in comparison with a group of control subjects, such that if the control group is small in number, the determination loses confidence with the small control group. Additionally, among a group of control subjects' methylation status can vary which can be difficult to account for when determining a subject's cfDNA to be anomalously methylated. On another note, methylation of a cytosine at a CpG site causally influences methylation at a subsequent CpG site.

Those of skill in the art will appreciate that the principles described herein are equally applicable for the detection of methylation in a non-CpG context, including non-cytosine methylation. Further, methylation state vectors may contain elements that are generally vectors of sites where methylation has or has not occurred (even if those sites are not CpG sites specifically). With that substitution, the rest of the processes described herein are the same, and consequently the inventive concepts described herein are applicable to those other forms of methylation.

The term “normalize” as used herein means transforming a value or a set of values to a common frame of reference for comparison purposes. For example, when a diagnostic ctDNA level is “normalized” with a baseline ctDNA level, the diagnostic ctDNA level is compared to the baseline ctDNA level so that the amount by which the diagnostic ctDNA level differs from the baseline ctDNA level can be determined.

As used herein, the terms “nucleic acid” and “nucleic acid molecule” are used interchangeably. The terms refer to nucleic acids of any composition form, such as deoxyribonucleic acid (DNA, e.g., complementary DNA (cDNA), genomic DNA (gDNA) and the like), and/or DNA analogs (e.g., containing base analogs, sugar analogs and/or a non-native backbone and the like), DNA hybrids and polyamide nucleic acids (PNAs), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments, nucleic acids comprise nucleosomes, fragments, or parts of nucleosomes or nucleosome-like structures. Nucleic acids sometimes comprise protein (e.g., histones, DNA binding proteins, and the like). Nucleic acids analyzed by processes described herein sometimes are substantially isolated and are not substantially associated with protein or other molecules. Nucleic acids also include derivatives, variants and analogs of DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides include deoxyadenosine, deoxycytidine, deoxyguanosine, and deoxythymidine.

As used herein, the term “reference genome” refers to any particular known, sequenced, or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As disclosed herein, the term “regions of a reference genome,” “genomic region,” or “chromosomal region” refers to any portion of a reference genome, contiguous or non-contiguous. It can also be referred to, for example, as a bin, a partition, a genomic portion, a portion of a reference genome, a portion of a chromosome and the like. In some embodiments, a genomic section is based on a particular length of genomic sequence. In some embodiments, a method can include analysis of multiple mapped sequence reads to a plurality of genomic regions. Genomic regions can be approximately the same length or the genomic sections can be different lengths. In some embodiments, genomic regions are of about equal length. In some embodiments, genomic regions of different lengths are adjusted or weighted. In some embodiments, a genomic region is about 10 kilobases (kb) to about 500 kb, about 20 kb to about 400 kb, about 30 kb to about 300 kb, about 40 kb to about 200 kb, and sometimes about 50 kb to about 100 kb. In some embodiments, a genomic region is about 100 kb to about 200 kb. A genomic region is not limited to contiguous runs of sequence. Thus, genomic regions can be made up of contiguous and/or non-contiguous sequences. A genomic region is not limited to a single chromosome. In some embodiments, a genomic region includes all or part of one chromosome, or all or part of two or more chromosomes. In some embodiments, genomic regions may span one, two, or more entire chromosomes. In addition, the genomic regions may span joint or disjointed portions of multiple chromosomes.

As used herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). In some embodiments, sequence reads (e.g., single-end or paired-end reads) can be generated from one or both strands of a targeted nucleic acid fragment. The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median, or average length of about 1000 bp, 2000 bp, 5000 bp, 10,000 bp, or 50,000 bp or more. Nanopore sequencing, for example, can provide sequence reads, that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the terms “sequencing,” “sequence determination,” and the like as used herein refers generally to any and all biochemical processes that may be used to determine the order of biological macromolecules such as nucleic acids or proteins. For example, sequencing data can include all or a portion of the nucleotide bases in a nucleic acid molecule such as a DNA fragment.

As used herein the terms “sequencing depth,” “coverage,” and “coverage rate” are used interchangeably herein to refer refers to the number of times a locus is covered by a consensus sequence read corresponding to a unique nucleic acid target molecule (“nucleic acid fragment”) aligned to the locus; e.g., the sequencing depth is equal to the number of unique nucleic acid target fragments (excluding PCR sequencing duplicates) covering the locus. The locus can be as small as a nucleotide, or as large as a chromosome arm, or as large as an entire genome. Sequencing depth can be expressed as “YX”, e.g., 50×, 100×, etc., where “Y” refers to the number of times a locus is covered with a sequence corresponding to a nucleic acid target; e.g., the number of times independent sequence information is obtained covering the particular locus. In some embodiments, the sequencing depth corresponds to the number of genomes that have been sequenced. Sequencing depth can also be applied to multiple loci, or the whole genome, in which case Y can refer to the mean or average number of times a loci or a haploid genome, or a whole genome, respectively, is sequenced. When a mean depth is quoted, the actual depth for different loci included in the dataset can span over a range of values. Ultra-deep sequencing can refer to at least 100× in sequencing depth at a locus.

As used herein, the term “true positive” (TP) refers to a subject having a condition. True positive can refer to a subject that has a tumor, a cancer, a precancerous condition (e.g., a precancerous lesion), a localized or a metastasized cancer, or a non-malignant disease. True positive can refer to a subject having a condition, and is identified as having the condition by an assay or method of the present disclosure.

As used herein, the term “true negative” (TN) refers to a subject that does not have a condition or does not have a detectable condition. True negative can refer to a subject that does not have a disease or a detectable disease, such as a tumor, a cancer, a precancerous condition (e.g., a precancerous lesion), a localized or a metastasized cancer, a non-malignant disease, or a subject that is otherwise healthy. True negative can refer to a subject that does not have a condition or does not have a detectable condition, or is identified as not having the condition by an assay or method of the present disclosure.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the term “single nucleotide variant” or “SNV” refers to a substitution of one nucleotide at a position (e.g., site) of a nucleotide sequence, e.g., a sequence corresponding to a target nucleic acid molecule from an individual, to a nucleotide that is different from the nucleotide at the corresponding position in a reference genome. A substitution from a first nucleobase X to a second nucleobase Y may be denoted as “X>Y.” For example, a cytosine to thymine SNV may be denoted as “C>T.” In some embodiments, an SNV does not result in a change in amino acid expression (a synonymous variant). In some embodiments, an SNV results in a change in amino acid expression (a non-synonymous variant).

As used herein, the terms “size profile” and “size distribution” can relate to the sizes of DNA fragments in a biological sample. A size profile can be a histogram that provides a distribution of an amount of DNA fragments at a variety of sizes. Various statistical parameters (also referred to as size parameters or just parameter) can distinguish one size profile to another. One parameter can be the percentage of DNA fragment of a particular size or range of sizes relative to all DNA fragments or relative to DNA fragments of another size or range.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity can characterize the ability of a method to correctly identify one or more markers indicative of cancer.

As used herein, the term “subject” refers to any living or non-living organism, including but not limited to a human (e.g., a male human, female human, fetus, pregnant female, child, or the like), a non-human animal, a plant, a bacterium, a fungus, or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale, and shark. In some embodiments, a subject is a male or female of any stage (e.g., a man, a women or a child).

As used herein, the term “tissue” can correspond to a group of cells that group together as a functional unit. More than one type of cell can be found in a single tissue. Different types of tissue may consist of different types of cells (e.g., hepatocytes, alveolar cells or blood cells), but also can correspond to tissue from different organisms (mother versus fetus) or to healthy cells versus tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” can be used to refer to a tissue from which a cell-free nucleic acid originates. In one example, viral nucleic acid fragments can be derived from blood tissue. In another example, viral nucleic acid fragments can be derived from tumor tissue.

As used herein, the term “vector” is an enumerated list of elements, such as an array of elements, where each element has an assigned meaning. As such, the term “vector” as used in the present disclosure is interchangeable with the term “tensor.” As an example, if a vector comprises the bin counts for 10,000 bins, there exists a predetermined element in the vector for each one of the 10,000 bins. For ease of presentation, in some instances a vector may be described as being one-dimensional. However, the present disclosure is not so limited. A vector of any dimension may be used in the present disclosure provided that a description of what each element in the vector represents is defined (e.g., that element 1 represents bin count of bin 1 of a plurality of bins, etc.).

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

### Exemplary System Embodiments

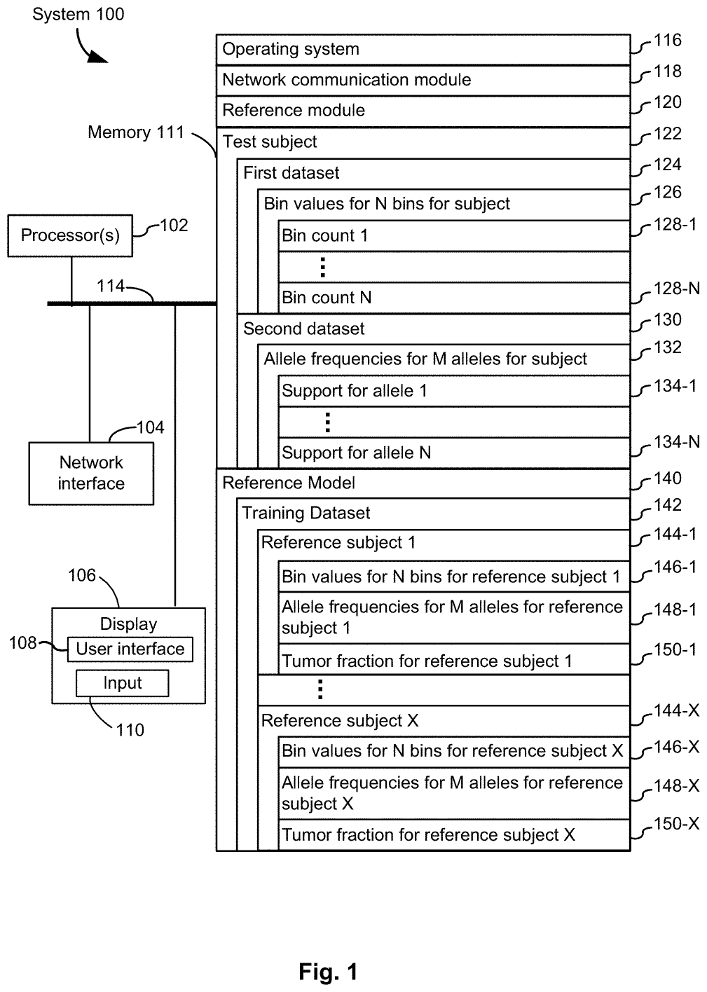

Details of an exemplary system are now described in conjunction with FIG. 1. FIG. 1 is a block diagram illustrating a system 100 in accordance with some implementations. The device 100 in some implementations includes at least one or more processing units CPU(s) 102 (also referred to as processors), one or more network interfaces 104, a display 106 having a user interface 108, an input device 110, a memory 111, and one or more communication buses 114 for interconnecting these components. The one or more communication buses 114 optionally include circuitry (sometimes called a chipset) that interconnects and controls communications between system components.

The memory 111 may be a non-persistent memory, a persistent memory, or any combination thereof. Non-persistent memory typically includes high-speed random access memory, such as DRAM, SRAM, DDR RAM, ROM, EEPROM, flash memory, whereas the persistent memory typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. Persistent memory 112 optionally includes one or more storage devices remotely located from the CPU(s) 102. The persistent memory 112, and the non-volatile memory device(s) within the non-persistent memory 112, comprise non-transitory computer readable storage medium. Regardless of its specific implementation, the memory 111 comprises at least one non-transitory computer readable storage medium, and it stores thereon computer-executable executable instructions which can be in the form of programs, modules, and data structures.

In some embodiments, as shown in FIG. 1, the memory 111 stores the following programs, modules and data structures, or a subset thereof.
an operating system 116, which includes procedures for handling various basic system services and for performing hardware-dependent tasks;
an optional network communication module (or instructions) 118 for connecting the system 100 with other devices and/or to a communication network;
a reference module 120 for determining tumor fractions of subjects;
for a test subject 122, information comprising: a first dataset 124 including a plurality of bin values 126 for N bins of the genome of the subject comprising a bin count 128 (e.g., copy number count based on sequence reads obtained from the respective reference subject) for each respective bin in a plurality of bins (e.g., 1, 2, . . . , N), and a second dataset 130 including a set of allele frequencies 1 comprising support identified for each variant in a plurality of variants (e.g., alleles, 1, 2, . . . , M);
a reference model 140 that has been trained to determine tumor fraction of a test subject, where the reference model has been trained at least in part on a training dataset 142 including, for each reference subject 144 of a first plurality of reference subjects (subject 1, subject 2, . . . subject X), a set of bin values 146 for the respective reference subject comprising a bin count (e.g., copy number count based on sequence reads obtained from the respective reference subject) for each respective bin in a plurality of bins (e.g., 1, 2, . . . , N), a set of allele frequencies 148 comprising support identified for each variant in a plurality of variants (e.g., alleles 1, 2, . . . , M) for the respective subject, and an indication of tumor fraction (150-1, . . . ) of the respective subject.

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the memory 111 optionally stores a subset of the modules and data structures identified above. Furthermore, in some embodiments, the memory stores additional modules and data structures not described above. In some embodiments, one or more of the above identified elements is stored in a computer system, other than that of visualization system 100, that is addressable by visualization system 100 so that visualization system 100 may retrieve all or a portion of such data when needed.

Although FIG. 1 depicts a “system 100,” the figure is intended more as a functional description of the various features that may be present in computer systems than as a structural schematic of the implementations described herein. In practice, and as recognized by those of ordinary skill in the art, items shown separately could be combined and some items can be separate. Moreover, although FIG. 1 depicts certain data and modules in the memory 111 (which can be non-persistent or persistent memory), it should be appreciated that these data and modules, or portion(s) thereof, may be stored in more than one memory. For example, in some embodiments, at least the first dataset 122, the second dataset 124, the reference module 120, and the reference model 140 are stored in a remote storage device that can be a part of a cloud-based infrastructure. In some embodiments, at least the first dataset 122 and the second dataset 124 are stored on a cloud-based infrastructure. In some embodiments, the reference model 120 and the reference model 140 can also be stored in the remote storage device(s).

While a system in accordance with the present disclosure has been disclosed with reference to FIG. 1, methods in accordance with the present disclosure are now detailed. Any of the methods in accordance with embodiments of the present disclosure can make use of any of the assays, algorithms, or techniques, or combinations thereof, disclosed in U.S. Patent Publication No. US20180237863 and/or International Patent Publication No. WO2018081130, each of which is hereby incorporated herein by reference in its entirety, in order to determine a cancer condition in a test subject or a likelihood that the subject has the cancer condition.

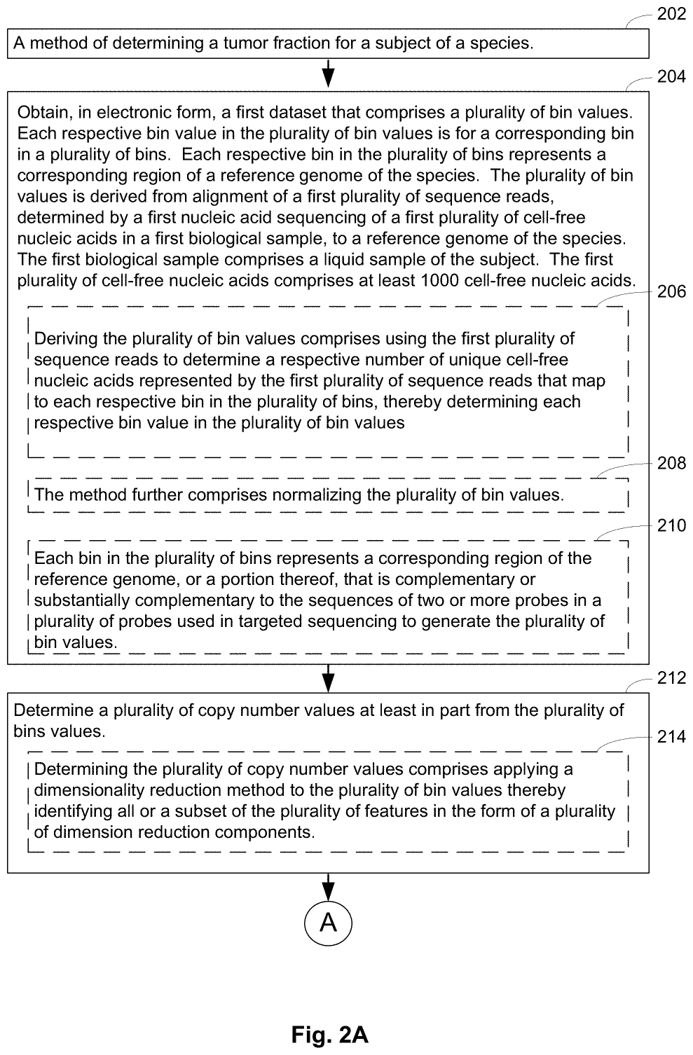

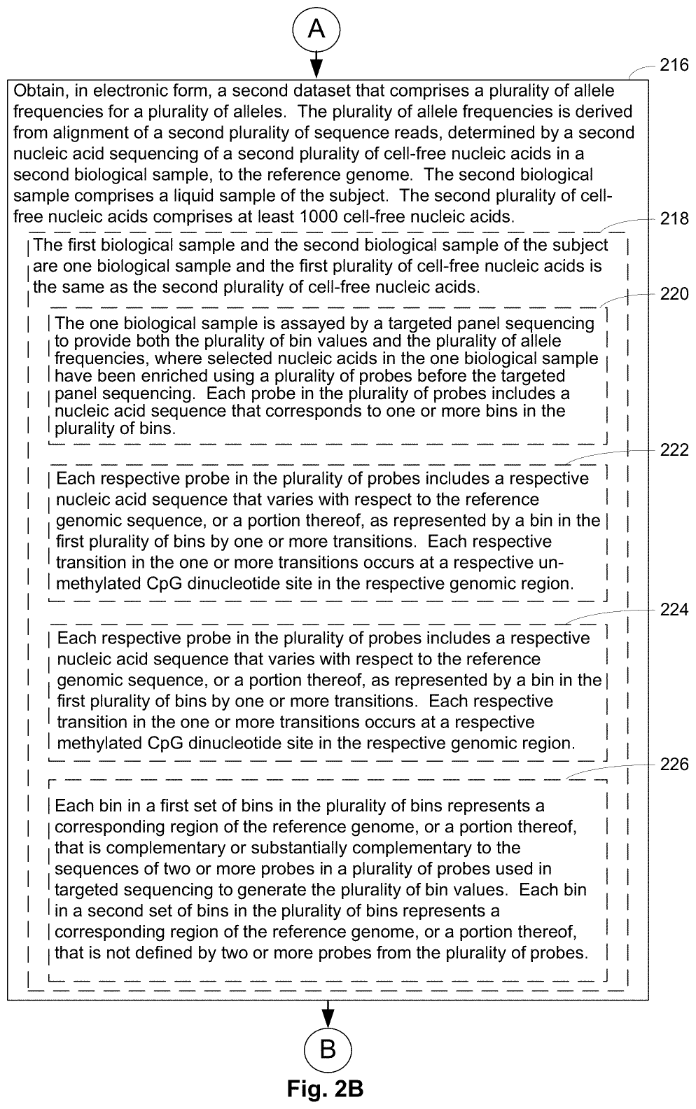

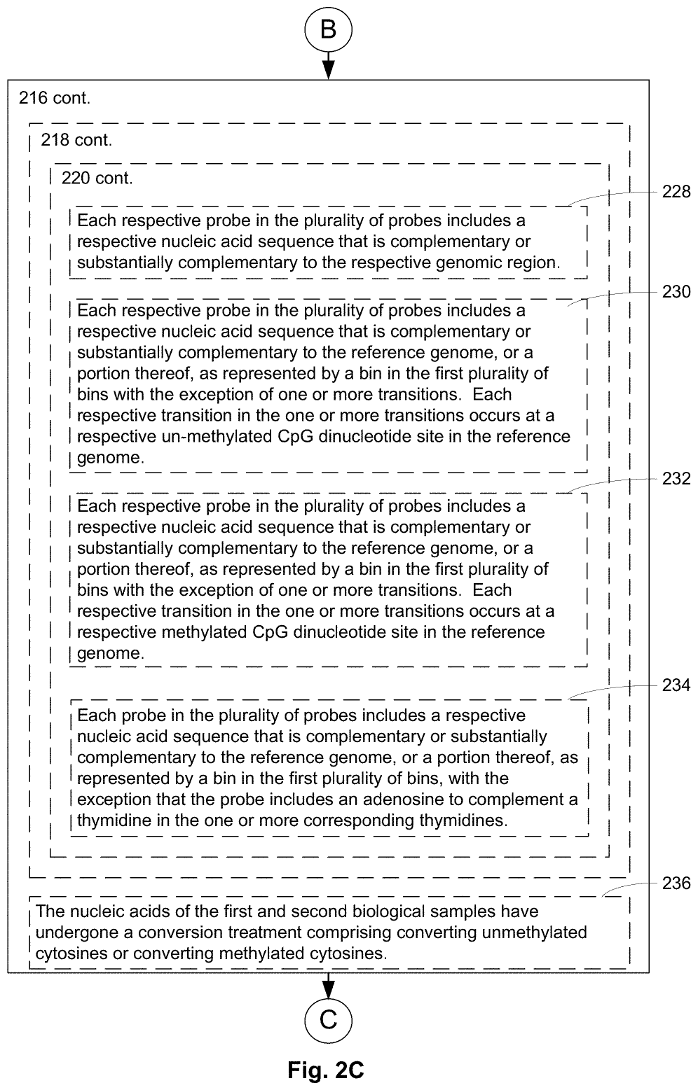

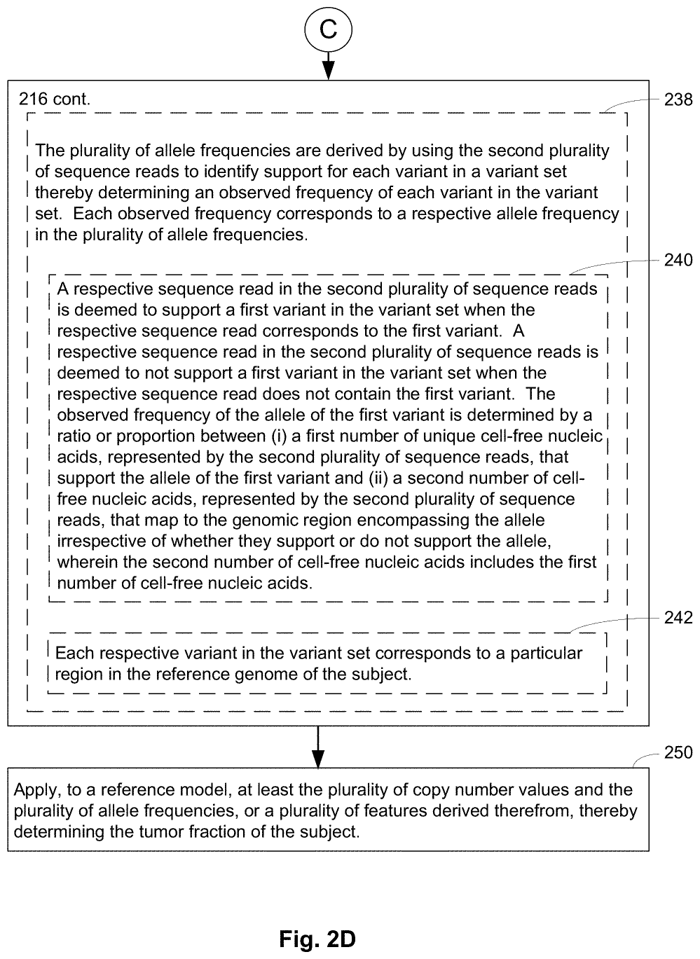

FIG. 2 illustrates an overview of the techniques in accordance with some embodiments of the present disclosure. In the described embodiments, a plurality of bin values and a plurality of allele frequencies are obtained for a subject. A plurality of copy number values is derived, at least in part, from the plurality of bin values. The plurality of copy number values and the plurality of allele frequencies (or a plurality of features derived therefrom) are applied to a reference model (e.g., a model trained as described below). The reference model, in response, determines the tumor fraction of the subject.

Block 202. Referring to block 202 of FIG. 2A, a method of determining a tumor fraction for a subject of a species is provided.

Block 204. Referring to block 204 of FIG. 2A, the method proceeds by obtaining, in electronic form, a first dataset that comprises a plurality of bin values. Each respective bin value in the plurality of bin values is for a corresponding bin in a plurality of bins. Each respective bin in the plurality of bins represents a corresponding region of a reference genome of the species. The plurality of bin values is derived from alignment of a first plurality of sequence reads, determined by a first nucleic acid sequencing of a first plurality of cell-free nucleic acids in a first biological sample, to a reference genome of the species. In some embodiments, the first biological sample comprises a liquid sample of the subject. In some embodiments, the first plurality of cell-free nucleic acids comprises at least 1000 cell-free nucleic acids. In some embodiments, alignment of each sequence read in the first plurality of sequence reads to a reference genome of the species is performed using a Smith-Waterman gapped alignment as implemented in, for example Arioc, or a Burrows-Wheeler transform as implemented in, for example Bowtie. Other suitable alignment programs includes, but are not limited to BarraCUDA, BBMap, BFAST, BigBWA, BLASTN, BLAT, BWA, BWA-PSSM, CASHX, to name a few. See, for example, Li and Durbin, 2009, “Fast and accurate short read alignment with Burrows-Wheeler transform,” Bioinformatics 25(14), 1754-1760; and Smith and Yun, 2017, “Evaluating alignment and variant-calling software for mutation identification in C. elegans by whole-genome sequencing,” PLOS ONE, doi.org/10.1371/journal.pone.0174446, each of which is hereby incorporated by reference.

In some embodiments, the first plurality of cell-free nucleic acids comprises at least 100, at least 500, at least 1000, at least 2000, at least 3000, at least 4000, at least 5000, at least 6000, at least 7000, at least 8000, at least 9000, at least 10,000, at least 20,000, at least 50,000, at least 100,000, or at least one million cell-free nucleic acids. In some embodiments such cell-free nucleic acids are aligned to the reference genome of the species.

In some embodiments, bin values are determined from methylation sequencing information (e.g., bin values correspond to ratios of abnormally methylated fragments versus fragments having a methylation status matching the methylation status for a healthy control group); and in some such embodiments, bin values are determined using methylation state vectors as described in Example 5 in PCT/US2020/034317, entitled “Systems And Methods For Determining Whether A Subject Has A Cancer Condition Using Transfer Learning,” filed May 22, 2020, which is hereby incorporated by reference. In the present disclosure, the section below entitled “Protocol for obtaining methylation information from sequence reads of fragments in a biological sample” provides one example of first nucleic acid sequencing method in which methylation information is derived from the sequence reads and used to determine bin values.

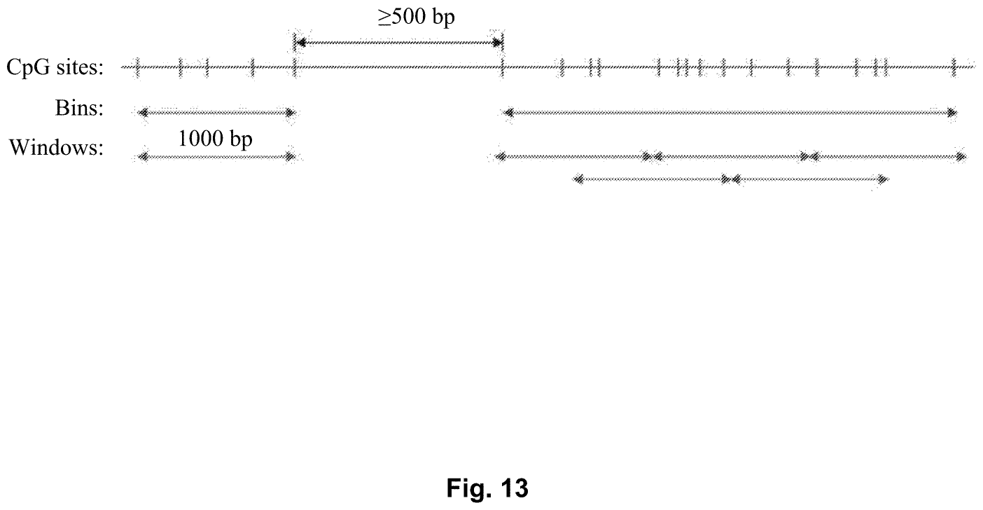

FIG. 13 is an illustration of bins of a reference genome, according to some embodiments of the present disclosure. A reference genome (or a subset of the reference genome) is partitioned in one or more stages, e.g., for use cases involving a targeted methylation assay (e.g., where the first dataset includes binned methylation data). For instance, in some embodiments, the reference genome is divided into bins (blocks) of CpG sites (e.g., each bin corresponds to a region of the reference genome that encompasses one or more CpG sites). In some such embodiments, each bin is defined when there is a separation between two adjacent CpG sites that exceeds a threshold, e.g., greater than 200 base pairs (bps), 300 bps, 400 bps, 500 bps, 600 bps, 700 bps, 800 bps, 900 bps, or 1,000 bps, among other values. Bins of a reference genome can vary in size of base pairs (e.g., bins within a plurality of bins can be different sizes). In the case where the first dataset is methylation data from targeted sequencing, a common size for bins is around 200 bps, with a range from about 30 bps to about 1000 bps or greater. In some embodiments, each bin is between 30 bps and 5000 bps. In some embodiments, when a respective bin in a plurality of bins is larger than a threshold size (e.g., 900 bps, 1000 bps, 1100 bps, etc.) the respective bin is subdivided into windows of a certain length, e.g., 500 bps, 600 bps, 700 bps, 800 bps, 900 bps, 1,000 bps, 1,100 bps, 1,200 bps, 1,300 bps, 1,400 bps, or 1,500 bps, among other values and each such window receives its own independent bin value. In other embodiments, the windows can be from 200 bps to 10 kilobase pairs (kbp), from 500 bp to 2 kbp, or about 1 kbp in length. Windows (e.g., that are adjacent) can overlap by a number of base pairs or a percentage of the length, e.g., 10%, 20%, 30%, 40%, 50%, or 60%, among other values. In embodiments, where a bin is divided into a plurality of windows, each feature extraction function of the present disclosure independently encodes a linear or nonlinear function of window values for each of the windows of the respective bin. In some embodiments, rather than dividing larger bins into windows, such larger bins are divided into smaller bins. In some embodiments, such smaller bins overlap each other while in other embodiments they do not overlap each other.

In some embodiments, each respective bin in the plurality of bins represents a non-overlapping corresponding region of the reference genome of the species.

In some embodiments, a respective bin in the plurality of bins overlaps a region corresponding to another bin in the plurality of bins. For example, in some embodiments, one or more bins in the plurality of bins overlap another adjacent bin (or bins) in the plurality of bins (e.g., two or more bins represent overlapping regions of the reference genome of the species).

In some embodiments of the present disclosure, the reference genome is the human genome. In some such embodiments, the human genome is divided into roughly 30 thousand bins. Then, certain of the bins are removed from consideration for the plurality of bins of the present disclosure using the methods disclosed in U.S. Patent Publication No. US 2019-0287649 A1, entitled “Method and System for Selecting, Managing, and Analyzing Data of High Dimensionality,” published Sep. 19, 2019, which is hereby incorporated by reference, to arrive at a subset of the 30,000 bins that is used for the plurality of bins, e.g., 23,000 bins. In such embodiments, each bin is roughly the same size, in terms of the amount of a human reference genome that corresponds to the bin.

In some embodiments, each bin value is a count of a number of cell-free nucleic acids from a biological sample that map to a bin. In some embodiments, this is determined through nucleic acid sequencing schemes that make use of a unique molecular identifier (UMI). That is, during the sequencing, each cell-free nucleic acid in a biological sample, and all the sequence reads that are derived from the cell-free nucleic acid, are assigned the same UMI. Thus, all the sequence reads that have the same UMI are considered to have been derived from a common cell-free nucleic acid (interchangeably referred to a “fragment”) and thus are bagged into a single consensus sequence for the common cell-free nucleic acid. See Smith et al., 2017, “UMI-tools: modeling sequencing errors in Unique Molecular Identifiers to improve quantification accuracy,” Genome Research 27(3), 491-499, which is hereby incorporated by reference, for sequencing schemes that make use of UMIs. The term “bin value” refers to any form of representation of the number of cell-free nucleic acids mapping to a given bin i. Such bin values can be in an un-normalized form (e.g., bvᵢ) or normalized form (e.g., bvᵢ*, bvᵢ**, bvᵢ***, bvᵢ****, etc.). The section below entitled “Determining bin values from counts of sequence reads” provides a description of an example method for determining bin values.

Referring to block 206, in some embodiments, deriving the plurality of bin values comprises using the first plurality of sequence reads to determine a respective number of unique cell-free nucleic acids represented by the first plurality of sequence reads that map to each respective bin in the plurality of bins, thereby determining each respective bin value in the plurality of bin values.

In some embodiments, a number of cell-free nucleic acids represented by sequence reads in the first plurality of sequence reads is determined for each bin the plurality of bins, for example as described in Example 5. In some embodiments, unique cell-free nucleic acids (e.g., used for determining bin values) are determined by bagging PCR duplicates of sequence reads that have the same barcode (e.g., a UMI or unique molecular identifier). In some embodiments, when a cell-free nucleic acid overlaps multiple bins, it is assigned (contributes to the count) in each bin it overlaps. In some embodiments, when a cell-free nucleic acid overlaps multiple bins, it is assigned (contributes to the count) of the bin it overlaps the most.

In some embodiments, the plurality of bins is constructed by dividing all or a portion of a reference genome (e.g., mammalian, human, etc.) into equally sized bins, where each bin represents a unique equally sized part of the reference genome. In some embodiments, the plurality of bins is constructed by dividing all or a portion of a reference genome (e.g., mammalian, human, etc.) into equally or unequally sized bins, where each bin represents a unique part of the reference genome.

In some embodiments, the plurality of bins is constructed by dividing all or a portion of a reference genome (e.g., mammalian, human, etc.) into equally or unequally sized bins, where each bin represents a corresponding part of the reference genome. In such embodiments, the corresponding part of the reference genome represented by one bin in the plurality of bins can overlap with the corresponding part of the reference genome represented by another bin in the plurality of bins. In some such embodiments, the plurality of bins is constructed by dividing all of a reference genome (e.g., mammalian, human, etc.) into equally or unequally sized bins, where each bin represents a corresponding overlapping or non-overlapping part of the reference genome. In some embodiments, the plurality of bins is constructed by dividing a portion of a reference genome (e.g., mammalian, human, etc.) into equally or unequally sized bins, where each bin represents an overlapping or non-overlapping part of the reference genome.

In some embodiments, the plurality of bins is constructed such that at least some of the regions of the human genome implicated in absence or presence of cancer (e.g., drawn from the regions identified in Examples 4, 7, 8 and/or 9) are represented by the plurality of bins whereas other regions of the reference genome are not represented by the bins.

Regardless of approach, each bin represents a unique part of the reference genome. In some embodiments, particularly when the bin values for such bins represent epigenetic features of methylation data obtained from targeted sequencing for the first dataset, such bins range in size between 30 bps and 5000 bps, between 30 bps and 4000 bps, between 30 bps and 3000 bps, between 30 bps and 2000 bps, between 30 bps and 1000 bps, or between 40 bps and 800 bps of the reference genome. In alternative embodiments, such bins range in size between 10,000 bps and 100,000 bps, between 20,000 bps and 300,000 bps, between 30,000 bps and 500,000 bps, between 40,000 bps and 1,000,000 bps between 50,000 bps and 5,000,000 bps, or between 100,000 bps and 25,000,000 bps of the reference genome.

In some embodiments, the portion of the reference genome is between 1 and 22 chromosomes of the reference genome, or at least 25 percent, at least 30 percent, at least 35 percent, at least 40 percent, at least 45 percent, at least 50 percent, at least 55 percent, at least 60 percent, at least 65 percent, at least 70 percent, at least 75 percent, at least 80 percent, at least 85 percent, at least 90 percent, at least 95 percent, or at least 99 percent of the reference genome. In some such embodiments, each bin represents between 10,000 bases and 100,000 bases, between 20,000 bases and 300,000 bases, between 30,000 bases and 500,000 bases, between 40,000 bases and 1,000,000 bases between 50,000 bases and 5,000,000 bases, or between 100,000 bases and 25,000,000 bases of the reference genome.

In some embodiments, each of the bins represents a specific site of a reference genome that has been identified as being associated with cancer.

In some embodiments, each of the bins represents a specific region of a reference genome that has been identified as being associated with cancer through cancer- and/or tissue-specific methylation patterns in cfDNA relative to non-cancer controls. For example, the section below entitled “Example bins for methylation embodiments” discloses 103,456 such distinct regions. Examples 7, 8, and 9 also disclose a number of distinct regions. In some embodiments, there is a one to one correspondence between such bins and these regions. In other words, in such embodiments, each bin encompasses a single unique one of the regions identified in Examples 4, 7, 8 and/or 9. In some such embodiments, each bin ranges in size between 30 bps and 5000 bps, between 30 bps and 4000 bps, between 30 bps and 3000 bps, between 30 bps and 2000 bps, between 30 bps and 1000 bps, or between 30 bps and 750 bps. In some embodiments, in the case where the regions used are drawn from Examples 4, 7, 8, and/or 9, each bin includes between 1 and 590 cytosine-guanine dinucleotides (CpGs). In some embodiments, some of the bins represent regions that are hypomethylated in the cancer-state relative to the cancer-free normal state. In some embodiments, some of the bins represent regions that are hypermethylated in the cancer-state relative to the cancer-free normal state. In some embodiments, the plurality of bins used collectively encompass at least 1000, at least 2000, at least 3000, at least 4000, at least 5000, at least 6000, at least 7000, at least 8000, at least 9000, at least 10000, at least 25000, at least 30000, at least 40000, or at least 50000 of the regions identified in Examples 4, 7, 8, and/or 9 with each bin in the plurality of bins representing a different unique region in the plurality of regions identified in Examples 4, 7, 8, and/or 9. In such embodiments, the bin value for each bin is based on a number of nucleic acid fragments, as ascertained from the corresponding first plurality of sequence reads acquired from a biological sample of a respective subject that map to the respective bin.

In some embodiments, the plurality of bins is derived from the sequences disclosed in Examples the sections below entitled “Example bins for methylation embodiments,” “Select human genomic regions used for bins,” Additional select human genomic regions used for bins, and/or “Additional Select human genomic regions used for bins.” In some such embodiments, adjacent and overlapping targets (genomic sequence targeted by a probe to a region disclosed in the sections below entitled “Example bins for methylation embodiments,” “Select human genomic regions used for bins,” Additional select human genomic regions used for bins, and/or “Additional Select human genomic regions used for bins”) are merged into contiguous genomic regions. In some embodiments, each of the resulting regions is used as-is as a corresponding bin in the plurality of bins if smaller than a threshold number of base pairs (e.g., 1000 base pairs), or else subdivided into sub-regions (e.g., 1000 base pair regions). It will be appreciated that the present disclosure is not limited to bins having 1000 base pair regions and that any positive integer value between 100 base pairs and 10 million base pairs can be used to define the bins. Moreover, it will be appreciated that, rather than dividing a genome by base pair values to form bins, the genome can be divided into bins based on blocks of CpG sites, such as between 1 and 1000 CpG sites per bin (e.g., rather than by explicitly considering base pair lengths for such bins). In some embodiments, the bins are arranged so that consecutive bins overlap by a certain number of base pairs (e.g., in the case of 1000 base pair bins, by, for example, overlapping by 500 base pairs) which may or may not represent a certain number of CpG sites. In some embodiments, each bin ranges in size between 30 bps and 5000 bps, between 30 bps and 4000 bps, between 30 bps and 3000 bps, between 30 bps and 2000 bps, between 30 bps and 1000 bps, or between 30 bps and 750 bps.

In some embodiments, the plurality of bins is derived such that each bin encompasses one, two, three, four, five, six, seven, or eight probes described in the section below entitled “Cancer assay probes and panels.” In some such embodiments, adjacent and overlapping targets (genomic sequence targeted by a probe in the section below entitled “Cancer assay probes and panels”) are merged into contiguous genomic regions. In some embodiments, each of the resulting regions is used as-is as a corresponding bin in the plurality of bins if smaller than a threshold number of base pairs (e.g., 1000 base pairs), or else subdivided into sub-regions (e.g., 1000 base pair regions). It will be appreciated that the present disclosure is not limited to bins having 1000 base pair regions and that any positive integer value between 100 base pairs and 10 million base pairs can be used to define the bins. In some embodiments, the bins are arranged so that consecutive bins overlap by a certain number of base pairs (e.g., in the case of 1000 base pair bins, by, for example, overlapping by 500 base pairs). In some such embodiments, each bin ranges in size between 30 bps and 5000 bps, between 30 bps and 4000 bps, between 30 bps and 3000 bps, between 30 bps and 2000 bps, between 30 bps and 1000 bps, or between 30 bps and 750 bps.

In some embodiments, the plurality of bins is derived such that each bin encompasses a region of the genome described in the section below entitled “Example bins for methylation embodiments.” In some such embodiments, each bin ranges in size between 30 bps and 5000 bps, between 30 bps and 4000 bps, between 30 bps and 3000 bps, between 30 bps and 2000 bps, between 30 bps and 1000 bps, or between 30 bps and 750 bps.

In some embodiments, the plurality of bins is derived such that each bin encompasses a region of the genome described in the section below entitled “Select human genomic regions used for bins.” In some such embodiments, each bin ranges in size between 30 bps and 5000 bps, between 30 bps and 4000 bps, between 30 bps and 3000 bps, between 30 bps and 2000 bps, between 30 bps and 1000 bps, or between 30 bps and 750 bps.

In some embodiments, the plurality of bins is derived such that each bin encompasses a region of the genome described in the section below entitled “Additional select human genomic regions used for bins.” In some such embodiments, each bin ranges in size between 30 bps and 5000 bps, between 30 bps and 4000 bps, between 30 bps and 3000 bps, between 30 bps and 2000 bps, between 30 bps and 1000 bps, or between 30 bps and 750 bps.

In some embodiments, the plurality of bins is derived such that each bin encompasses a region of the genome described in the section below entitled “Additional Select human genomic regions used for bins.” In some such embodiments, each bin ranges in size between 30 bps and 5000 bps, between 30 bps and 4000 bps, between 30 bps and 3000 bps, between 30 bps and 2000 bps, between 30 bps and 1000 bps, or between 30 bps and 750 bps.

In some embodiments, the plurality of bins is derived from any combination of the bins disclosed in the sections entitled Example bins for methylation embodiments, “Select human genomic regions used for bins,” “Additional select human genomic regions used for bins,” or “Additional Select human genomic regions used for bins.” In some such embodiments, each bin ranges in size between 30 bps and 5000 bps, between 30 bps and 4000 bps, between 30 bps and 3000 bps, between 30 bps and 2000 bps, between 30 bps and 1000 bps, or between 30 bps and 750 bps.

In some embodiments, each bin represents all or a portion of an enhancer, promoter, 5′ UTR, exon, exon/inhibitor boundary, intron, intron/exon boundary, 3′ UTR region, CpG shelf, CpG shore, or CpG island in a reference genome. See, for example, Cavalcante and Santor, 2017, “annotatr: genomic regions in context,” Bioinformatics 33(15) 2381-2383, for suitable definitions of such regions and where such annotations are documented for a number of different species.

In some embodiments, a reference genome (or a subset of the reference genome) is partitioned in one or more stages, e.g., for use cases involving a targeted methylation assay. For instance, the reference genome is separated into blocks (bins) of CpG sites. As used herein, in this context, the terms “bins” and “blocks” are used interchangeably. In some such embodiments, each bin (block) is defined when there is a separation between two adjacent CpG sites that exceeds a threshold, e.g., greater than 200 base pairs (bp), 300 bp, 400 bp, 500 bp, 600 bp, 700 bp, 800 bp, 900 bp, or 1,000 bp, among other values. Thus, bins (blocks) in such embodiments can vary in size of base pairs. For each respective bin (block), the respective bin is divided into windows of a certain length, e.g., 500 bp, 600 bp, 700 bp, 800 bp, 900 bp, 1,000 bp, 1,100 bp, 1,200 bp, 1,300 bp, 1,400 bp, or 1,500 bp, among other values. In other embodiments, the windows can be from 200 bp to 10 kilobase pairs (kbp), from 500 bp to 2 kbp, or about 1 kbp in length. Windows (e.g., that are adjacent) can overlap by a number of base pairs or a percentage of the length, e.g., 10%, 20%, 30%, 40%, 50%, or 60%, among other values.

Sequence reads derived from cell-free nucleic acids are then analyzed using a windowing process in some embodiments. In particular, a sequence processor scans through the bins window-by-window and reads cell-free nucleic acids within each window. Such windows of bins are illustrated in FIG. 13. In some embodiments, the cell-free nucleic acids originate from tissue and/or tumors. By partitioning the reference genome (e.g., using bins and windows), computational parallelization is facilitated. Moreover, computational resources, to process a reference genome by targeting the sections of base pairs that include CpG sites, while skipping other sections that do not include CpG sites, are reduced. See, for example, U.S. patent application Ser. No. 15/931,022, entitled “Model Based Featurization and Classification,” filed May 13, 2020, which is hereby incorporated by reference.

In some embodiments, each respective bin value in the first plurality of bin values for a corresponding bin in the plurality of bins for the test subject is determined by identifying the number of cell-free nucleic acids represented in the first plurality of sequence reads obtained from the biological sample of the subject, that map to the genomic region represented by the corresponding bin.

In some embodiments, each respective bin value in the plurality of bin values is a measure of a frequency of abnormally methylated cell-free nucleic acids (e.g., cell-free nucleic acids including one or more abnormally methylated CpG sites) represented by the first plurality of sequence reads that map to the genomic region represented by the corresponding bin.

In some embodiments, each respective bin value in the plurality of bin values is determined from a methylation state vector derived from the first plurality of sequence reads that map to the genomic region represented by the corresponding bin. There are various ways to determine whether a specific cell-free nucleic acid (fragment) includes one or more abnormally methylated CpG sites. For example, U.S. patent application Ser. No. 16/719,902, entitled “Systems and Methods for Estimating Cell Source Fractions using Methylation Information,” filed Dec. 18, 2019, which is hereby incorporated by reference in its entirety, discloses methods for determining whether cell-free nucleic acids are abnormally methylated (e.g., by comparing methylation states for each respective cell-free nucleic acid to a reference dataset of methylation states—where the reference dataset is determined from the methylation states observed in a cohort of healthy reference subjects).

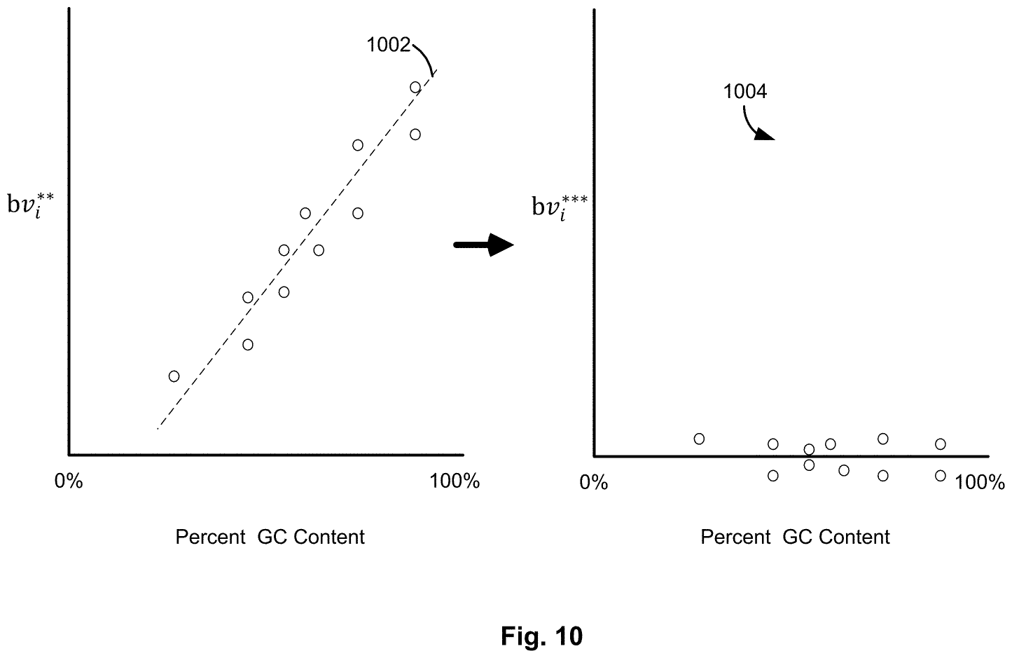

Referring to block 208, in some embodiments, the method further comprises normalizing the plurality of bin values. In some embodiments, each bin value is normalized from a respective number of cell-free nucleic acids represented by sequence reads for the corresponding bin. In some embodiments, the normalization is performed by correction of GC biases (e.g., as described below in the section entitled Determining bin values from counts of sequence reads, and as illustrated in FIG. 10). In some embodiments, the normalization is performed by correction of biases due to PCR over-amplification (e.g., as described below in the section entitled Determining bin values from counts of sequence reads).

In some embodiments, sequence reads obtained from a biological sample of a subject are normalized relative to a reference set (e.g., as obtained from a plurality of reference subjects—such as a control cohort of healthy subjects). U.S. Patent Publication No. 2019-0287649, entitled “Method and System for Selecting, Managing, and Analyzing Data of High Dimensionality,” published Sep. 19, 2019, which is hereby incorporated by reference herein in its entirety, discloses multiple methods of normalization. In some embodiments, bin counts are normalized against an overall average of bin counts for a plurality of health subjects (e.g., a control group). In some embodiments, bin counts are normalized against a per-bin average of bin counts for the plurality of health subjects.

In some embodiments, sequence reads of a subject are normalized against an overall average count of sequence reads that is determined from a group of subjects (e.g., a group of n baseline healthy subjects). For example, an overall average R̅e̅a̅d̅ can be computed based on the average of every subject in the baseline control group, using the equation:

R̅e̅a̅d̅=Σᵢ₌₁ⁱ⁼ⁿR̅e̅a̅d̅ₖ̅/n

Here, R̅e̅a̅d̅ₗ̅ is the average of a baseline healthy subject across different genomic regions (e.g., across a plurality of bins, where integer k denotes a subject and is 1 through n. R̅e̅a̅d̅ₖ̅ can be determined, for example, using the equation above.

In some embodiments, the overall average R̅e̅a̅d̅ is used to normalize the number of sequence reads bound to a particular region (x) for any future subject, for example, using the equation:

NormalizedRead=R̅e̅a̅d̅×SizeRegion(x)=wₓ×ActualRead(x),

where ActualRead(x) is the actual number of sequence reads for the subject that are aligned to region x (e.g., a bin or other genomic region), and wₓ is a weight assigned to the region to normalize the sequence reads to an expected value that can be obtained using an overall average.

In some embodiments, sequence reads corresponding to a particular region (e.g., bin) are normalized against an averaged number of sequence reads for the same region across a group of healthy subjects (e.g., baseline healthy subjects). As an illustration, the sequence reads for region (j) for a subject k can be represented as Readₖ^{j}, where a subject k is an integer from 1 to n. The average number of sequence reads for region (j) cross all subjects can be computed based on the following:

R̅e̅a̅d̅^̅{̅J̅}̅=Σᵢ₌₁ⁱ⁼ⁿReadᵢ^{j}/n.

Using this cross-subject average as a reference, the sequence reads for region (j) for a subject can be computed as:

NormalizedRead=R̅e̅a̅d̅^̅{̅J̅}̅=wⱼ×ActualRead(j),

where ActualRead(j) is the actual number of sequence reads aligned to region j, and wⱼ is a weight assigned to the region to normalize the sequence reads to an expected value that can be obtained using average read R̅e̅a̅d̅^̅{̅J̅}̅.

Another option is to normalize bin counts based on average bin counts for a particular subject (e.g., not using a control group).

In some embodiments, each bin value indicates a respective copy number instability (CNI) or copy number score for the corresponding bin. See Zhou et al. 2018 Bioinformatics 34(14), 2349-2355, which is hereby incorporated by reference, for an example method of how copy number score (i.e., here Z-score) may be calculated from bin count or bin value. In some embodiments, a bin value is in the form of a B-score, which is described, for example, in U.S. Patent Publication No. 2019-0287649, entitled “Method and System for Selecting, Managing, and Analyzing Data of High Dimensionality,” published Sep. 19, 2019, which is hereby incorporated by reference herein in its entirety.

In some embodiments, where the sequencing assay is whole genome bisulfite sequencing, methylation state vectors are determined as disclosed in U.S. patent application Ser. No. 16/352,602, entitled “Anomalous Fragment Detection and Classification,” filed Mar. 13, 2019, or in accordance with any of the techniques disclosed in U.S. patent application Ser. No. 15/931,022, entitled “Model-Based Featurization and Classification,” filed May 13, 2020, each of which is hereby incorporated by reference. In such embodiments, a bin value reflects a number of fragments as represented by sequence reads that have a predetermined methylation state and that map onto the region of the reference genome corresponding to the respective bin. As an example, the bin value reflects methylation states based on the presence of CpG sites over a given length of nucleotide sequence.

In some embodiments, not all nucleic acid fragments recovered from the first biological sample are used to determine bin values. This is due to the fact that nucleic acid fragments (cell-free nucleic acids) vary in terms of information content, and in some embodiments only those nucleic acid fragments with the desired information content are retained for bin value determination (e.g., fragments that do not provide relevant information are discarded). In some embodiments, bin values are determined from nucleic acid fragments that satisfy one or more filter conditions in a plurality of filtering conditions (where each filter condition evaluates the information content of the fragments). Multiple filtering methods are described, for example, in detail in International Patent Application No. PCT/US2020/034317, entitled “Systems and Methods for Determining Whether a Subject has a Cancer Condition Using Transfer Learning,” filed May 22, 2020, which is hereby incorporated by reference. Non-limiting examples of filter conditions are provided below.

P-value filtering based on methylation vectors. In some embodiments, a filter condition in the plurality of filter conditions is a requirement that each cell-free nucleic acid in the plurality of cell-free nucleic acids used as part of determining bin counts have a corresponding p-value that is below a threshold value, where the p-value is determined by p-value filtering as described Example 5 in International Patent Application No. PCT/US2020/034317. The goal of such a filter condition is to accept and use anomalously methylated cell-free nucleic acids for the determination of bin values based on their corresponding methylation state vectors. For example, for each cell-free nucleic acid (fragment) in a sample, a determination is made as to whether the fragment is anomalously methylated (e.g., via analysis of sequence reads derived therefrom), relative to an expected methylation state vector using the methylation state vector corresponding to the fragment (e.g., where the expected methylation state vector is determined from sequence analysis of a cohort (plurality) of healthy subjects). The generation of methylation state vectors for such cell-free nucleic acids (fragments) is disclosed, for example, in the section below entitled “Protocol for obtaining methylation information from sequence reads of fragments in a biological sample.” In some embodiments, the threshold value is 0.01 (e.g., p must be <0.01 in such embodiments). In some embodiments, the threshold value is 0.001, 0.005, 0.01, 0.015, 0.02, 0.05, or 0.10. In some embodiments, the threshold value is between 0.0001 and 0.20. In such embodiments, only those cell-free nucleic acids that have a p-value below the threshold value contribute to bin count. For example, in some embodiments, the plurality of cell-free nucleic acids is filtered by removing from the plurality of cell-free nucleic acids each respective cell-free nucleic acid whose corresponding methylation pattern (e.g. methylation state vector) across a corresponding plurality of CpG sites in the respective fragment has a p-value that fails to satisfy a p-value threshold.

Minimum bag-size. In some embodiments, a filter condition in the plurality of filter conditions is a requirement that each cell-free nucleic acid (fragment) have a bag-size greater than a threshold integer. In other words, that each cell-free nucleic acid be represented by more than the threshold integer of sequence reads in the first plurality of sequence reads. For example, in the case where the threshold integer is one, each cell-free nucleic acid must be represented by more than one sequence read in the first plurality of sequence reads. In some embodiments, the threshold integer is 1, 2, 3, 4, 5, 6, 7, 8, 9, 10, or an integer between 10 and 100.

Minimum number of CpG sites. In some embodiments, a filter condition in the plurality of filter conditions is a requirement that each cell-free nucleic acid covers a first threshold number of CpG sites and be less than a second threshold length in terms of base pairs. For example, in the case where the first threshold is 1 CpG site and the second threshold 1000 base pairs, each cell-free nucleic acid must cover more than one CpG site and be less than 1000 base pairs in length. In some embodiments, each cell-free nucleic acid must cover at least 1, 2, 3, 4, 5, 6, 7, 8, 9, 10, 11, 12, 13, 14, 15, 16, 17, 18, 19, or 20 CpG sites (e.g., within a particular nucleic acid length). In some embodiments, each cell-free nucleic acid must be less than 500, 1000, 2000, 3000, or 4000 contiguous base pairs in length. In other words for example, in some embodiments, the filter condition in the plurality of filter conditions requires that each cell-free nucleic acid that contributes to a bin count include at least 1 CpG site, at least 2 CpG sites, at least 3 CpG sites, at least 4 CpG sites, at least 5 CpG sites, at least 6 CpG sites, at least 7 CpG sites, at least 8 CpG sites, at least 9 CpG sites, at least 10 CpG sites, at least 11 CpG sites, at least 12 CpG sites, at least 13 CpG sites, at least 14 CpG sites, or at least 15 CpG sites within less than 500 contiguous nucleotides of the reference genome.

Hypermethylation or Hypomethylation. In some embodiments, a filter condition in the plurality of filter conditions is a requirement that each fragment is hypermethylated. In some embodiments, a filter condition in the plurality of filter conditions is a requirement that each cell-free nucleic acid is hypomethylated. In some embodiments, the filter condition is bin dependent. For instance, International Patent Publication No. WO2019/195268, entitled “Methylation Markers and Targeted Methylation Probe Panels,” filed Apr. 2, 2019, which is hereby incorporated by reference, discloses a number of regions of the human genome that have a hypermethylated state that is associated with one or more cancer conditions as well as a number of regions of the human genome that have a hypomethylated that is associated with one or more cancer conditions. Accordingly, in some embodiments of the present disclosure one or more bins in the plurality of bins each represent a corresponding genomic region in the regions disclosed in WO2019/19528 and the filter condition in the plurality of filter conditions (a) requires selection of cell-free nucleic acids that are hypermethylated when selecting cell-free nucleic acids that map to a bin representing a region of the human genome that has a hypermethylated state that is associated with one or more cancer conditions of CpG sites as indicated by WO2019/195268 and (b) requires selection of cell-free nucleic acids that are hypomethylated when selecting fragments that map to a bin representing a region of the human genome that has a hypomethylated state that is associated with one or more cancer conditions of CpG sites as indicated by WO2019/195268.

In some embodiments, the plurality of filter conditions requires the p-value threshold is satisfied and that the cell-free nucleic acid is hypermethylated. In some embodiments, the plurality of filter conditions requires the p-value threshold is satisfied and that the cell-free nucleic acid is hypomethylated. In some embodiments, the plurality of filter conditions is different for each bin. For instance, for one bin in the plurality of bins, the plurality of filter conditions require the p-value threshold is satisfied and that the cell-free nucleic acid is hypomethylated, while for a second bin in the plurality of bins, the plurality of filter conditions require the p-value threshold is satisfied and that the cell-free nucleic acid is hypermethylated.

Cancer condition. In some embodiments, a filter condition in the plurality of filter conditions is a requirement that each cell-free nucleic acid satisfy a cancer state threshold (e.g., that each cell-free nucleic acid have a probability above a predefined threshold of being associated with a respective cancer condition). In some embodiments, each cancer condition has a different respective predefined threshold. For example, as described in U.S. Patent Application No. 63/003,087, entitled Systems and Methods for Using Neural Networks to Determine a Cancer State, filed on Mar. 31, 2020, which is hereby incorporated by reference in its entirety, a trained neural network (e.g., trained on a plurality of reference subjects) is used to determine cancer probabilities for each genomic region (e.g., bin).

In some such embodiments, for each respective bin in the plurality of bins, for each respective cell-free nucleic acid in the plurality of cell-free nucleic acids that map to the respective bin, a corresponding trained neural network computes a prediction value that is the probability that the cell-free nucleic acid is associated with a cancer condition (e.g., cancer) based on the methylation pattern of the respective cell-free nucleic acid. Thus, in some such embodiments, the methylation pattern of the respective cell-free nucleic acid is scored using the trained neural network, where the score outputted by the trained neural network comprises the probability that the cell-free nucleic acid has the cancer state and/or a calculation based on the probability that the cell-free nucleic acid is associated with the cancer state

$$( e . g . , log ( \frac{P ( cancer state )}{P ( noncancer state )} ) ) .$$

The respective cell-free nucleic acid is subsequently tallied (e.g., contributes to bin count) if the resulting score satisfies the condition defined above (e.g., a probability that is above a fixed value threshold). The respective cell-free nucleic acid is subsequently not tallied (e.g., does not contribute to bin count) if the resulting score does not satisfy the condition defined above (e.g., a probability that is below a fixed value threshold). Then, for each respective bin in the plurality of bins, the respective bin value is the tallied count of all the cell-free nucleic acids that map to the respective bin and that satisfy the condition.

In some such embodiments, the threshold value is positive or negative. In some embodiments, the threshold value is between 0.1 and 1, between 1 and 5, between 5 and 10, between 10 and 50, between 50 and 100, or greater than 100. In some embodiments, the threshold value is between −0.1 and −1, between −1 and −5, between −5 and −10, between −10 and −50, between −50 and −100, or less than −100. In some embodiments, the threshold value is zero.

In some embodiments, each bin has a respective threshold for each respective cancer condition (e.g., a respective subset of bins is associated with each cancer condition).

In some embodiments, any combination of the disclosed filter conditions is imposed. In some embodiments, each bin value is a number of cell-free nucleic acids whose methylation patterns satisfy one or more filter conditions disclosed herein.

Referring to block 210, in some embodiments, the corresponding region of the reference genome, or a portion thereof, for each respective bin in the plurality of bins is complementary or substantially complementary to the sequences of two or more probes in a plurality of probes used in a targeted nucleic acid sequencing to generate the plurality of bin values. In some embodiments, such mapping to genomic regions allows some mismatching. In some embodiments, such mapping is performed using a Smith-Waterman gapped alignment as implemented in, for example Arioc, or a Burrows-Wheeler transform as implemented in, for example Bowtie. Other suitable alignment programs includes, but are not limited to BarraCUDA, BBMap, BFAST, BigBWA, BLASTN, BLAT, BWA, BWA-PSSM, CASHX, to name a few. See, for example, Langmead and Salzberg, 2012, Nat Methods 9, pp. 357-359; Li and Durbin, 2009, “Fast and accurate short read alignment with Burrows-Wheeler transform,” Bioinformatics 25(14), 1754-1760; and Smith and Yun, 2017, “Evaluating alignment and variant-calling software for mutation identification in C. elegans by whole-genome sequencing,” PLOS ONE, doi.org/10.1371/journal.pone.0174446, each of which is hereby incorporated by reference.

In some embodiments, genomic regions with high variability or low mappability are excluded from bin representation in the plurality of bins, for example, using the methods disclosed in Jensen et al, 2013, PLoS One 8; e57381. See also, Li and Freudenberg, 2014, Front. Genet. 5, p. 318, for analysis of mappability.

In some embodiments, each bin in the plurality of bins comprises at least 100 nucleic acid residues, at least 500 nucleic acid residues, at least 1000 nucleic acid residues, at least 2500 nucleic acid residues, at least 5000 nucleic acid residues, at least 10,000 nucleic acid residues, at least 25,000 nucleic acid residues, at least 50,000 nucleic acid residues, at least 100,000 nucleic acid residues, at least 250,000 nucleic acid residues, or at least at least 500,000 nucleic acid residues. In some embodiments, the plurality of bins comprises at least 50 bins, at least 100 bins, at least 250 bins, at least 500 bins, at least 1000 bins, at least 2500 bins, at least 3000 bins, at least 5000 bins, at least 10,000 bins, at least 200,000 bins, at least 300,000 bins, or at least 500,000 bins. In some embodiments, each bin is at least 100 Kb in length.

In some embodiments, each bin in the plurality of bins has a corresponding buffer region, where each respective buffer region comprises at least 10 nucleic acid residues, at least 50 nucleic acid residues, at least 100 nucleic acid residues, at least 150 nucleic acid residues, at least 200 nucleic acid residues, at least 250 nucleic acid residues, at least 500 nucleic acid residues, or at least 1000 nucleic acid residues.

In some embodiments, each respective bin in the plurality of bins represents a different portion of the genome of a reference genome for the species. The bins can have the same or different sizes (e.g., as illustrated in FIG. 13). In some embodiments, each respective bin in the plurality of bins represents a different non-overlapping portion of the genome of the reference genome for the species.

Block 212. Referring to block 212 of FIG. 2A, the method proceeds by determining a plurality of copy number values at least in part from the plurality of bins values.

In some embodiments, the sequence reads are corrected for background copy number. For instance, sequence reads that arise from chromosomes or portions of chromosomes that are duplicated in the subject are corrected for this duplication. This can be done either by normalizing before running this inference, or allowing for more than one value of first cell source fraction. Allowing for more than one first cell source fraction also enables assessment of heterogeneity within a test subject. As such, in some embodiments, the assumption that each sequence read represents an independent observation is corrected for background copy number. See e.g., Devonshire et al. 2014 Anal Bioanal Chem. 406(26): 6499-6512. In some embodiments, copy number determination is performed as described in U.S. patent application Ser. No. 16/816,918, filed Mar. 12, 2020, entitled “Systems and Methods for Enriching for Cancer-derived Fragments Using Fragment Size,” which is hereby incorporated by reference.

For instance, in some embodiments, each copy number is determined based on gene abundance level, e.g., the relative copy number of a predefined set of genes. In some embodiments, the predefined set of genes are selected based on evaluation of copy number variation across a plurality of cancer patients to identify genes for which copy number is informative of a tumor fraction. In some embodiments, each copy number is determined based on a genome-wide analysis of gene level (e.g., the relative copy number of each gene in the reference genome).

Further, in some embodiments, each respective copy number is determined from a corresponding bin value of a subset of the plurality of bins, as opposed to determining copy number from an overall metric for copy number variation across the genome as a whole. In some embodiments, the plurality of bins covers less than the entire reference genome, e.g., the plurality of bins is a subset of a larger set of bins spanning the entire reference genome. In some embodiments, the subset of bins is selected based on evaluation of copy number variation across a plurality of cancer patients to identify bins for which copy number is informative of a relevant cancer status of the subject, e.g., the presence or absence of cancer, a type of cancer, a stage of cancer, a prognosis for a cancer, or a therapeutic prediction for a cancer. One method for selecting such bins is disclosed in U.S. Patent Publication No. US 2019-0287649 A1, entitled “Method and System for Selecting, Managing, and Analyzing Data of High Dimensionality,” published Sep. 19, 2019, which is hereby incorporated by reference.

Referring to block 214, in some embodiments, determining the plurality of copy number values comprises applying a dimensionality reduction method (e.g., such as principal component analysis (PCA)) to the plurality of bin values, thereby identifying all or a subset of the plurality of features in the form of a plurality of dimension reduction components (e.g., principal components derived from the principal component analysis of the plurality of bin values). In some embodiments, the dimension reduction algorithm is a linear dimension reduction algorithm or a non-linear dimension reduction algorithm. In some embodiments, the dimension reduction algorithm is principal component analysis algorithm, a factor analysis algorithm, Sammon mapping, curvilinear components analysis, a stochastic neighbor embedding (SNE) algorithm, an Isomap algorithm, a maximum variance unfolding algorithm, a locally linear embedding algorithm, a t-SNE algorithm, a non-negative matrix factorization algorithm, a kernel principal component analysis algorithm, a graph-based kernel principal component analysis algorithm, a linear discriminant analysis algorithm, a generalized discriminant analysis algorithm, a uniform manifold approximation and projection (UMAP) algorithm, a LargeVis algorithm, a Laplacian Eigenmap algorithm, or a Fisher's linear discriminant analysis algorithm. See, for example, Fodor, 2002, “A survey of dimension reduction techniques,” Center for Applied Scientific Computing, Lawrence Livermore National, Technical Report UCRL-ID-148494; Cunningham, 2007, “Dimension Reduction,” University College Dublin, Technical Report UCD-CSI-2007-7, Zahorian et al., 2011, “Nonlinear Dimensionality Reduction Methods for Use with Automatic Speech Recognition,” Speech Technologies. doi:10.5772/16863. ISBN 978-953-307-996-7; and Lakshmi et al. (18 Aug. 2016). 2016 IEEE 6th International Conference on Advanced Computing (IACC). pp. 31-34. doi:10.1109/IACC.2016.16, ISBN 978-1-4673-8286-1, each of which is hereby incorporated by reference. Further examples of feature extraction methods for use in dimensionality reduction are described in more detail below.

Block 216. Referring to block 216 of FIG. 2B, the method continues by obtaining, in electronic form, a second dataset that comprises a plurality of allele frequencies for a plurality of alleles. The plurality of allele frequencies is derived from alignment of a second plurality of sequence reads, determined by a second nucleic acid sequencing of a second plurality of cell-free nucleic acids in a second biological sample, to the reference genome. In some embodiments, the second biological sample comprises a liquid sample of the subject. In some embodiments, the second plurality of cell-free nucleic acids comprises at least 1000 cell-free nucleic acids.

In some embodiments, the second plurality of cell-free nucleic acids comprises at least 1000, at least 2000, at least 3000, at least 4000, at least 5000, at least 6000, at least 7000, at least 8000, at least 9000, at least 10,000, at least 20,000, at least 50,000, or at least 100,000 cell-free nucleic acids.

In some embodiments, the method further comprises using the second plurality of sequence reads to identify support for an allele for a variant in a variant set (e.g., where support for a respective allele in each variant in the variant set is identified), thereby determining an observed frequency of the allele for the variant in the variant set. In some embodiments, each observed frequency corresponds to a respective allele frequency in the plurality of allele frequencies.

In some embodiments, a respective sequence read in the second plurality of sequence reads is deemed to support an allele of a first variant in the variant set when the respective sequence read contains the allele of the first variant. In some embodiments, a respective sequence read in the second plurality of sequence reads is deemed not to support the allele of the first variant in the variant set when the respective sequence read does not contain the first variant.

In some embodiments, a respective sequence read in the second plurality of sequence reads is deemed to support an allele of a first variant in the variant set when the respective sequence read contains the allele of the first variant, a respective sequence read in the second plurality of sequence reads is deemed not to support the allele of the first variant in the variant set when the respective sequence read maps on to the genomic region encompassing the allele but does not contain the allele of the first variant, and the observed frequency of the allele of the first variant is determined by a ratio or proportion between (i) a first number of unique cell-free nucleic acids, represented by the second plurality of sequence reads, that support the allele of the first variant and (ii) a second number of cell-free nucleic acids, represented by the second plurality of sequence reads, that map to the genomic region encompassing the allele irrespective of whether they support or do not support the allele of the first variant in the variant set, where the second number of cell-free nucleic acids includes the first number of cell-free nucleic acids.

In some embodiments, each respective variant in the variant set corresponds to a particular region in the reference genome of the subject. In some embodiments, a variant is an allele, including but not limited to point mutations and indels (e.g., insertions or deletions) within a gene.

In some embodiments, each allele in the plurality of alleles is a single nucleotide variant associated with a predetermined genomic location, an insertion mutation associated with a predetermined genomic location, a deletion mutation associated with a predetermined genomic location, a somatic copy number alteration, a nucleic acid rearrangement associated with a predetermined genomic locus, or an aberrant methylation pattern associated with a predetermined genomic location.

In some embodiments, the variant set comprises at least one variant, at least 10 variants, at least 20 variants, at least 30 variants, at least 40 variants, at least 50 variants, at least 60 variants, at least 70 variants, at least 80 variants, at least 90 variants, at least 100 variants, at least 200 variants, at least 300 variants, at least 400 variants, at least 500 variants, at least 600 variants, at least 700 variants, at least 800 variants, at least 900 variants, at least 1000 variants, at least 200 variants, at least 3000 variants, at least 400 variants, at least 5000 variants, at least 6000 variants, at least 7000 variants, at least 8000 variants, at least 9000 variants, at least 10,000 variants, at least 20,000 variants, at least 30,000 variants, at least 40,000 variants, at least 50,000 variants, at least 60,000 variants, at least 70,000 variants, at least 80,000 variants, at least 90,000 variants, or at least 100,00 variants. In some embodiments, the variant set comprises between 3000 and 4000 variants.

Referring to block 218, in some embodiments, the first biological sample and the second biological sample of the subject are one biological sample and the first plurality of cell-free nucleic acids is the same as the second plurality of cell-free nucleic acids. In some embodiments, the first biological sample and the second biological sample of the subject are one biological sample and this one biological sample is a plasma sample. In some embodiments, the first biological sample and the second biological sample of the subject are one biological sample and this one biological sample comprises blood, whole blood, plasma, serum, urine, cerebrospinal fluid, fecal, saliva, sweat, tears, pleural fluid, pericardial fluid, or peritoneal fluid of the subject. In some embodiments, the first biological sample and the second biological sample of the subject are one biological sample and this one biological sample consists of blood, whole blood, plasma, serum, urine, cerebrospinal fluid, fecal, saliva, sweat, tears, pleural fluid, pericardial fluid, or peritoneal fluid of the subject.

In some embodiments, the first and second biological samples are separate samples (e.g., taken on different days, taken from different liquid samples of the subject, etc.). In some embodiments, the first and/or second biological sample is plasma. In some embodiments, the first and/or second biological sample comprises blood, whole blood, plasma, serum, urine, cerebrospinal fluid, fecal, saliva, sweat, tears, pleural fluid, pericardial fluid, or peritoneal fluid of the subject. In some embodiments, the first and/or second biological sample consists of blood, whole blood, plasma, serum, urine, cerebrospinal fluid, fecal, saliva, sweat, tears, pleural fluid, pericardial fluid, or peritoneal fluid of the subject.

Referring to block 220, in some embodiments, in some embodiments, the first biological sample and the second biological sample of the subject are one (single) biological sample that is assayed by a targeted panel sequencing assay to provide both the plurality of bin values and the plurality of allele frequencies. In such embodiments, selected cell-free nucleic acids in the one biological sample are enriched using a plurality of probes before the targeted panel sequencing. Each probe in the plurality of probes includes a nucleic acid sequence that corresponds to one or more bins in the plurality of bins. In some embodiments, targeted panel sequencing is beneficial because it obtains significant information about regions of interest in the reference genome of the subject while being more efficient (e.g., with regard to use of materials for sequencing, length of time required for sequencing, etc.) than whole genome sequencing, for example. In other words, in some embodiments, targeted panel sequencing serves to obtain as much information as possible from the underlying data (e.g., at both the cell-free nucleic acid level and across genomic regions) while making the problem of determining tumor fraction (and/or tumor origin) for the subject computationally tractable. For example, a reference genome (e.g., a human reference genome) includes approximately 28 million CpG sites, while a targeted methylation panel directed to the reference genome includes fewer CpG sites (e.g., between 10,000 and 5 million CpG sites, between 100,000 and 3 million CpG sites, etc.

In some embodiments, the plurality of probes comprises at least 5,000, at least 10,000, at least 20,000, at least 30,000, at least 40,000, at least 50,000, at least 100,000, at least 200,000, at least 300,000, at least 400,000, at least 500,000, at least 600,000, at least 700,000, at least 800,000, at least 900,000, or at least 1,000,000 probes.

In some embodiments, the panel of genetic targets of the plurality of probes collectively covers 0.5 to 50 megabases of the reference genome. In some embodiments, the panel of genetic targets of the plurality of probes collectively covers 5 to 40 megabases of the reference genome, 10 to 30 megabases of the reference genome, 15 to 35 megabases of the reference genome, 20 to 30 megabases of the reference genome, 25 to 35 megabases of the reference genome, or 30 to 40 megabases of the reference genome.

In some embodiments, the first biological sample is assayed by whole genome sequencing to provide the plurality of bin values, and the second biological sample is assayed by a targeted panel sequencing to provide the plurality of allele frequencies, where selected cell-free nucleic acids in the second plurality of nucleic acids have been enriched using a plurality of probes before the targeted panel sequencing, and where each probe in the plurality of probes includes a nucleic acid sequence that maps to one or more bins in the plurality of bins.

In some embodiments, the whole genome sequencing comprises whole genome bisulfite sequencing. In such embodiments, there is overlap between genomic regions covered by the panel of genetic regions from targeted panel sequencing and the portions of the reference genome corresponding to bins in the plurality of bins.

In some embodiments, the first biological sample and the second biological sample are assayed by a targeted panel sequencing using a plurality of probes to provide, respectively, the plurality of bin values and the plurality of allele frequencies. In some embodiments, the first and second biological samples are assayed separately. In some embodiments, the first and second biological samples are assayed together (e.g., concurrently). In some embodiments, selected cell-free nucleic acids in the first biological sample and the second biological sample have been enriched using a plurality of probes (e.g., enrichment probes) before the targeted panel sequencing, and each probe in the plurality of probes includes a nucleic acid sequence that corresponds to one or more bins in the plurality of bins. In some embodiments, the targeted panel sequencing comprises bisulfite-based methylation sequencing. One or more sequencing methods for use in the assay embodiments provided here are described in more detail below.

Regardless of how the sequencing method used to analyzed the biological sample or samples, the same or similar methods can be used to derive copy number and allele frequency information.

In some embodiments, deriving the plurality of bin values further comprises using the first plurality of sequence reads to determine a respective number of unique cell-free nucleic acids represented by the plurality of sequence reads that map to each respective bin in the plurality of bins, thereby determining a corresponding bin count for each respective bin, and normalizing each respective bin count to obtain the plurality of bin values. In some embodiments, deriving the plurality of allele frequencies further comprises using the second plurality of sequence reads to identify support for an allele for a variant in a variant set, thereby determining an observed frequency of the allele for the variant in the variant set, where each observed frequency corresponds to a respective allele frequency in the plurality of allele frequencies.

In some embodiments, the first and/or second biological sample is processed to extract cell-free nucleic acids in preparation for sequencing analysis. By way of a non-limiting example, in some embodiments, cell-free nucleic acid is extracted from a blood sample collected from a subject in K2 EDTA tubes. Samples are processed within two hours of collection by double spinning of the blood first at ten minutes at 1000 g then plasma ten minutes at 2000 g. The plasma is then stored in 1 ml aliquots at −80° C. In this way, a suitable amount of plasma (e.g. 1-5 ml) is prepared from the first and/or second biological sample for the purposes of cell-free nucleic acid extraction. In some such embodiments cell-free nucleic acid is extracted using the QIAamp Circulating Nucleic Acid kit (Qiagen) and eluted into DNA Suspension Buffer (Sigma). In some embodiments, the purified cell-free nucleic acid is stored at −20° C. until use. See, for example, Swanton et al., 2017, “Phylogenetic ctDNA analysis depicts early stage lung cancer evolution,” Nature, 545(7655): 446-451, which is hereby incorporated herein by reference in its entirety. Other equivalent methods can be used to prepare cell-free nucleic acid using biological methods for the purpose of sequencing, and all such methods are within the scope of the present disclosure.

In some embodiments, the cell-free nucleic acid that is obtained from the first biological sample or the second biological sample is in any form of nucleic acid, or a combination thereof. For example, in some embodiments, the cell-free nucleic acid that is obtained from a biological sample is a mixture of RNA and DNA. In some embodiments, the cell-free nucleic acids of the first and second biological samples have undergone a conversion treatment comprising converting unmethylated cytosines or converting methylated cytosines.

The time between obtaining a biological sample and performing an assay, such as a sequence assay, can be optimized to improve the sensitivity and/or specificity of the assay or method. In some embodiments, a biological sample is obtained immediately before performing an assay. In some embodiments, a biological sample is obtained, and stored for a period of time (e.g., hours, days, or weeks) before performing an assay. In some embodiments, an assay can be performed on a sample within 1 day, 2 days, 3 days, 4 days, 5 days, 6 days, 1 week, 2 weeks, 3 weeks, 4 weeks, 5 weeks, 6 weeks, 7 weeks, 8 weeks, 3 months, 4 months, 5 months, 6 months, 1 year, or more than 1 year after obtaining the sample from the subject.

In some embodiments, the first plurality of sequence reads provides an average coverage of between 20× and 70,000× across the plurality of bins, and the second plurality of sequence reads provides an average coverage of between 1,000× and 70,000× across the plurality of alleles.

In some embodiments, the first plurality of sequence reads provides an average coverage of between 20× and 70,000× across the plurality of bins. In some embodiments, the first plurality of sequence reads provides an average coverage of between 20× and 1,000× across the plurality of bins. In some embodiments, the first plurality of sequence reads provides an average coverage of between 10× and 500×, between 20× and 1500×, or between between 20× and 3000× across the plurality of bins. In some embodiments, the first plurality of sequence reads provides an average coverage of between 1,000× and 70,000× across the plurality of bins. In some embodiments, the first plurality of sequence reads provides an average coverage of between 2,000× and 65,000×, between 5,000× and 60,000× or between 10,000× and 55,000× across the plurality of bins.

In some embodiments, the second plurality of sequence reads provides an average coverage of between 1,000× and 70,000× across the plurality of alleles. In some embodiments, the second plurality of sequence reads provides an average coverage of between 3,000× and 60,000×, between 5,000× and 50,000×, or between 7,500× and 45,000× across the plurality of alleles.

In some embodiments, for example when performing whole genome (bisulfite or non-bisulfite) sequencing, an average coverage rate of the first plurality of sequence reads and/or the second plurality of sequence reads that are taken from a biological sample (e.g., the first and/or second biological sample) is at least 1×, 2×, 3×, 4×, 5×, 6×, 7×, 8×, 9×, 10×, at least 20×, at least 30×, at least 40×, at least 50×, at least 100×, or at least 200× across the genome of the test subject.

In some embodiments, for example when sequencing (methylation- or non-methylation-based) using a targeted panel is performed, an average coverage rate of the first plurality of sequence reads and/or the second plurality of sequence reads that are taken from a biological sample (e.g., the first and/or second biological sample) of the subject is at least 100×, 200×, 500×, 1,000×, at least 2,000×, at least 3,000×, at least 4,000×, at least 5,000×, at least 10,000×, at least 15,000×, at least 20,000×, at least 25,000×, at least 30,000×, at least 40,000×, or at least 50,000× across selected regions in the genome of the subject. In some embodiments, the targeted panel of genes (e.g., and/or other selected regions in the genome of the subject) is within the range of 500±5 genes, within the range of 500±10 genes, within the range of 500±25 genes, within the range of 500±50 genes, within the range of 500±100 genes, within the range of 500±200 genes, within the range of 500±300 genes, or within the range of 500±400 genes. In some embodiments, the targeted panel of genes (e.g., and/or other selected regions in the genome of the subject) is within the range of 50±5 genes, within in the range of 50±10 genes, within the range of 50±15 genes, within the range of 50±20 genes, within the range of 50±25 genes, within the range of 50±30 genes, within the range of 50±35 genes, within the range of 50±40 genes, of within the range of 50±45 genes. In some such embodiments, the targeted assay looks for single nucleotide variants in the targeted panel of genes (e.g., and/or other selected regions in the genome of the subject), insertions in the targeted panel of genes, deletions in the targeted panel of genes, somatic copy number alterations (SCNAs) in the targeted panel of genes, or re-arrangements affecting the targeted panel of genes. In some embodiments, SCNAs can be detected from either WGBS or WGS data. In some embodiments, the test subject is human and the first feature is a single nucleotide variant count, an insertion mutation count, a deletion mutation count, or a nucleic acid rearrangement count across the human reference genome.

In some embodiments, the plurality of probes comprises 1,000 to 2,000,000 probes, where each probe is designed to bind and enrich cell-free nucleic acids in the first and/or second biological sample that contain at least one predetermined epigenetic feature such as a CpG site. In some embodiments, the plurality of probes comprises 1,500,000 probes or fewer, 1,400,000 probes or fewer, 1,300,000 probes or fewer, 1,200,000 probes or fewer, 1,100,000 probes or fewer, 1,000,000 probes or fewer, 900,000 probes or fewer, 800,000 probes or fewer, 700,000 probes or fewer, 600,000 probes or fewer, 500,000 probes or fewer, 400,000 probes or fewer, 300,000 probes or fewer, 200,000 probes or fewer, 100,000 probes or fewer, 90,000 probes or fewer, 80,000 probes or fewer, 70,000 probes or fewer, 60,000 probes or fewer, 50,000 probes or fewer, 40,000 probes or fewer, 30,000 probes or fewer, 20,000 probes or fewer, 10,000 probes or fewer, 9,000 probes or fewer, 8,000 probes or fewer, 7,000 probes or fewer, 6,000 probes or fewer, 5,000 probes or fewer, 4,000 probes or fewer, 3,000 probes or fewer, 2,000 probes or fewer, or 1,000 probes or fewer.

A whole genome sequencing assay refers to a physical assay that generates sequence reads for a whole genome or a substantial portion of the whole genome that can be used to determine large variations such as copy number variations or copy number aberrations. Such a physical assay may employ whole genome sequencing techniques or whole exome sequencing techniques.

In some of such embodiments, the whole genome bisulfite sequencing identifies one or more methylation state vectors as described, for example, in U.S. patent application Ser. No. 16/352,602, entitled “Methylation Fragment Anomaly Detection,” filed Mar. 13, 2019, which is hereby incorporated by reference herein in its entirety.

The sequencing assay (e.g., first nucleic acid sequencing, second nucleic acid sequencing) can comprise any form of sequencing that can be used to obtain a number of sequence reads measured from cell-free nucleic acids, including, but not limited to, high-throughput sequencing systems such as the Roche 454 platform, the Applied Biosystems SOLID platform, the Helicos True Single Molecule DNA sequencing technology, the sequencing-by-hybridization platform from Affymetrix Inc., the single molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used to obtain sequence reads 140 from the cell-free nucleic acid obtained from the first and/or second biological sample.

In some embodiments, the first nucleic acid sequencing and/or second nucleic acid sequencing is sequencing-by-synthesis and reversible terminator-based sequencing (e.g., Illumina's Genome Analyzer; Genome Analyzer II; HISEQ 2000; HISEQ 2500 (Illumina, San Diego Calif.)). In some such embodiments, millions of cell-free nucleic acid (e.g., DNA) fragments are sequenced in parallel. In one example of this type of sequencing technology, a flow cell is used that contains an optically transparent slide with eight or more individual lanes on the surfaces of which are bound oligonucleotide anchors (e.g., adaptor primers). A flow cell often is a solid support that is configured to retain and/or allow the orderly passage of reagent solutions over bound analytes. In some instances, flow cells are planar in shape, optically transparent, generally in the millimeter or sub-millimeter scale, and often have channels or lanes in which the analyte/reagent interaction occurs. In some embodiments, a cell-free nucleic acid sample includes a signal or tag that facilitates detection. In some such embodiments, the acquisition of sequence reads from the cell-free nucleic acid obtained from the first and/or second biological sample includes obtaining quantification information of the signal or tag via a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

Referring to block 222, in some embodiments, a respective probe in the plurality of probes includes a respective nucleic acid sequence that varies with respect to the reference genomic sequence, or a portion thereof, as represented by a bin in the plurality of bins by one or more transitions. Each respective transition in the one or more transitions occurs at a respective un-methylated CpG dinucleotide site in the respective genomic region.

Referring to block 224, in some embodiments, a respective probe in the plurality of probes includes a respective nucleic acid sequence that varies with respect to the reference genomic sequence, or a portion thereof, as represented by a bin in the plurality of bins by one or more transitions. Each respective transition in the one or more transitions occurs at a respective methylated CpG dinucleotide site in the respective genomic region.

In some embodiments, a probe in the plurality of probes enriches selected cell-free nucleic acids in the first and/or second biological sample that contain 50 or fewer predetermined CpG sites, 40 or fewer predetermined CpG sites, 30 or fewer predetermined CpG sites, 25 or fewer predetermined CpG sites, 22 or fewer predetermined CpG sites, 20 or fewer predetermined CpG sites, 18 or fewer predetermined CpG sites, 15 or fewer predetermined CpG sites, 12 or fewer predetermined CpG sites, 10 or fewer predetermined CpG sites, 5 or fewer predetermined CpG sites, 3 or fewer predetermined CpG sites. In some embodiments, a probe in the plurality of probes is about 20 bp, about 25 bp, about 30 bp, about 35 bp, about 40 bp, about 45 bp, or about 50 bp in length.

In some embodiments, the method further comprises subjecting the first and/or second plurality of cell-free nucleic acids to a conversion treatment, prior to assaying the first and/or the second biological sample (e.g., by whole genome or targeted panel sequencing).

In some embodiments, the method further comprises subjecting the first and/or second plurality of cell-free nucleic acids to a bisulfite conversion treatment, prior to assaying the first and/or the second biological sample (e.g., by whole genome or targeted panel sequencing). In some embodiments, the bisulfite conversion treatment causes one or more unmethylated cytosines in the plurality of cell-free nucleic acids to be converted to one or more corresponding uracils, and the targeted panel sequencing of the plurality of cell-free nucleic acids reads out the one or more corresponding uracils as one or more corresponding thymines.

In some embodiments, the method further comprises subjecting the first and/or second plurality of cell-free nucleic acids to one or more enzymatic conversion treatments, prior to assaying the first and/or the second biological sample (e.g., by whole genome or targeted panel sequencing). In some embodiments, the one or more enzymatic conversion treatments cause one or more methylated cytosines in the plurality of cell-free nucleic acids to be converted to one or more corresponding uracils. In such embodiments, targeted panel sequencing of the first and/or second plurality of cell-free nucleic acids reads out the one or more corresponding uracils as one or more corresponding thymines.

In some embodiments, a probe in the plurality of probes includes a respective nucleic acid sequence that is complementary or substantially complementary to the reference genome or a portion thereof that includes the first cytosine and the second cytosine. In some embodiments, the probe includes a first guanosine for the first cytosine, and with the exception that the probe further includes an adenine for the second cytosine. In some embodiments, the bisulfite conversion treatment causes the targeted sequencing to selectively amplify cell-free nucleic acid sequences that originate from the cancer of origin over the absence of the cancer of origin. In some embodiments, the enrichment probes are designed to be complementary to the converted sequences. In some embodiments, the enrichment probes are only partially complementary to the reference genome. For example, DNA molecule (1) includes three CpG sites, only one of which is methylated where non-CpG related nucleotides are marked as “X”:

XCmGXXCGXXXXXXXXXXCG (1)

After bisulfite treatment, as described above, the sequence is converted to:

XCGXXUGXXXXXXXXXXUG (2)

After PCR amplification and sequencing reactions, the sequence is read out as:

XCGXXTGXXXXXXXXXXTG. (3)

In this example, only the methylated C is subsequently read as C; the other Cs (e.g., those that were un-methylated) are eventually read as T post-conversion treatment after being first converted to Uracil (U) first. In some embodiments, an enrichment probe (e.g., a probe in the first plurality of probes) will have a sequence that is complementary to sequence (2) not sequence (1).

In some embodiments, methylation patterns identified from sequencing analysis of a biological sample of the subject can also be used to determine a cancer condition of the subject.

For example, U.S. Patent Application No. 62/983,443, entitled “Identifying Methylation Patterns that Discriminate or Indicate a Cancer Condition,” filed on Feb. 28, 2020, which is hereby incorporated by reference in its entirety, discloses multiple methods of identifying methylation patterns that discriminate specific cancer conditions of the subject. Specifically, in some embodiments, each cancer condition (e.g., cancer of origin) in the group of cancer conditions corresponds to a respective pattern of abnormal methylation (e.g., a qualifying methylation pattern) across a reference genome or across a subset of the reference genome (e.g., as evaluated by targeted panel sequencing). To determine the cancer condition of a particular subject, the method evaluates a plurality of genomic regions of interest, and generates, for each genomic region in the plurality of genomic regions, a corresponding count of fragments with methylation patterns that map to the respective genomic region (e.g., there is a respective count of fragments for each possible methylation pattern identified in fragments mapping to the respective genomic region). The method then compares the fragment counts across the plurality of genomic regions for the subject to a database (e.g., library) of methylation patterns corresponding to different cancer conditions (e.g., where each cancer condition has corresponding fragment counts for a respective subset of genomic regions within the plurality of genomic regions) to determine a probable cancer condition for the subject, where the cancer condition corresponds to cancer vs. non-cancer, type of cancer, and/or tissue-of-origin. In some embodiments, the method is used to identify a cancer condition of the subject for input into downstream applications (e.g., for estimating tumor fraction and/or determining minimal residual disease of the subject). In some embodiments, the plurality of bins used in the present disclosure are selected to represent portions of the genome identified in U.S. Patent Application No. 62/983,443 that contain the methylation patterns associated with any single or any combination of cancers evaluated in U.S. Patent Application No. 62/983,443. In some embodiments, the plurality of alleles used in the present disclosure are selected from the epigenetic features (e.g., methylation patterns) identified in U.S. Patent Application No. 62/983,443 that are associated with any single or any combination of cancers evaluated in U.S. Patent Application No. 62/983,443.

As another example, U.S. patent application Ser. No. 15/931,022, entitled “Model-Based Featurization and Classification,” filed on May 13, 2020, which is hereby incorporated by reference in its entirety, discloses the development of probabilistic models using methylation states of genomic regions (e.g., determined from fragments as represented by sequence reads that map to the genomic regions) to identify methylation features that correspond to distinct cancer conditions. In some embodiments, the plurality of bins used in the present disclosure are selected to represent portions of the genome identified in U.S. patent application Ser. No. 15/931,022 that contain the methylation patterns associated with any single or any combination of cancers evaluated in U.S. patent application Ser. No. 15/931,022. In some embodiments, the plurality of alleles used in the present disclosure are selected from the epigenetic features (e.g., methylation patterns) identified in U.S. patent application Ser. No. 15/931,022 that are associated with any single or any combination of cancers evaluated in U.S. patent application Ser. No. 15/931,022.

In some embodiments, a first cancer condition is characterized by a first epigenetic cytosine methylation pattern. In some embodiments, a first cytosine methylation pattern at a first genomic locus of the species is characteristic of the first disease condition, and a second cytosine methylation pattern, different from the first cytosine methylation pattern, at the first genomic locus is characteristic of an absence of the first disease condition. In some embodiments, the method further comprises subjecting the plurality of nucleic acids to an enzymatic treatment, prior to assaying the first and/or the second biological sample (e.g., by whole genome or targeted panel sequencing). In some embodiments, the enzymatic treatment causes a plurality of unmethylated cytosines in the plurality of nucleic acids to be converted to a plurality of corresponding modified bases. In some embodiments, a first probe in the plurality of probes includes a respective nucleic acid sequence that is complementary or substantially complementary to the first genomic locus, with the exception that the first probe is only complementary to the first genomic locus upon conversion of methylated cytosines of the first methylation pattern by the epigenetic enzymatic treatment, thereby causing the targeted sequencing to selectively read, through the first probe, for the cancer condition over the absence of the cancer condition.

In an alternate embodiment, methylated cytosines instead of unmethylated cytosines are converted. In the human genome, 95% of the cytosines are not methylated, which means bisulfite conversion following standard practices will result in DNA fragments that contain many nucleic acid base uracils that will be read out as thymines (e.g., the final sequence reads are heavily populated with thymines). Such a preponderance of thymines results in an unbalanced genome, which has the potential to introduce complications in mapping sequence reads and other downstream methods. To resolve this, enzymatic conversion processes are used in some embodiments to treat the nucleic acid prior to sequencing. For example, Liu et al. developed TAPS (TET-Assisted Pyridine borane Sequencing), a method that combines pyridine borane reactions with the reaction of TET, a human enzyme. See, Liu et al., 2019, “Bisulfite-free direct detection of 5-methylcytosine and 5-hydroxymethylcytosine at base resolution,” Nature Biotechnol 37, pp. 424-429, which is hereby incorporated by reference. Based on their methods, only the methylated Cs will be converted. There are variations of the method described by Liu et al. for detecting methyl versus hydroxy methyl modification.

In some embodiments, the plurality of corresponding modified bases is a plurality of uracils. In some embodiments, the enzymatic treatment comprises: i) exposing the plurality of cell-free nucleic acids to a ten-eleven translocation (TET) dioxygenase, and ii) exposing the cell-free plurality of nucleic acids to a borane based reducing agent after exposure to the TET dioxygenase (e.g., as described by Liu et at.; see FIG. 1D, left hand path). In some embodiments, the method further comprises exposing the plurality of nucleic acids to β-glucosyltransferase prior to the exposing (i) (e.g., as described by Liu et at.; see FIG. 1D, middle path). In some embodiments, the method further comprises exposing the plurality of nucleic acids to KRuO₄ prior to the exposing (i) (e.g., as described by Liu et at; see FIG. 1D, right hand path). In some embodiments, the borane based reducing comprises pyridine borane or 2-picoline borane.

Referring to block 226, in some embodiments, the respective corresponding region of the reference genome, or a portion thereof, for each corresponding bin in a first set of bins in the plurality of bins is complementary or substantially complementary to the sequences of two or more probes in a plurality of probes used in a targeted nucleic acid sequencing to generate the plurality of bin values (e.g., on-target regions). In some embodiments, the respective corresponding region of the reference genome, or a portion thereof, for each corresponding bin in a second set of bins in the plurality of bins is not represented by a sequence of any probe in the plurality of probes (e.g., off-target regions).

In some embodiments, a portion of the reference genome corresponding to a bin in the second set of bins comprises a sequence of contiguous nucleic acid bases. In some embodiments, each portion of the reference genome has the same size. In some embodiments, one or more of the corresponding portions of the reference genome are different sizes. In some embodiments, each portion of a reference genome corresponding to a bin the second set of bins comprises at least 10 contiguous bases, at least 15 contiguous bases, at least 20 contiguous bases, at least 30 contiguous bases, at least 40 contiguous bases, at least 50 contiguous bases, at least 60 contiguous bases, at least 70 contiguous bases, at least 80 contiguous bases, at least 90 contiguous bases, at least 100 contiguous bases, at least 150 contiguous bases, at least 200 contiguous bases, at least 250 contiguous bases, at least 300 contiguous bases, at least 400 contiguous bases, or at least 500 contiguous bases.

Referring to block 228, in some embodiments, a respective probe in the plurality of probes includes a corresponding nucleic acid sequence that is complementary or substantially complementary to the respective genomic region.

Referring to block 230, in some embodiments, a respective probe in the plurality of probes includes a corresponding nucleic acid sequence that is complementary or substantially complementary to the reference genome, or a portion thereof, as represented by a bin in the plurality of bins with the exception of one or more transitions. In some embodiments, each respective transition in the one or more transitions occurs at a respective un-methylated CpG dinucleotide site in the reference genome.

Referring to block 232, in some embodiments, a respective probe in the plurality of probes includes a corresponding nucleic acid sequence that is complementary or substantially complementary to the reference genome, or a portion thereof, as represented by a bin in the plurality of bins with the exception of one or more transitions. In some embodiments, each respective transition in the one or more transitions occurs at a respective methylated CpG dinucleotide site in the reference genome.

Referring to block 234, in some embodiments, each probe in the plurality of probes includes a respective nucleic acid sequence that is complementary or substantially complementary to the reference genome, or a portion thereof, as represented by a bin in the plurality of bins, with the exception that the probe includes an adenine to complement a thymine corresponding to a methylated or unmethylated cytosine in a selected cell-free nucleic acid (e.g., an original cell-free nucleic acid fragment).

In a reference genome, a significant percentage of CpG sites are typically unmethylated (e.g., 95-97% of possible sites). See e.g., Pfeifer 2018 Int J Mol Sci 19, 1166. As discussed above, in some embodiments, either methylated or unmethylated cytosines from CpG sites are converted (e.g., via a conversion treatment) to uracils in one or more target cell-free nucleic acid fragments (e.g., original cell-free nucleic acids). In such embodiments, after two or more rounds of PCR (e.g., performed as part of the sequencing analysis process), in the resulting sequence reads each such uracil from the original cell-free nucleic acid will be read as a thymine. In such embodiments, one or more probes in the plurality of probes will include an adenine as a complement to the resulting thymines.

Referring to block 236, in some embodiments, the method further comprises subjecting the cell-free nucleic acids of the first and second biological samples to a conversion treatment, prior to the obtaining a), that causes i) one or more unmethylated cytosines in the first or second plurality of cell-free nucleic acids to be converted one or more corresponding bases or ii) one or more methylated cytosines in the first or second plurality of cell-free nucleic acids to be converted to one or more corresponding bases.

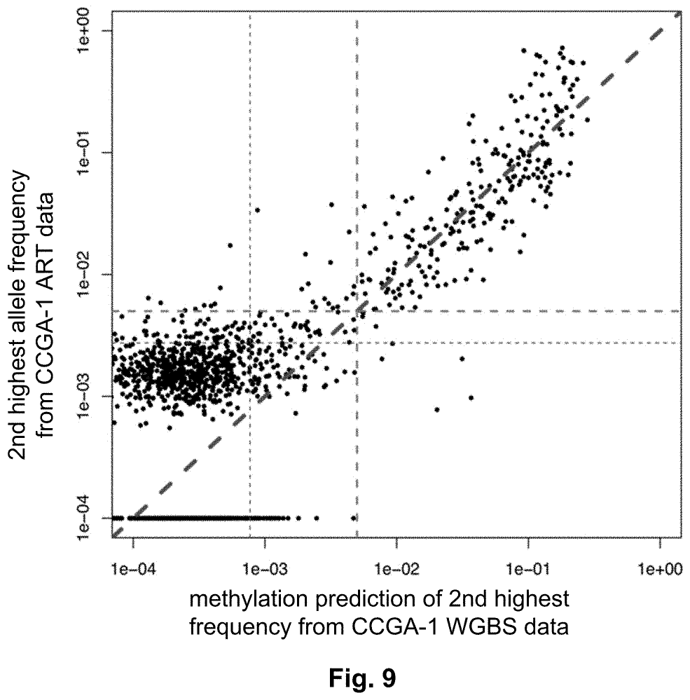

As described in Example 1, both separately and together, copy number and allele frequency of a respective subject are correlated with the known tumor fraction of a subject. Similarly, for example as shown in FIG. 9, allele frequency itself can be predicted using methylation data from whole genome bisulfite (or other methylation) sequencing. This correlation between allele frequency and methylation data—in combination with the rest of the methods disclosed herein—suggests that methylation data can also be used to predict tumor fraction, either alone or in combination with copy number.

Referring to block 238, in some embodiments, the plurality of allele frequencies are derived by using the second plurality of sequence reads to identify support for an allele for a variant in a variant set, thereby determining an observed frequency of the allele for the variant in the variant set. Each observed frequency corresponds to a respective allele frequency in the plurality of allele frequencies.

Referring to block 240, in some embodiments, a respective sequence read in the second plurality of sequence reads is deemed to support an allele of a first variant in the variant set when the respective sequence read contains the allele of the first variant. A respective sequence read in the second plurality of sequence reads is deemed not to support an allele of a first variant in the variant set when the respective sequence read does not contain the allele of the first variant. The observed frequency of the allele of the first variant is determined by a ratio or proportion between (i) a first number of unique cell-free nucleic acids, represented by the second plurality of sequence reads, that support the allele of the first variant and (ii) a second number of cell-free nucleic acids, represented by the second plurality of sequence reads, that map to the genomic region encompassing the allele irrespective of whether they support or do not support the allele of the first variant in the variant set, where the second number of cell-free nucleic acids includes the first number of cell-free nucleic acids.

Referring to block 242, in some embodiments, each respective variant in the variant set corresponds to a particular region in the reference genome of the subject. In other words, each variant is associate with a particular, unique, portion (locus) of the reference genome.

Block 250. Referring to block 250 of FIG. 2D, the method continues by applying, to a reference model, at least the plurality of copy number values and the plurality of allele frequencies, or a plurality of features derived therefrom, thereby determining the tumor fraction of the subject. In some embodiments, tumor fraction estimates are calculated based on the assumption that one or more methylation state patterns in a biological sample of the subject (e.g., cfDNA and/or plasma) are tumor-derived, and that the frequency of such tumor-derived variant alleles are directly proportional to the fraction of cancer cells to normal cells (e.g., the tumor fraction).

In some embodiments, tumor fraction estimation uses likelihoods that the copy number values in the plurality of copy number values (e.g., corresponding to various bins that may include epigenetic variations) are associated with cancer (e.g., are determined from cancer-derived fragments). In some embodiments, tumor fraction estimation uses likelihoods that allele frequencies in the plurality of allele frequencies are associated with cancer (e.g., are determined based on cancer-derived fragments). There are various methods of determining such likelihoods, some of which are described in U.S. patent application Ser. No. 16/719,902, entitled “Systems and Methods for Estimating Cell Source Fractions using Methylation Information,” filed Dec. 18, 2019 and U.S. patent application Ser. No. 16/850,634 entitled “Systems and Methods for Tumor Fraction Estimation from Small Variants,” filed Apr. 16, 2020, both of which are hereby incorporated by reference in their entireties.

In some embodiments, the tumor fraction of the subject is in the range of 0.001 and 1.0. In some embodiments, the tumor fraction of the subject is at least 0.001, at least 0.005, at least 0.01, at least 0.05, at least 0.1, at least 0.2, at least 0.3, at least 0.4, at least 0.5, at least 0.6, at least 0.7, at least 0.8, at least 0.9, or at least 1.0.

In some embodiments, determining the tumor fraction of the subject further identifies a cancer of origin of the subject. In other words, application of the plurality of copy number values and the plurality of allele frequencies, or a plurality of features derived therefrom to the reference model causes the reference model to further indicate the caner of origin of the subject. In some embodiments, the cancer of origin comprises a first cancer condition selected from the group consisting of non-cancer, breast cancer, lung cancer, prostate cancer, colorectal cancer, renal cancer, uterine cancer, pancreatic cancer, cancer of the esophagus, lymphoma, head/neck cancer, ovarian cancer, hepatobiliary cancer, melanoma, cervical cancer, multiple myeloma, leukemia, thyroid cancer, bladder cancer, gastric cancer, nasopharyngeal cancer, liver cancer, or a combination thereof.

In some embodiments, the cancer of origin comprises at least a first cancer condition and a second cancer condition each selected from the group consisting of breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, nasopharyngeal cancer, liver cancer, or a combination thereof.

In some embodiments, the first and/or second cancer condition comprises a stage of a breast cancer, a stage of a lung cancer, a stage of a prostate cancer, a stage of a colorectal cancer, a stage of a renal cancer, a stage of a uterine cancer, a stage of a pancreatic cancer, a stage of a cancer of the esophagus, a stage of a lymphoma, a stage of a head/neck cancer, a stage of a ovarian cancer, a stage of a hepatobiliary cancer, a stage of a melanoma, a stage of a cervical cancer, a stage of a multiple myeloma, a stage of a leukemia, a stage of a thyroid cancer, a stage of a bladder cancer, a stage of a gastric cancer, a stage of nasopharyngeal cancer, a stage of liver cancer, or a combination thereof.

In some embodiments, determining the tumor fraction of the subject further includes providing a treatment recommendation (e.g., a cancer treatment) to the subject, where the treatment recommendation is based at least in part on the tumor fraction (e.g., how progressed the disease is) and the cancer of origin.

In some embodiments, the method further comprises determining the tumor fraction of the subject at one or more time points (e.g., before or after treatment) to monitor disease progression or to monitor treatment effectiveness (e.g., therapeutic efficacy). An increase in tumor fraction over time (e.g., at a second, later time point) can indicate disease progression, and conversely a decrease in the tumor fraction over time (e.g., at a second, later time point) can indicate successful treatment.

In some embodiments, the method is repeated at each respective time point in a plurality of time points (e.g., two or more time points, three or more time points four or more time points) across an epoch, thereby obtaining a corresponding tumor fraction, in a plurality of tumor fractions, for the subject at each respective time point and using the plurality of tumor fractions to determine a state or progression of a disease condition in the subject during the epoch in the form of an increase or decrease of the first tumor fraction over the epoch. In some such embodiments, the epoch is a period of months (e.g., between two and ten months, etc.) and each time point in the plurality of time points is a different time point in the period of months. In some embodiments, the epoch is a period of years (e.g., between two and ten years) and each time point in the plurality of time points is a different time point in the period of years. In some embodiments, the epoch is a period of hours (e.g., between one hour and six hours) and each time point in the plurality of time points is a different time point in the period of hours.

In some embodiments, the method further comprises changing a diagnosis of the subject when the first tumor fraction of the subject is observed to change by a threshold amount across the epoch. In some embodiments, the method further comprises changing a prognosis of the subject when the first tumor fraction of the subject is observed to change by a threshold amount across the epoch. In some embodiments, the method further comprises changing a treatment of the subject when the first tumor fraction of the subject is observed to change by a threshold amount across the epoch. In some of the forgoing embodiments, the threshold is greater than ten percent, greater than twenty percent, greater than thirty percent, greater than forty percent, greater than fifty percent, greater than two-fold, greater than three-fold, or greater than five-fold.

In certain embodiments, the method is conducted at a first time point that is before a cancer treatment (e.g., before a resection surgery or a therapeutic intervention) as well as at a second time point that is after a cancer treatment (e.g., after a resection surgery or therapeutic intervention), and the disclosed methods are used to monitor the effectiveness of the treatment by comparison of the tumor fraction determined by the disclosed methods at each time point. For example, if the tumor fraction at the second time point decreases compared to the tumor fraction at the first time point, then the treatment is deemed successful. However, if the tumor fraction at the second time point increases compared to the tumor fraction at the first time point, then the treatment is deemed not successful. In other embodiments, both the first and second time points are before a cancer treatment (e.g., before a resection surgery or a therapeutic intervention). In still other embodiments, both the first and the second time points are after a cancer treatment (e.g., before a resection surgery or a therapeutic intervention) and the method is used to monitor the effectiveness of the treatment or loss of effectiveness of the treatment. In still other embodiments, biological samples (cfDNA samples) may be obtained from a cancer patient at a first and second time point and analyzed, e.g., to monitor cancer progression, to determine if a cancer is in remission (e.g., after treatment), to monitor or detect residual disease or recurrence of disease, or to monitor treatment (e.g., therapeutic) efficacy.

Those of skill in the art will readily appreciate that biological samples can be obtained from a cancer patient over any number of time points and analyzed in accordance with the methods of the disclosure to monitor a cancer state (e.g., via tumor fraction) in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 30 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, biological samples can be obtained from the patient at least once every 3 months, at least once every 6 months, at least once a year, at least once every 2 years, at least once every 3 years, at least once every 4 years, or at least once every 5 years.

In some embodiments, the reference model is a multivariate logistic regression, a neural network, a convolutional neural network, a support vector machine (SVM), a decision tree, a regression algorithm, or a supervised clustering model.

Logistic regression algorithms, including multivariate logistic regression, are disclosed in Agresti, An Introduction to Categorical Data Analysis, 1996, Chapter 5, pp. 103-144, John Wiley & Son, New York, which is hereby incorporated by reference.

Neural network algorithms, including convolutional neural network algorithms, are disclosed in See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference.

SVM algorithms are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data training set (e.g., by tumor fraction value) with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space.

Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

Clustering is described at pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined.

Similarity measures are discussed in Section 6.7 of Duda 1973, where it is stated that one way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 218 of Duda 1973.

Once a method for measuring “similarity” or “dissimilarity” between points in a dataset has been selected, clustering requires a criterion function that measures the clustering quality of any partition of the data. Partitions of the dataset that extremize the criterion function are used to cluster the data. See page 217 of Duda 1973. Criterion functions are discussed in Section 6.8 of Duda 1973.

More recently, Duda et al., Pattern Classification, 2^{nd} edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J., each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. Such clustering can be on the set of first features {p₁, . . . , p_{N-K}} (or the principal components derived from the set of first features). In some embodiments, the clustering comprises unsupervised clustering where no preconceived notion of what clusters should form when the training set is clustered are imposed.

In some embodiments, the tumor fraction of the subject or other information provided by the reference model is used to determine and apply a treatment regimen to the test subject (e.g., based at least in part on the output of the reference model upon application, to the reference model, at least the plurality of copy number values and the plurality of allele frequencies, or a plurality of features derived therefrom. In some embodiments, the treatment regimen comprises applying an agent for cancer to the test subject based on the tumor fraction determined by the reference model for the test subject. Non-limiting examples of agents for cancer that can be applied based on an output of the reference model include, but are not limited to, hormones, immune therapies, radiography, and cancer drugs. Examples of cancer drugs include, but are not limited to, Lenalidomid, Pembrolizumab, Trastuzumab, Bevacizumab, Rituximab, Ibrutinib, Human Papillomavirus Quadrivalent (Types 6, 11, 16, and 18) Vaccine, Pertuzumab, Pemetrexed, Nilotinib, Nilotinib, Denosumab, Abiraterone acetate, Promacta, Imatinib, Everolimus, Palbociclib, Erlotinib, Bortezomib, and Bortezomib.

Deriving Features from Copy Number Values and/or Allele Frequencies

As described in relation to block 250 of FIG. 2D, either the copy number values and the allele frequencies, or a plurality of features derived from one or both of the copy number values and allele frequencies, are applied to the reference model to determine the tumor fraction of the subject. A feature (also referred to herein as a feature value) can be the computational result of inputting the copy number counts (e.g., as determined from the bin values) and/or the allele frequencies into one or more dimensionality reduction (feature extraction) functions or algorithms.

In some embodiments, the feature values collectively determine a vector for the subject. For example, in embodiments in which each feature extraction function from the one or more feature extraction functions is a principal component, each feature value includes the copy number counts or the allele frequencies projected onto a particular principal component.

Feature extraction functions can be derived using any suitable method. In some embodiments, they are derived through the training of a reference model (e.g., using a plurality of subjects for reference). For example, in some embodiments, a suitable feature extraction function comprises applying a dimension reduction algorithm to the subjects in the plurality of subjects that have a range of tumor fractions, thereby identifying the corresponding subset of the feature extraction functions (e.g., principal components) to use for determining tumor fraction of a test subject.

The dimension reduction algorithm can be a linear dimension reduction algorithm or a non-linear dimension reduction algorithm. In some embodiments, the dimension reduction algorithm is principal component analysis algorithm, a factor analysis algorithm, Sammon mapping, curvilinear components analysis, a stochastic neighbor embedding (SNE) algorithm, an Isomap algorithm, a maximum variance unfolding algorithm, a locally linear embedding algorithm, a t-SNE algorithm, a non-negative matrix factorization algorithm, a kernel principal component analysis algorithm, a graph-based kernel principal component analysis algorithm, a linear discriminant analysis algorithm, a generalized discriminant analysis algorithm, a uniform manifold approximation and projection (UMAP) algorithm, a LargeVis algorithm, a Laplacian Eigenmap algorithm, or a Fisher's linear discriminant analysis algorithm. See, for example, Fodor, 2002, “A survey of dimension reduction techniques,” Center for Applied Scientific Computing, Lawrence Livermore National, Technical Report UCRL-ID-148494; Cunningham, 2007, “Dimension Reduction,” University College Dublin, Technical Report UCD-CSI-2007-7, Zahorian et al., 2011, “Nonlinear Dimensionality Reduction Methods for Use with Automatic Speech Recognition,” Speech Technologies. doi:10.5772/16863. ISBN 978-953-307-996-7; and Lakshmi et al. (18 Aug. 2016). 2016 IEEE 6th International Conference on Advanced Computing (IACC). pp. 31-34. doi:10.1109/IACC.2016.16, ISBN 978-1-4673-8286-1, each of which is hereby incorporated by reference.

In some embodiments, the dimensionality reduction algorithm is a regression algorithm (e.g., for the dimensionality reduction and/or training the reference model to determine tumor fraction). The regression algorithm can be any type of regression. In some embodiments, the regression algorithm is linear regression or random forest regression. For example, in some embodiments, the regression algorithm is logistic regression. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the logistic regression is logistic least absolute shrinkage and selection operator (LASSO) regression. Example logistic regression algorithms are disclosed in Agresti, An Introduction to Categorical Data Analysis, 1996, Chapter 5, pp. 103-144, John Wiley & Son, New York, which is hereby incorporated by reference. In some embodiments, the regression algorithm is linear regression with L1 or L2 regularization.

Training a Reference Model to Determine a Tumor Fraction

As part of determining a tumor fraction for a subject of a species, as described above with regard to blocks 202-250, the reference model is trained against a plurality of reference subjects prior to application to a test subject. Such a reference model uses information from a plurality of reference subjects with known genotypic information and cancer conditions (e.g., from the whole genome sequencing or targeted panel sequencing within the CCGA studies, discussed below). In some embodiments, genotypic information for each reference subject is generated from a TCGA dataset, as described below.

The present disclosure further provides methods for training a reference model to determine a tumor fraction of a test subject. A training dataset is obtained, in electronic form, that comprises, for each respective reference subject in a plurality of reference subjects: (i) a corresponding plurality of bin values, each respective bin value in the corresponding plurality of bin values being for a corresponding bin in a plurality of bins, (ii) a corresponding plurality of allele frequencies for a corresponding plurality of alleles, and (iii) a corresponding tumor fraction value for the respective reference subject. Each respective bin in the plurality of bins represents a corresponding region of a reference genome of the plurality of reference subjects. As described above, with reference to block 210, in some embodiments, each bin is a specified size. In some embodiments, each respective bin in the plurality of bins represents a non-overlapping corresponding region of the reference genome of the plurality of reference subjects.

Each corresponding plurality of bin values is derived from alignment of a corresponding first plurality of sequence reads, determined by a corresponding first nucleic acid sequencing of a corresponding first plurality of cell-free nucleic acids in a corresponding first biological sample, to a reference genome of the species. In some embodiments, the first biological sample comprises a liquid sample of a respective reference subject in the plurality of reference subjects. In some embodiments, the corresponding first plurality of cell-free nucleic acids comprises at least 1000 cell-free nucleic acids.

Each corresponding plurality of allele frequencies is derived from alignment of a corresponding second plurality of sequence reads, determined by a corresponding second nucleic acid sequencing of a corresponding second plurality of cell-free nucleic acids in a second biological sample, to the reference genome. In some embodiments, the corresponding second biological sample comprises a liquid sample of a respective reference subject in the plurality of reference subjects. In some embodiments, the corresponding second plurality of cell-free nucleic acids comprises at least 1000 cell-free nucleic acids.

The method continues by determining, for each respective reference subject in the plurality of subjects, a respective plurality of copy number values from the corresponding plurality of bins values for the respective reference subject (e.g., as described above with reference to blocks 212-214).

After collecting the above mentioned information, the method obtains the reference model using at least (i) the respective plurality of copy number values, (ii) the respective plurality of allele frequencies, or a respective plurality of features derived from (i) and (ii), and (iii) the tumor fraction value of each respective reference subject in the plurality of reference subjects.

In some embodiments, each respective plurality of features derived from the respective plurality of copy number values and/or the respective plurality of allele frequencies is extracted as described with regard to block 250 above.

In some embodiments, the first biological sample of each respective reference subject is assayed by a targeted panel sequencing with a plurality of probes targeting a panel of genetic regions to provide the plurality of bin values. In some embodiments, a plurality of cell-free nucleic acids are obtained from the first biological sample and subjected to targeted panel sequencing (for example as described above with regards to block 220).

In some embodiments, the plurality of reference subjects comprises at least 10 subjects, at least 20 subjects, at least 30 subjects, at least 40 subjects, at least 50 subjects, at least 60 subjects, at least 70 subjects, at least 80 subjects, at least 90 subjects, at least 100 subjects. At least 150 subjects, at least 250 subjects, at least 500 subjects, at least 750 subjects, at least 1000 subjects, or at least 1500 subjects. In some embodiments, each reference subject in the plurality of reference subjects has a non-zero tumor fraction. In some embodiments, at least 50% of the reference subject in the plurality of reference subjects each have a tumor fraction at least 0.1, at least 0.2, at least 0.3, at least 0.4, or at least 0.5.

In some embodiments, each respective probe in the plurality of probes includes a respective nucleic acid sequence that is complementary or substantially complementary to the respective genomic region (see e.g., descriptions with regard to blocks 222 and 224 above).

In some embodiments, the reference model comprises a linear regression model (e.g., as described above with regard to block 250). In some embodiments, the reference model is a multivariate logistic regression, a neural network, a convolutional neural network, a support vector machine (SVM), a decision tree, a regression algorithm, or a supervised clustering model, as discussed above.

In some embodiments, the corresponding first biological sample of each respective reference subject comprises a liquid sample of the respective reference subject (e.g., as described above with regard to block 218).

In some embodiments, the corresponding first biological sample of the respective reference subject comprises a corresponding first plurality of cell-free nucleic acids, where the corresponding first plurality of cell-free nucleic acids comprises at least 1000 cell-free nucleic acids that are aligned to a reference genome of the reference subject. In some embodiments, the corresponding first plurality of cell-free nucleic acids comprises at least 1000, at least 2000, at least 3000, at least 4000, at least 5000, at least 6000, at least 7000, at least 8000, at least 9000, at least 10,000, at least 20,000, at least 50,000, or at least 100,000 cell-free nucleic acids that are aligned to the reference genome of the species.

In some embodiments, the corresponding plurality of bin values for each respective reference subject is derived by using the corresponding first plurality of sequence reads to determine a respective number of unique nucleic acid fragments represented by the corresponding first plurality of sequence reads that map to each respective bin in the plurality of bins, thereby determining each respective bin value in the corresponding plurality of bin values.

In some embodiments, bin values as used in the method are normalized from raw sequence read counts in various ways (e.g., correction of systematic errors, correction of GC biases, correction of biases due to PCR over-amplification, etc.), for example as described in the section entitled Determining bin values from counts of sequence reads. In some embodiments, bin values indicate copy number instability (CNI) or copy number changes, for example as described above with reference to block 208.

In some embodiments, the respective corresponding region of the reference genome, or a portion thereof, of each corresponding bin in a first set of bins in the plurality of bins is complementary or substantially complementary to the sequences of two or more probes in a plurality of probes used in a targeted nucleic acid sequencing to generate the plurality of bin values (e.g., on-target regions, such as genes). In some embodiments, the respective corresponding region of the reference genome, or a portion thereof, for each corresponding bin in a second set of bins in the plurality of bins is not represented by a sequence of any in the plurality of probes (e.g., is off-target, intergenic regions). See block 210 for a description of bin sizes and mapping sequence reads to bins.

In some embodiments, the corresponding first biological sample of the respective reference subject comprises a corresponding first plurality of cell-free nucleic acids, where the corresponding first plurality of cell-free nucleic acids comprises at least 1000 cell-free nucleic acids that are aligned to a reference genome of the reference subject. In some such embodiments, the plurality of allele frequencies for each respective reference subject are derived by using the corresponding second plurality of sequence reads to identify support for an allele for a variant in a variant set, thereby determining an observed frequency of the allele for the variant in the variant set, where each observed frequency corresponds to a respective allele frequency in the plurality of allele frequencies.

In some embodiments, the plurality of allele frequencies for each respective reference subject is derived as described above with respect to blocks 238-242.

In some embodiments, for example as described above with reference to block 216, a respective sequence read in the corresponding second plurality of sequence reads is deemed to support an allele of a first variant in the variant set when the respective sequence read corresponds to the allele of the first variant. In some embodiments, a respective sequence read in the corresponding second plurality of sequence reads is deemed not to support an allele of the first variant in the variant set when the respective sequence read does not contain the allele of the first variant. In some embodiments, the observed frequency of the first variant is determined by a ratio or proportion between (i) a corresponding first number of unique cell-free nucleic acids, represented by the corresponding second plurality of sequence reads, that support the allele of the first variant and (ii) a corresponding second number of unique cell-free nucleic acids, represented by the corresponding second plurality of sequence reads, that map to the genomic region encompassing the allele irrespective of whether they support or do not support the allele, where the corresponding second number of unique cell-free nucleic acids includes the corresponding first number of cell-free nucleic acids.

In some embodiments, determining the plurality of copy number values b) comprises, for each respective reference subject in the plurality of subjects, applying a dimensionality reduction method as described herein to the plurality of bin values, thereby identifying all or a subset of the plurality of features in the form of a plurality of dimension reduction components.

In some embodiments, the tumor fraction of each respective reference subject in the plurality of reference subjects is between 0.001 and 1.0. In some embodiments, the range of tumor fraction of each respective reference subject comprises the range described above in reference to block 250.

The Cancer Genome Atlas (TCGA) Study.

In some embodiments, genotypic information is obtained using data from the Cancer Genome Atlas (TCGA) cancer genomics program that is led by the National Cancer Institute and the National Human Genome Research Institute. The TCGA dataset comprises, among other information, gene expression profiles from dissected tissue samples of a large number of human cancer samples. The information is obtained using high-throughput platforms including gene expression mutation, copy number, methylation, etc. The TCGA dataset is a publicly available dataset comprising more than two petabytes of genomic data for over 11,000 cancer patients, including clinical information about the cancer patients, metadata about the samples (e.g., the weight of a sample portion, etc.) collected from such patients, histopathology slide images from sample portions, and molecular information derived from the samples (e.g., mRNA/miRNA expression, protein expression, copy number, etc.). The TCGA dataset includes array-based sequencing data obtained using genome-wide array analysis using the Genome-Wide Human SNP Array 6.0 from Affymetrix for subjects. The TCGA dataset includes such data for subjects with a known particular cancer and the data for each respective subject is from the isolated and pure tissue originating the cancer in the respective subject. A total of 33 different cancers are represented in the TCGA dataset: breast (breast ductal carcinoma, bread lobular carcinoma) central nervous system (glioblastoma multiforme, lower grade glioma), endocrine (adrenocortical carcinoma, papillary thyroid carcinoma, paraganglioma & pheochromocytoma), gastrointestinal (cholangiocarcinoma, colorectal adenocarcinoma, esophageal cancer, liver hepatocellular carcinoma, pancreatic ductal adenocarcinoma, and stomach cancer), gynecologic (cervical cancer, ovarian serous cystadenocarcinoma, uterine carcinosarcoma, and uterine corpus endometrial carcinoma), head and neck (head and neck squamous cell carcinoma, uveal melanoma), hematologic (acute myeloid leukemia, Thymoma), skin (cutaneous melanoma), soft tissue (sarcoma), thoracic (lung adenocarcinoma, lung squamous cell carcinoma, and mesothelioma), and urologic (chromophobe renal cell carcinoma, clear cell kidney carcinoma, papillary kidney carcinoma, prostate adenocarcinoma, testicular germ cell cancer, and urothelial bladder carcinoma). See Blum et al., 2018, “TCGA-Analyzed Tumors,” SNAPSHOT 173(2), P530, which is hereby incorporated by reference.

The Circulating Cell-Free Genome Atlas (CCGA) Study.

Subjects from the CCGA Study were used in the present disclosure. The CCGA (NCT02889978) study is a prospective, multi-center, observational cfDNA-based, case-control early cancer detection study that has enrolled 15,254 demographically-balanced participants (44% non-cancer, 56% cancer) from 142 sites in North America with longitudinal follow-up, designed to develop a single blood test for 50+ cancer types across cancer stages. See, Liu et al., “Sensitive and specific multi-cancer detection and localization using methylation signatures in cell-free DNA,” Ann. Oncol2020, https://doi.org/10.1016/j.annonc.2020.02.011, which is hereby incorporated by reference. The CCGA study includes a plasma cell-free DNA (cfDNA)-based multi-cancer detection assay. Up to 80 ml of whole blood was collected from subjects with newly diagnosed therapy-naive cancer (C, case) and participants without a diagnosis of cancer (noncancer [NC], control) as defined at enrollment.

All samples were analyzed by: 1) paired cfDNA and white blood cell (WBC)-targeted sequencing (60,000×, 507 gene panel, herein referred to as the “ART” panel); a joint caller removed WBC-derived somatic variants and residual technical noise; 2) paired cfDNA and WBC whole-genome sequencing (WGS; 35×); a novel machine learning algorithm generated cancer-related signal scores; joint analysis identified shared events; and 3) cfDNA whole-genome bisulfite sequencing (WGBS; 34×); normalized scores were generated using abnormally methylated fragments. Details of WBC sequencing and analysis are provided in U.S. patent application Ser. No. 16/201,912, entitled “Models for Targeted Sequencing,” filed on Nov. 27, 2018. WBC sequence analysis enables both removal of somatic variants that are non-cancer related and identification of cancer-related somatic variants. First, by comparing paired cfDNA variants with WBC variants from a single subject, somatic variants that are not related to cancer (e.g., those found in the WBC sequences and in the cfDNA sequences) can be identified. This constitutes a background normalization of the subject's sequencing information (e.g., by removing non-cancer somatic variants from further analysis). Second, by comparing WBC variants from a subject with WBC variants from NC subjects, somatic variants that may be cancer-related are identified (e.g., and retained for downstream analysis).

In the targeted assay, non-tumor WBC-matched cfDNA somatic variants (SNVs/indels) accounted for 76% of all variants in NC and 65% in C. Consistent with somatic mosaicism (e.g., clonal hematopoiesis), WBC-matched variants increased with age; several were non-canonical loss-of-function mutations not previously reported. After WBC variant removal, canonical driver somatic variants were highly specific to C (e.g., in EGFR and PIK3CA, 0 NC had variants vs 11 and 30, respectively, of C). Similarly, of 8 NC with somatic copy number alterations (SCNAs) detected with WGS, four were derived from WBCs. WGBS data of the CCGA reveals informative hyper- and hypo-fragment level CpGs (1:2 ratio); a subset of which was used to calculate methylation scores. A consistent “cancer-like” signal was observed in <1% of NC participants across all assays (e.g., representing potentially undiagnosed cancers). An increasing trend was observed in NC vs stages I-III vs stage IV (nonsyn, SNVs/indels per Mb [Mean±SD] NC: 1.01±0.86, stages I-III: 2.43±3.98; stage IV: 6.45±6.79; WGS score NC: 0.00±0.08, I-III: 0.270.98; IV: 1.95±2.33; methylation score NC: 0±0.50; I-III: 1.02±1.77; IV: 3.94±1.70). These data demonstrate the feasibility of achieving >99% specificity for invasive cancer, and support the promise of cfDNA assay for early cancer detection.

Determining Bin Values from Counts of Sequence Reads.

In some embodiments, each bin count for a subject is calculated by determining a number of fragments represented by sequence reads, obtained from sequencing cell-free nucleic acids from the subject, that correspond to a respective bin. Each bin in the plurality of bins represents a portion of a reference genome of the species of the subject. The species can be human, though it should be appreciated that the described methods can be applied to other types of species.

Bin counts in a plurality of bin counts of a subject can be obtained in various ways, including using sequence reads, PCR amplicons, and/or microarray technologies that use relative quantitation in which the intensity of a signal (at a spot (e.g., a DNA spot)) is compared to the intensity of the signal of the same spot under a different condition, and the identity of the feature is known by its position. In some embodiments, the plurality of bin counts are determined using any of the techniques disclosed in U.S. Patent Publication No. 2019-0164627 A1 entitled “Models for Targeted Sequencing,” published May 30, 2019 or U.S. Patent Publication No. 2019-0287646 A1 entitled “Identifying Copy Number Aberrations,” published Sep. 19, 2019, which are both hereby incorporated in their entirety.

Any suitable number of cell-free-nucleic acids represented by sequence reads can be used to determine bin counts. For example, in some embodiments, the plurality of bin values of a respective subject is determined using more than 1000, more than 3000, more than 5000, more than 10000, more than 20000, more than 50000, or more than 100000 sequence reads that are collectively taken from a biological sample of the respective subject. In some embodiments, each sequence read used to form the plurality of bin values of a respective subject includes (i) a first portion that is mappable onto the genome of the species and (ii) a second portion (e.g., a UMI). In some embodiments, the sequence reads used to form the plurality of bin counts of a respective subject are filtered so that only sequence reads whose first portion is less than 160 nucleotides are used to form the bin counts.

In some embodiments, each bin count, for a respective subject, is determined from a number of unique nucleic acid fragments) in the cell-free nucleic acid obtained from the first biological sample that map onto the different portion of the genome of the species represented by the respective bin. Depending on the sequencing method used, each such unique nucleic acid fragment may be represented by a number of sequence reads. In some embodiments, this redundancy in sequence reads to unique nucleic acid fragments in the cell-free nucleic acid is resolved using multiplex sequencing techniques such as barcoding so that a bin count for a respective bin represents the number of unique nucleic acid fragments in the cell-free nucleic acid in a biological sample that map onto the different portion of the genome of the species represented by the respective bin, rather than the total number of sequence reads in the plurality of sequence reads mapping to the respective bin. See Kircher et al., 2012, Nucleic Acids Research 40, No. 1 e3, which is hereby incorporated by reference, for example disclosure on barcoding.

In some embodiments, each bin value in a plurality of bin values is representative of genotypic information and corresponds to a number of fragments represented by sequence reads in sequencing information (e.g., bin counts) measured from cell-free nucleic acid in a biological sample of the respective subject. In some embodiments, bin values correspond to bin counts that have undergone at least one form of normalization.

In some embodiments, the sequencing data is pre-processed to correct biases or errors using one or more methods such as normalization, correction of GC biases, correction of biases due to PCR over-amplification, etc.

For instance, In some embodiments, a median bin count across the plurality of bin counts for a respective subject is obtained. In some embodiments, mean bin count can be used instead. Then, each respective bin count in the plurality of bin counts for the respective subject is divided by this median value thus assuring that the bin counts for the respective subject are centered on a known value (e.g., on zero):

$$b {v}_{i}^{*} = \frac{b {v}_{i}}{median ( b {v}_{j} )}$$

where,

bvᵢ=the bin count of bin i in the plurality of bin counts for the respective subject,

bvᵢ*=the normalized bin value of bin i in the plurality of bin values for the respective subject upon this first normalization, and

median(bvⱼ)=the median bin count across the first plurality of unnormalized bin counts for the respective subject.

In some embodiments, rather than using the median bin count across the plurality of bin counts, some other measure of central tendency is used, such as an arithmetic mean, weighted mean, midrange, midhinge, trimean, Winsorized mean, mean, or mode across the plurality of bin counts of the respective subject.

In some embodiments, each respective normalized bin value bvᵢ* is further normalized by the median normalized value for the respective bin across the first plurality of subjects k:

$$b {v}_{i}^{* *} = log ( \frac{b {v}_{i}^{*}}{median ( b {v}_{i k}^{* *} )} )$$

where,

bvᵢ*=the normalized bin value of bin i in the plurality of bin values for the respective subject from the first normalization procedure described above,

bvᵢ**=the normalized bin value of bin i for the respective subject upon this second normalization described here, and

median(bvᵢₖ**)=the median normalized bin value bvᵢ* for bin i across the first plurality of subjects (k subjects).

In some embodiments, the un-normalized bin counts bvᵢ are further corrected for GC bias (e.g., are GC normalized). In some embodiments, the normalized bin values bvᵢ* are further GC normalized. In some embodiments, the normalized bin counts bvᵢ** are further GC normalized. In such embodiments, GC counts of respective sequence reads in the plurality of sequence reads of each subject in a plurality of subjects are binned. A curve describing the conditional mean fragment count per GC value is estimated by such binning (Yoon et al., 2009, Genome Research 19(9):1586), or, alternatively, by assuming smoothness (Boeva et al., 2011, Bioinformatics 27(2), p. 268; Miller et al., 2011, PLoS ONE 6(1), p. e16327). The resulting GC curve determines a predicted value for each bin based on the bin's GC. These predictions can be used directly to normalize the original signal (e.g., bvᵢ*, bvᵢ**, or bvᵢ***). As a non-limiting example, in the case of binning and direct normalization, for each respective G+C percentage in the set {0%, 1%, 2%, 3%, . . . , 100%}, the value m_{GC}, the median value of bvᵢ** of all bins across the first plurality of subjects having this respective G+C percentage, is determined and subtracted from the normalized bin values bvᵢ** of those bins having the respective G+C percentage to form GC normalized bin values bvᵢ***. In FIG. 10, curve 1002 is a plot of G+C percentage versus bin value bvᵢ** across the plurality of bins across the plurality of subjects. Upon GC normalization, GC normalized bin values bvᵢ*** (e.g., as set forth in plot 1004 of FIG. 10) are now centered on GC content, thereby removing GC bias from the bin values. In some embodiments, rather than using the median value of bvᵢ** of all bins across the first plurality of subjects having this respective G+C percentage, some other form of measuring the central tendency of bvᵢ** of all bins across the first plurality of subjects having this respective G+C percentage is used, such as an arithmetic mean, weighted mean, midrange, midhinge, trimean, Winsorized mean, mean, or mode. In some embodiments, curve 1002 of FIG. 10 is determined using a locally weighted scatterplot smoothing model (e.g., LOESS, LOWESS, etc.). See, for example, Benjamini and Speed, 2012, Nucleic Acids Research 40(10): e72; and Alkan et al., 2009, Nat Genet 41:1061-7. For example, in some embodiments, the GC bias curve is determined by LOESS regression of count by GC (e.g., using the ‘loess’ R package) on a random sampling (or exhaustive sampling) of bins from the plurality of subjects. In some embodiments, the GC bias curve is determined by LOESS regression of count by GC (e.g., using the ‘loess’ R package), or some other form of curve fitting, on a random sampling of bins from a cohort of young, healthy subjects that have been sequenced using the same sequencing techniques used to sequence the first plurality of subjects.

In some embodiments, the bin values are further normalized using principal component analysis (PCA) to remove other coverage biases. In some embodiments, these other coverage biases are higher-order artifacts for a population-based correction (e.g., based on a group of healthy subjects). See, for example, Price et al., 2006, Nat Genet 38, pp. 904-909; Leek and Storey, 2007, PLoS Genet 3, pp. 1724-1735; and Zhao et al., 2015, Clinical Chemistry 61(4), pp. 608-616. Such normalization can be in addition to or instead of any of the above-identified normalization techniques. In some such embodiments, to train the PCA normalization, a data matrix comprising LOESS normalized bin values bvᵢ*** from young, healthy subjects in the first plurality of subjects (or another cohort that was sequenced in the same manner as the first plurality of subjects) is used and the data matrix is transformed into principal component space thereby obtaining the top N number of principal components across the training set. In some embodiments, the top 2, the top 3, the top 4, the top 5, the top 6, the top 7, the top 8, the top 9 or the top 10 such principal components are used to build a linear regression model:

bvᵢ***˜LM(PC₁, . . . ,PC_{N})

Then, each bin bvᵢ*** of each respective bin of each respective subject in the first plurality of subjects is fit to this linear model to form a corresponding PCA-normalized bin value bvᵢ****:

bvᵢ****=bvᵢ***−fit_{LM(PC}_{₁}_{, . . . ,PC}_{_{N}}₎.

In other words, for each respective subject in the plurality of subjects, a linear regression model is fit between its normalized bin values {bv₁***, . . . , bvᵢ***} and the top principal components from the training set, where K is the total number of bin values in the plurality of bin values. The residuals of this model serve as final normalized bin values {bvᵢ****, . . . , bvᵢ****} for the respective subject. Intuitively, the top principal components represent predictable bias commonly seen in healthy samples, and therefore removing such noise (in the form of the top principal components derived from the healthy cohort) from the bin values bvᵢ*** can effectively improve normalization. See Zhao et al., 2015, Clinical Chemistry 61(4), pp. 608-616 for further disclosure on PCA normalization of sequence reads using a health population. Regarding the above normalization, it will be appreciated that all variables are standardized (e.g., by subtracting their means and dividing by their standard deviations) when necessary.

It will be appreciated that, through the present disclosure, the term “bin count” refers to any un-normalized form of representation of the number of nucleic fragments mapping to a given bin i (e.g., bvᵢ). Through the present disclosure, the term “bin value” refers to normalized forms of bin counts (e.g., bvᵢ*, bvᵢ**, bvᵢ***, bvᵢ****, etc.).

### Example Bins for Methylation Embodiments

In some embodiments the bins of the present disclosure are designed to encompass only targeted regions of the human genome that have cancer- and/or tissue-specific methylation patterns. This example summarizes the identification of suitable regions of the human genome to be encompassed by such bins. Based on the results of the above described CCGA study, as further described in Liu et al., “Sensitive and specific multi-cancer detection and localization using methylation signatures in cell-free DNA,” Ann. Oncol 2020, doi.org/10.1016/j.annonc.2020.02.011, the portions of the human genome (the hg19 genome, Vogelstin et al., 2013, “Cancer genome landscapes,” Science 339 1546-1558) predicted to contain cancer- and/or tissue-specific methylation patterns in cfDNA relative to non-cancer controls were identified and the most informative regions selected to be represented by the bins of some embodiments of the present disclosure.

Specifically, after bisulfite treatment, targeted cfDNA fragments containing abnormal methylation patterns relative to non-cancer controls from both strands were enriched using biotinylated probes. Briefly, 120-bp biotinylated DNA probes were designed to target enrichment of bisulfite-converted DNA from either hypermethylated fragments (100% methylated CpGs) or hypomethylated fragments (100% unmethylated CpGs); probes tiled target regions with 50% overlap between adjacent probes. A custom algorithm aligned candidate probes to the genome and scored the number of on- and off-target mapping events. Probes with elevated off-target mapping were omitted from the final panel of regions to be represented by the bins of some embodiments of the present disclosure.

As disclosed in U.S. patent application Ser. No. 15/931,022, entitled “Model Based Featurization and Classification,” filed May 13, 2020, a targeted methylation panel, all or a portion of which is represented by the bins of some embodiments of the present disclosure, covering 103,456 distinct regions (17.2 Mb), covering 1,116,720 CpGs was identified using the whole genome bisulfite data obtained from CCGA sub-study CCGA-1. This included 363,033 CpGs in 68,059 regions (7.5 Mb) covered by probes targeting hypomethylated fragments; 585,181 CpGs in 28,521 regions (7.4 Mb) covered by probes targeting hypermethylated fragments; and 218,506 CpGs in 6,876 regions (2.3 Mb) targeting both types of fragments. Individual abnormal target regions contained between 1 and 590 CpGs, with a median CpG count of 3 for hypomethylated target regions and 6 for hypermethylated target regions. CpGs were present in the following genomic regions unv the nomenclature of Cavalcante and Sartor, 2017, “annotatr: genomic regions in context,” Bioinformatics 33(15):2381-2383: 193,818 (17%) in the region 1 to 5 kbp upstream of transcription start sites (TSSs); 278,872 (24%) in promoters (<1 kbp upstream of TSSs); 500,996 (43%) in introns; 292,789 (25%) in exons; 247,752 (21%) in intron-exon boundaries (i.e., 200 bp up- or down-stream of any boundary between an exon and intron; boundaries are with respect to the strand of the gene); 134,144 (11%) in 5′-untranslated regions; 28,388 (2.4%) in 3′-untranslated regions; 182,174 (16%) between genes; and the remaining 1,817 (<1%) were not annotated. Percentages were relative to the total number of CpGs and do not sum to 100% because each CpG could receive multiple annotations due to overlapping genes and/or transcripts.

Cancer Assay Probes and Panels.

In various embodiments, the reference models described herein use samples enriched using a cancer assay panel comprising a plurality of probes or a plurality of probe pairs. A number of targeted cancer assay panels are known in the art, for example, as described in WO 2019/195268 entitled “Methylation Markers and Targeted Methylation Probe Panels,” filed Apr. 2, 2019, PCT/US2019/053509, filed Sep. 27, 2019 and PCT/US2020/015082 entitled “Detecting Cancer, Cancer Tissue or Origin, or Cancer Type,” filed Jan. 24, 2020 (which are each incorporated by reference herein in their entirety). For example, in some embodiments, the cancer assay makes use of a plurality of probes (or probe pairs) that can capture fragments (cell-free nucleic acids) that can together provide information relevant to determination of tumor fraction and/or diagnosis of cancer. In some embodiments, a panel of probes includes at least 50, 100, 500, 1,000, 2,000, 2,500, 5,000, 6,000, 7,500, 10,000, 15,000, 20,000, 25,000, or 50,000 pairs of probes. In other embodiments, a panel of probes includes at least 500, 1,000, 2,000, 5,000, 10,000, 12,000, 15,000, 20,000, 30,000, 40,000, 50,000, or 100,000 probes. The plurality of probes together can comprise at least 0.1 million, 0.2 million, 0.4 million, 0.6 million, 0.8 million, 1 million, 2 million, 3 million, 4 million, 5 million, 6 million, 7 million, 8 million, 9 million, or 10 million nucleotides. The probes (or probe pairs) are specifically designed to target one or more genomic regions differentially methylated in cancer and non-cancer samples. The target genomic regions can be selected to maximize classification accuracy, subject to a size budget (which is determined by sequencing budget and desired depth of sequencing).

Samples enriched using a cancer assay panel can be subject to targeted sequencing. Samples enriched using the cancer assay panel can be used to determine tumor fraction, determine presence of absence of cancer generally and/or provide a cancer classification such as cancer type, stage of cancer such as I, II, III, or IV, or provide the tissue of origin where the cancer is believed to originate. Depending on the purpose, a panel can include probes (or probe pairs) targeting genomic regions differentially methylated between general cancerous (pan-cancer) samples and non-cancerous samples, or only in cancerous samples with a specific cancer type (e.g., lung cancer-specific targets). Specifically, a cancer assay panel is designed based on bisulfite sequencing data generated from the cell-free DNA (cfDNA) or genomic DNA (gDNA) from cancer and/or non-cancer individuals.

In some embodiments, the panel of probes designed by methods provided herein comprises at least 1,000 pairs of probes, each pair of which comprises two probes configured to overlap each other by an overlapping sequence comprising a 30-nucleotide fragment. The 30-nucleotide fragment comprises at least five CpG sites, where at least 80% of the at least five CpG sites are either CpG or UpG. The 30-nucleotide fragment is configured to bind to one or more genomic regions in cancerous samples, where the one or more genomic regions have at least five methylation sites with an abnormal methylation pattern. Another panel of probes in accordance with the present disclosure comprises at least 2,000 probes, each of which is designed as a hybridization probe complimentary to one or more genomic regions. Each of the genomic regions is selected based on the criteria that it comprises (i) at least 30 nucleotides, and (ii) at least five methylation sites, where the at least five methylation sites have an abnormal methylation pattern and are either hypomethylated or hypermethylated.

Each of the probes (or probe pairs) is designed to target one or more target genomic regions. The target genomic regions are selected based on several criteria designed to increase selective enriching of relevant cfDNA fragments while decreasing noise and non-specific bindings. For example, a panel can include probes that can selectively bind and enrich cfDNA fragments that are differentially methylated in cancerous samples. In this case, sequencing of the enriched fragments can provide information relevant to determination of tumor fraction or diagnosis of cancer. Furthermore, the probes can be designed to target genomic regions that are determined to have an abnormal methylation pattern and/or hypermethylation or hypomethylation patterns to provide additional selectivity and specificity of the detection. For example, genomic regions can be selected when the genomic regions have a methylation pattern with a low p-value according to a Markov model trained on a set of non-cancerous samples, and when the genomic regions additionally cover at least 5 CpGs, 90% of which are either methylated or unmethylated. In other embodiments, genomic regions can be selected utilizing mixture models, as described herein.

Each of the probes (or probe pairs) can target genomic regions comprising at least 25 bp, 30 bp, 35 bp, 40 bp, 45 bp, 50 bp, 60 bp, 70 bp, 80 bp, or 90 bp. The genomic regions can be selected by containing less than 20, 15, 10, 8, or 6 methylation sites. The genomic regions can be selected when at least 80, 85, 90, 92, 95, or 98% of the at least five methylation (e.g., CpG) sites are either methylated or unmethylated in non-cancerous or cancerous samples.

Genomic regions may be further filtered to select only those that are likely to be informative based on their methylation patterns, for example, CpG sites that are differentially methylated between cancerous and non-cancerous samples (e.g., abnormally methylated or unmethylated in cancer versus non-cancer). For the selection, calculation can be performed with respect to each CpG site. In some embodiments, a first count is determined that is the number of cancer-containing samples (cancer_count) that include a fragment overlapping that CpG, and a second count is determined that is the number of total samples containing fragments overlapping that CpG (total). Genomic regions can be selected based on criteria positively correlated to the number of cancer-containing samples (cancer_count) that include a fragment overlapping that CpG, and inversely correlated with the number of total samples containing fragments overlapping that CpG (total).

In some embodiments filtration is used to select target genomic regions that have off-target genomic regions less than a threshold value. For example, a genomic region is selected only when there are less than 15, 10, or 8 off-target genomic regions. In other cases, filtration is performed to remove genomic regions when the sequence of the target genomic regions appears more than 5, 10, 15, 20, 25, or 30 times in a genome. Further filtration can be performed to select target genomic regions when a sequence, 90%, 95%, 98% or 99% homologous to the target genomic regions, appear less than 15, 10 or 8 times in a genome, or to remove target genomic regions when the sequence, 90%, 95%, 98% or 99% homologous to the target genomic regions, appear more than 5, 10, 15, 20, 25, or 30 times in a genome. This is for excluding repetitive probes that can pull down off-target fragments, which are not desired and can impact assay efficiency.

In some embodiments, fragment-probe overlap of at least 45 bp was demonstrated to be required to achieve a non-negligible amount of pulldown (though this number can be different depending on assay details). Furthermore, it has been suggested that more than a 10% mismatch rate between the probe and fragment sequences in the region of overlap is sufficient to greatly disrupt binding, and thus pulldown efficiency. Therefore, sequences that can align to the probe along at least 45 bp with at least a 90% match rate are candidates for off-target pulldown. Thus, in some embodiments, the number of such regions is scored. The best probes have a score of 1, meaning they match in only one place (the intended target region). Probes with a low score (say, less than 5 or 10) are accepted, but any probes above the score are discarded. Other cutoff values can be used for specific samples.

In various embodiments, the selected target genomic regions can be located in various positions in a genome, including but not limited to exons, introns, intergenic regions, and other parts. In some embodiments, probes targeting non-human genomic regions, such as those targeting viral genomic regions, can be added.

Select Human Genomic Regions Used for Bins.

In some embodiments of the present disclosure, each bin in the plurality of bins is drawn from a panel of genomic regions that is designed for targeted selection of cancer-specific methylation patterns. In some embodiments, each such genomic region is drawn from Table 2 of International Patent Application No. PCT/US2020/015082, entitled “Detecting Cancer, Cancer Tissue or Origin, or Cancer Type,” filed Jan. 24, 2020, which is hereby incorporated by reference, including the Sequence Listing referenced therein.

SEQ ID NOs 452,706-483,478 of PCT/US2020/015082 provide further information about certain hypermethylated or hypomethylated target genomic regions. These SEQ ID NO records identify target genomic regions that can be differentially methylated in samples from specified pairs of cancer types. The target genomic regions of SEQ ID NOs 452,706-483,478 of PCT/US2020/015082 are drawn from list 6 of PCT/US2020/015082. Many of the same target genomic regions are also found in lists 1-5 and 7-16 of PCT/US2020/015082. The entry for each SEQ ID indicates the chromosomal location of the target genomic region relative to hg19, whether cfDNA fragments to be enriched from the region are hypermethylated or hypomethylated, the sequence of one DNA strand of the target genomic region, and the pair or pairs of cancer types that are differentially methylated in that genomic region. As the methylation status of some target genomic regions distinguish more than one pair of cancer types, each entry identifies a first cancer type as indicated in TABLE 3 of PCT/US2020/015082, including the Sequence Listing referenced therein and one or more second cancer types.

In some embodiments, the plurality of bins of the present disclosure includes a separate bin for each of at least 200, 500, 1,000, 5,000, 10,000, 15,000, 20,000, 30,000, 40,000, or 50,000 target genomic regions in any one of lists 1-16, lists 1-3, lists 13-16, list 12, list 4, or lists 8-11 of PCT/US2020/015082. In some embodiments, the plurality of bins of the present disclosure includes a separate bin for each of at least 200, 500, 1,000, 5,000, 10,000, 15,000, 20,000, 30,000, 40,000, or 50,000 target genomic regions in any combination of one or more lists 1-16 of PCT/US2020/015082 (e.g., such as lists 1-3, lists 13-16, list 12, list 4, or lists 8-11).

In some embodiments, the plurality of bins of the present disclosure includes a separate bin for each of at least 20%, 30%, 40%, 50%, 60%, 70%, 80%, 90%, or 95% of the target genomic regions in any one of lists 1-16 of PCT/US2020/015082. In some embodiments, the plurality of bins of the present disclosure includes a separate bin for each of at least 20%, 30%, 40%, 50%, 60%, 70%, 80%, 90% or 95% of the target genomic regions in any combination of one or more lists 1-16 of PCT/US2020/015082 (e.g., such as lists 1-3, lists 13-16, list 12, list 4, or lists 8-11).

Additional Select Human Genomic Regions Used for Bins.

In some embodiments of the present disclosure, each bin in the plurality of bins is drawn from a panel of genomic regions that is designed for targeted selection of cancer-specific methylation patterns. In some embodiments, each such genomic region is drawn from Table 2 of International Patent Application No. PCT/US2019/053509, published as WO2020/669350A1, entitled “Methylated Markers and Targeted Methylation Probe Panel,” filed Sep. 27, 2019, which is hereby incorporated by reference, including the Sequence Listing referenced therein.

The sequence listing of WO2020/669350A1 includes the following information: (1) SEQ ID NO, (2) a sequence identifier that identifies (a) a chromosome or contig on which the CpG site is located and (b) a start and stop position of the region, (3) the sequence corresponding to (2) and (4) whether the region was included based on its hypermethylation or hypomethylation score. The chromosome numbers and the start and stop positions are provided relative to a known human reference genome, GRCh37/hg19. The sequence of GRCh37/hg19 is available from the National Center for Biotechnology Information (NCBI), the Genome Reference Consortium, and the Genome Browser provided by Santa Cruz Genomics Institute.

Generally, a bin can encompass any of the CpG sites included within the start/stop ranges of any of the targeted regions included in lists 1-8 of WO2020/069350.

In some embodiments, the plurality of bins of the present disclosure includes a separate bin for each of at least 200, 500, 1,000, 5,000, 10,000, 15,000, 20,000, 30,000, 40,000, or 50,000 target genomic regions in any one of lists 1-8 of WO2020/069350. In some embodiments, the plurality of bins of the present disclosure includes a separate bin for each of at least 200, 500, 1,000, 5,000, 10,000, 15,000, 20,000, 30,000, 40,000, or 50,000 target genomic regions in any combination of lists 1-8 of WO2020/069350.

In some embodiments, the plurality of bins of the present disclosure includes a separate bin for each of at least 20%, 30%, 40%, 50%, 60%, 70%, 80%, 90%, or 95% of the target genomic regions in any one of lists 1-8 of WO2020/069350. In some embodiments, the plurality of bins of the present disclosure includes a separate bin for each of at least 20%, 30%, 40%, 50%, 60%, 70%, 80%, 90% or 95% of the target genomic regions in any combination of lists 1-8 of WO2020/069350.

Additional Select Human Genomic Regions Used for Bins.

In some embodiments of the present disclosure, each bin in the plurality of bins is drawn from a panel of genomic regions that is designed for targeted selection of cancer-specific methylation patterns. In some embodiments, each such bin corresponds to a genomic region in any of Table 1-24 of International Patent Application No. PCT/US2019/025358, published as WO2019/195268A2, entitled “Methylated Markers and Targeted Methylation Probe Panels,” filed Apr. 2, 2019, which is hereby incorporated by reference.

In some embodiments, each bin of the present disclosure maps to a genomic region listed in one or more of Table 1, 2, 3, 4, 5, 6, 7, 8, 9, 10, 11, 12, 13, 14, 15, 16, 17, 18, 19, 20, 21, 22, 23 and/or 24 of WO2019/195268A2.

In some embodiments, an entirety of plurality of the bins of the present disclosure together are configured to map to at least 30%, 40%, 50%, 60%, 70%, 80%, 90% or 95% of the genomic regions in one or more of Tables 1-24 of WO2019/195268A2. In some such embodiments, each bin in the plurality of bins maps to a single unique corresponding genomic region in any of Tables 1-24 of WO2019/195268A2. In some such embodiments, a bin in the plurality of bins maps of the present disclosure map to one, two, three, four, five, six, seven, eight, nine or ten unique corresponding genomic region in any combination of Tables 1-24 of WO2019/195268A2.

In some such embodiments, each bin in the plurality of bins of the present disclosure maps to a single unique corresponding genomic region in any of Tables 2-10 or 16-24 of WO2019/195268A2. In some such embodiments, a bin in the plurality of bins maps to one, two, three, four, five, six, seven, eight, nine or ten unique corresponding genomic region in any combination of Tables 2-10 or 16-24 of WO2019/195268A2.

In some embodiments, bins the plurality of bins of the present disclosure together are configured to map to at least 30%, 40%, 50%, 60%, 70%, 80%, 90% or 95% of the genomic regions in Tables 1, 2, 3, 4, 5, 6, 7, 8, 9, 10, 11, 12, 13, 14, 15, 16, 17, 18, 19, 20, 21, 22, 23, and/or 24 of WO2019/195268A2.

Protocol for Obtaining Methylation Information from Sequence Reads of Fragments in a Biological Sample.

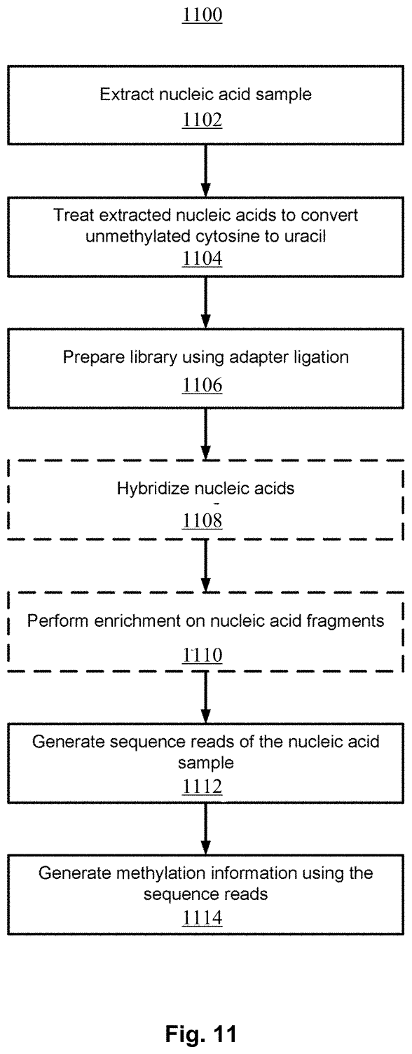

FIG. 11 is a flowchart describing a process 1100 of sequencing fragments (cell-free nucleic acids) and determining methylation states for one or more CpG sites in sequenced fragments, according to some embodiments of the present disclosure. In some embodiments, a methylation state vector is identified for each fragment (cell-free nucleic acid).

In step 1102, nucleic acid (e.g., DNA or RNA) is extracted from a corresponding biological sample of a respective subject. In the present disclosure, DNA and RNA can be used interchangeably unless otherwise indicated. However, the examples described herein can focus on DNA for purposes of clarity and explanation. The biological sample can include nucleic acid molecules derived from any subset of the human genome, including the whole genome. The biological sample can include blood, plasma, serum, urine, fecal, saliva, other types of bodily fluids, or any combination thereof. In some embodiments, methods for drawing a blood sample (e.g., syringe or finger prick) can be less invasive than procedures for obtaining a tissue biopsy, which can require surgery. The extracted sample can comprise cfDNA and/or ctDNA. If a subject has a disease state, such as cancer, cell free nucleic acids (e.g., cfDNA) in an extracted sample from the subject generally includes detectable level of the nucleic acids that can be used to assess a disease state.

In step 1104, the extracted nucleic acids (e.g., including cfDNA fragments) are treated to convert unmethylated cytosines to uracils. In some embodiments, the method 1100 uses a bisulfite treatment of the samples that converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, e.g., APOBEC-Seq (NEBiolabs, Ipswich, Mass.).

In step 1106, a sequencing library is prepared. In some embodiments, the preparation includes at least two steps. In a first step, an ssDNA adapter is added to the 3′-OH end of a bisulfite-converted ssDNA molecule using an ssDNA ligation reaction. In some embodiments, the ssDNA ligation reaction uses CircLigase II (Epicentre) to ligate the ssDNA adapter to the 3′-OH end of a bisulfite-converted ssDNA molecule, where the 5′-end of the adapter is phosphorylated and the bisulfite-converted ssDNA has been dephosphorylated (e.g., the 3′ end has a hydroxyl group). In another embodiment, the ssDNA ligation reaction uses Thermostable 5′ AppDNA/RNA ligase (available from New England BioLabs (Ipswich, Mass.)) to ligate the ssDNA adapter to the 3′-OH end of a bisulfite-converted ssDNA molecule. In this example, the first UMI adapter is adenylated at the 5′-end and blocked at the 3′-end. In another embodiment, the ssDNA ligation reaction uses a T4 RNA ligase (available from New England BioLabs) to ligate the ssDNA adapter to the 3′-OH end of a bisulfite-converted ssDNA molecule.

In a second step, a second strand DNA is synthesized in an extension reaction. For example, an extension primer, which hybridizes to a primer sequence included in the ssDNA adapter, is used in a primer extension reaction to form a double-stranded bisulfite-converted DNA molecule. Optionally, in some embodiments, the extension reaction uses an enzyme that is able to read through uracil residues in the bisulfite-converted template strand.

Optionally, in a third step, a dsDNA adapter is added to the double-stranded bisulfite-converted DNA molecule. Then, the double-stranded bisulfite-converted DNA can be amplified to add sequencing adapters. For example, PCR amplification using a forward primer that includes a P5 sequence and a reverse primer that includes a P7 sequence is used to add P5 and P7 sequences to the bisulfite-converted DNA. Optionally, during library preparation, unique molecular identifiers (UMI) can be added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment, which provides a way to identify sequence reads that came from the same original fragment in downstream analysis.

In an optional step 1108, the nucleic acids (e.g., fragments) can be hybridized. Hybridization probes (also referred to herein as “probes”) may be used to target, and pull down, nucleic acid fragments informative for disease states. For a given workflow, the probes can be designed to anneal (or hybridize) to a target (complementary) strand of DNA or RNA. The target strand can be the “positive” strand (e.g., the strand transcribed into mRNA, and subsequently translated into a protein) or the complementary “negative” strand. The probes can range in length from 10s, 100s, or 1000s of base pairs. Moreover, the probes can cover overlapping portions of a target region.

In an optional step 1110, the hybridized nucleic acid fragments are captured and can be enriched, e.g., amplified using PCR. In some embodiments, targeted DNA sequences can be enriched from the library. This is used, for example, where a targeted panel assay is being performed on the samples. For example, the target sequences can be enriched to obtain enriched sequences that can be subsequently sequenced. In general, any known method in the art can be used to isolate, and enrich for, probe-hybridized target nucleic acids. For example, as is well known in the art, a biotin moiety can be added to the 5′-end of the probes (i.e., biotinylated) to facilitate isolation of target nucleic acids hybridized to probes using a streptavidin-coated surface (e.g., streptavidin-coated beads).

In step 1112, sequence reads are generated from the nucleic acid sample, e.g., enriched sequences. Sequencing data can be acquired from the enriched DNA sequences by known means in the art. For example, the method can include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

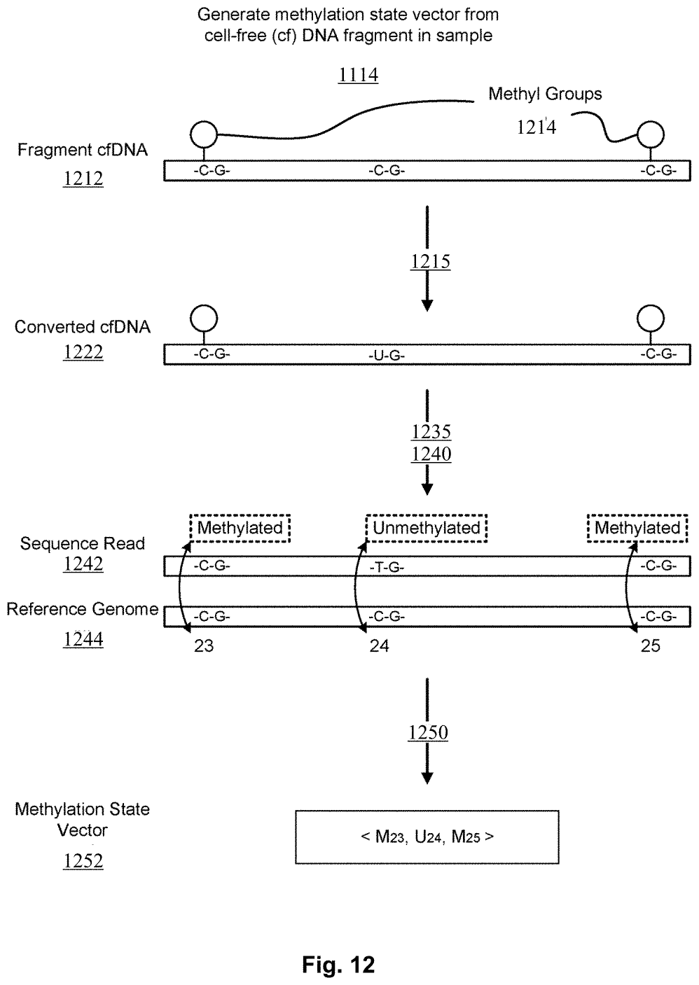

In step 1114, a sequence processor can generate methylation information using the sequence reads. A methylation state vector can then be generated using the methylation information determined from the sequence reads. FIG. 12 is an illustration of the process 1100 of sequencing a cfDNA molecule to obtain a methylation state vector 1252, according to some embodiments of the present disclosure. As an example, a cfDNA fragment is 1212 received that, in this example, contains three CpG sites. As shown, the first and third CpG sites of the cfDNA fragment (molecule) 1212 are methylated 1214. During the treatment step 1215, the cfDNA molecule 1212 is converted to generate a converted cfDNA molecule 1222. During the treatment 1215, the second CpG site, which was unmethylated, has its cytosine converted to uracil. However, the first and third CpG sites were not converted.

After conversion, a sequencing library is prepared 1235 and sequenced 1240, thereby generating a sequence read 1242. The sequence read 1242 is aligned to a reference genome 1244. The reference genome 1244 provides the context as to what position in a human genome the fragment cfDNA originates from. In this simplified example, the analytics system aligns the sequence read 1242 such that the three CpG sites correlate to CpG sites 23, 24, and 25 (arbitrary reference identifiers used for convenience of description). The disclosed systems and methods thus generate information both on methylation status of all CpG sites on the cfDNA fragment (molecule) 1212 and the position in the human genome that the CpG sites map to. As shown, the CpG sites on sequence read 1242, which were methylated, are read as cytosines. In this example, the cytosines appear in the sequence read 1242 only in the first and third CpG site, which allows one to infer that the first and third CpG sites in the original cfDNA molecule were methylated. Whereas, the second CpG site is read as a thymine (U is converted to T during the sequencing process), and thus, one can infer that the second CpG site was unmethylated in the original cfDNA molecule. With these two pieces of information, the methylation status and location, the disclosed systems and methods generate a methylation state vector 1252 for the fragment cfDNA 1212. In this example, the resulting methylation state vector 1252 is <M₂₃, U₂₄, M₂₅>, where M corresponds to a methylated CpG site, U corresponds to an unmethylated CpG site, and the subscript number corresponds to a position of each CpG site in the reference genome.

### Example 1: Correlation of Tumor Fraction with Both Copy Number and Allele Frequency

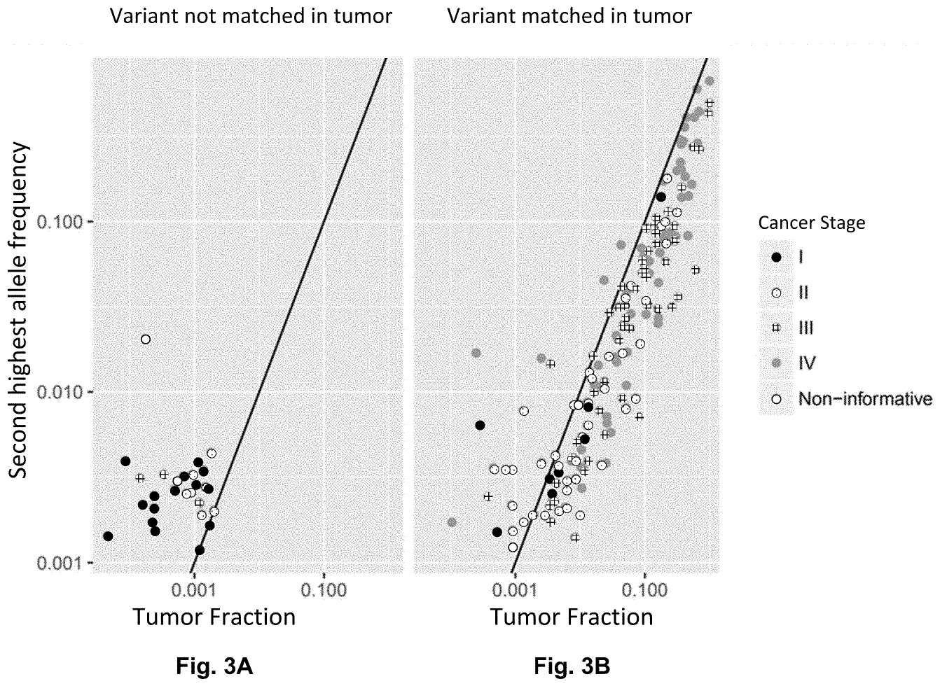

As shown in FIGS. 3A and 3B, tumor fraction is correlated with allele frequency (e.g., the presence of genomic variants). The data in FIGS. 3A and 3B are taken from a CCGA cohort (see CCGA section), where both sequencing data from cell-free nucleic acids and tissue biopsy is available for each patient. In particular, FIG. 3A shows data where the second highest allele frequency (as determined from sequencing of cell-free nucleic acids for a plurality of reference subjects) is not present in the tissue sample. Conversely, FIG. 3B shows data where the second highest allele frequency (as determined from sequencing of cell-free nucleic acids) is present in the tissue sample. In particular, for FIG. 3B (e.g., samples with the matched variant), there is a clear correlation between allele frequency and tumor fraction regardless of the patient's cancer stage, for cases where the tissue data includes the particular allele frequency variant. This demonstrates that, for some patients, an allele frequency is a viable stand-in for tissue sample tumor fraction determinations.

As shown in FIG. 4, tumor fraction can be correlated with both the first and second highest allele frequencies (as calculated across the population of subjects). This is important because variants are not evenly distributed across a population of subjects (i.e., not ever patient has every variant). For example, in FIG. 4, the total number of samples analyzed was 495, with 242 of the samples lacking the first highest allele frequency and another 313 sample lacking the second highest allele frequency. Thus, it is essential to use more than one allele frequency when building a reference model (e.g., to identify multiple allele frequencies that correlate to tumor fraction). In FIG. 4, as in FIGS. 3A and 3B, each known tumor fraction is determined from tissue sample data. In some embodiments, additional allele frequencies beyond the first and second highest are used to determine tumor fraction. For example, in some embodiments, tumor fraction can be correlated with the first highest allele frequency, the second highest allele frequency, the third highest allele frequency, the fourth highest allele frequency, the fifth highest allele frequency, the sixth highest allele frequency, the seventh highest allele frequency, the eighth highest allele frequency, the ninth highest allele frequency, the tenth highest allele frequency, the eleventh highest allele frequency, the twelfth highest allele frequency, the thirteenth highest allele frequency, the fourteenth highest allele frequency, the fifteenth highest allele frequency, the sixteenth highest allele frequency, the seventeenth highest allele frequency, the eighteenth highest allele frequency, the nineteenth highest allele frequency, the twentieth highest allele frequency, or any combination thereof. In some embodiments, tumor fraction can be correlated with any one or more of the top 25 highest allele frequencies, the top 50 highest allele frequencies, or the top 100 highest allele frequencies.

However, despite the observed correlation between allele frequencies and tumor fraction, as displayed in FIGS. 3A, 3B, and 4, there may still be patients who do not have any variants of the genes used to train a tumor fraction reference model present in their cell-free nucleic acid samples. Further, there is a subgroup of the population (e.g., patients akin to those in FIG. 3A) whose tumors will lack the one or more variants within the regions of interest (e.g., bins) that were used to train a reference model.

Therefore, in some embodiments, an additional set of information based on sequence analysis is useful for classification. FIG. 5 illustrates that tumor fraction is also correlated with copy number instability, in accordance with some embodiments of the present disclosure. In FIG. 5, each tumor fraction for a respective patient is determined from a corresponding tissue sample of the respective patient. As with the examples shown in FIG. 4, this correlation holds primarily for subjects (e.g., patients) with a tumor fraction above 0.01.

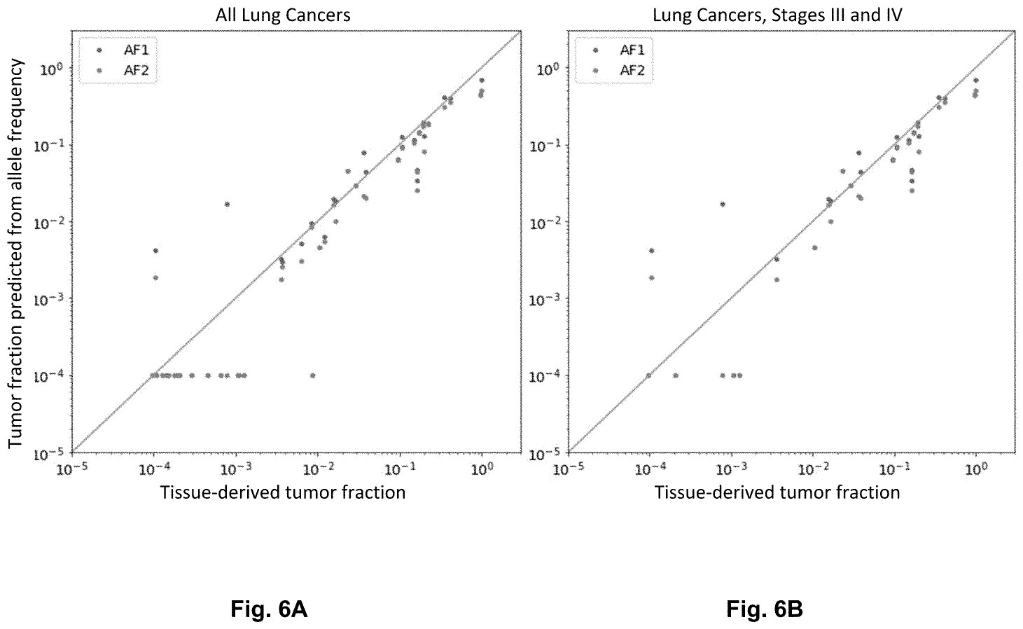

FIGS. 6A and 6B illustrate a particular example of tumor fraction being correlated with allele frequency (e.g., as shown in FIG. 4) for the specific case of patients determined to have lung cancer. Patients with lung cancer from the CCGA study were examined. FIG. 6A include samples from patients with all stages of lung cancer. FIG. 6B is narrowed to those samples that are from just stages III and IV of lung cancer. In both cases, both the first and second highest allele frequencies (as determined by analysis of the lung cancer patients from the CCGA study) are correlated with known tissue-derived tumor fraction for each patient.

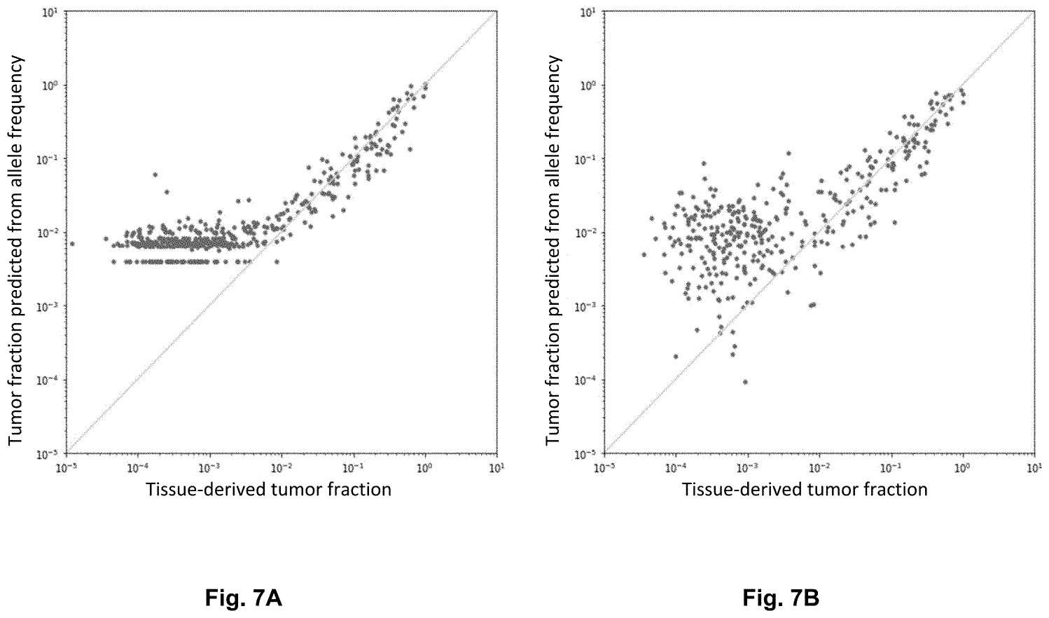

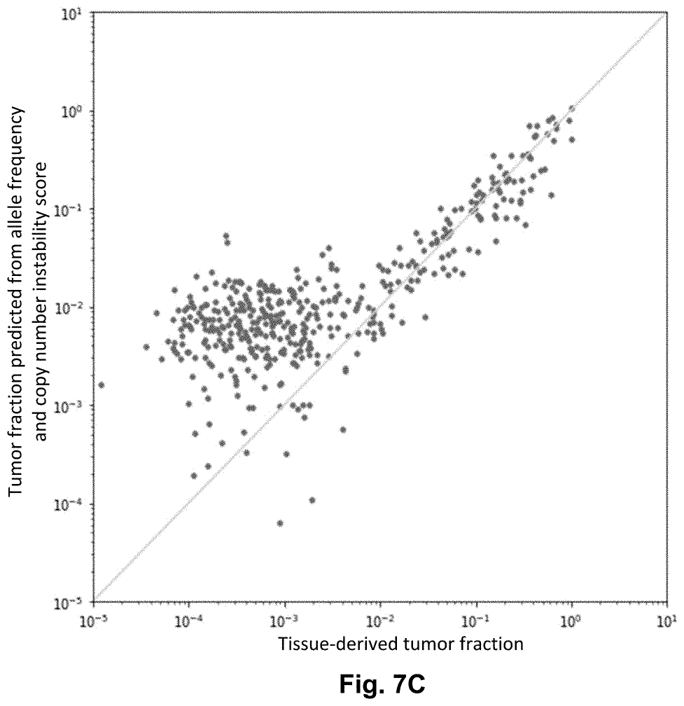

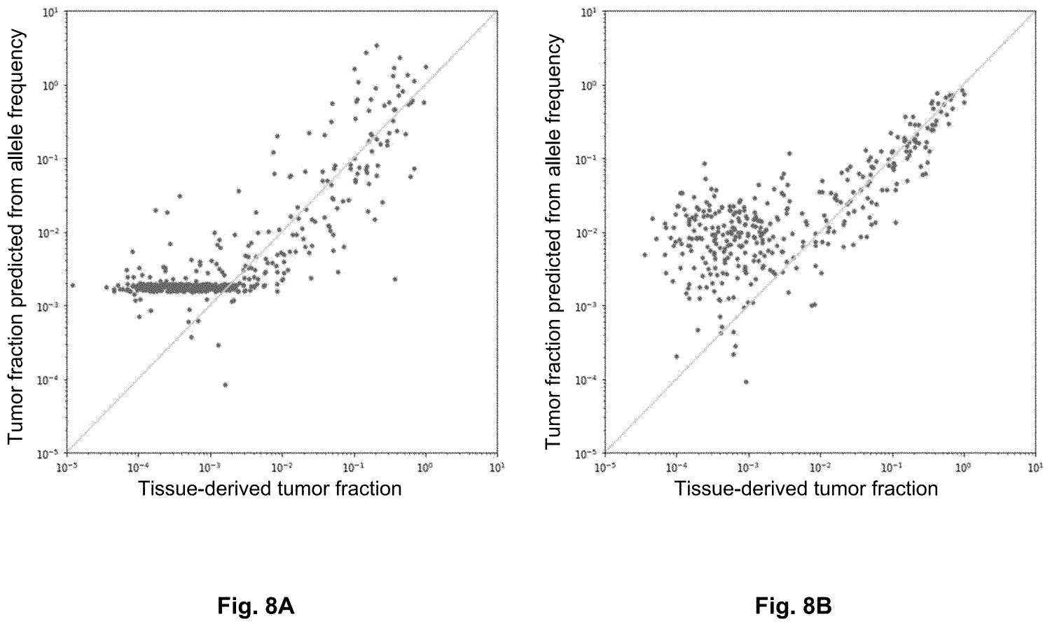

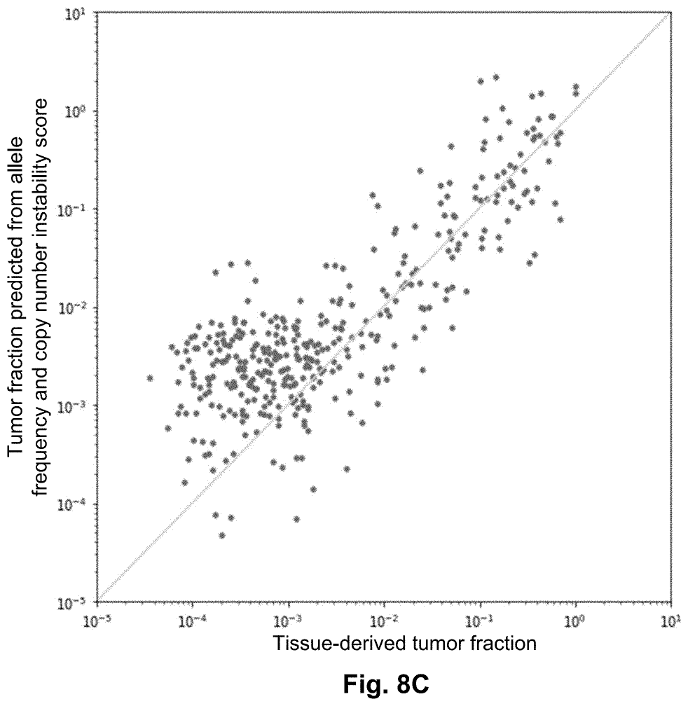

As demonstrated above in FIGS. 4 and 5, respectively, allele frequency and copy number instability are often well correlated with tumor fraction. However, there are instances where the allele frequency for certain allele(s) cannot be determined (e.g., one or more alleles are not present) for a particular patient, or where allele frequency alone does not suffice to determine tumor fraction with sufficient accuracy. Similarly, copy number score alone is not always sufficient to estimate tumor fraction. Collectively, FIGS. 7A-7C and 8A-8C illustrate that a combination of allele frequency and copy number instability correlates, for each patient, with respective tumor fraction estimations determined from corresponding tissue samples. FIG. 7A illustrates the correlation of top 20 allele frequencies per patient with tumor fraction. FIG. 7B illustrates the correlation of copy number score calculated for each subject with tumor fraction. FIG. 7C illustrates that the combination of these metrics results in an improved correlation with tumor fraction. FIG. 8A illustrates the correlation of the top allele frequencies—calculated for each patient—with tumor fraction. FIG. 8B illustrates the correlation of copy number score calculated for each subject with tumor fraction. FIG. 8C illustrates that the combination of these two distinct measurements results in an improved correlation with tumor fraction, and hence an improved predictive model to determine tumor fraction of a subject from cell-free nucleic acids.

### CONCLUSION

Plural instances may be provided for components, operations, or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event (” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The foregoing description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

1. A method of determining a tumor fraction for a subject of a species, the method comprising:
at a computer system comprising at least one processor and a memory storing at least one program for execution by the at least one processor, the at least one program comprising instructions for:
a) obtaining, in electronic form, a first dataset that comprises a plurality of bin values, each respective bin value in the plurality of bin values is for a corresponding bin in a plurality of bins, wherein:
each respective bin in the plurality of bins represents a corresponding region of a reference genome of the species, and
the plurality of bin values is derived from alignment of a first plurality of sequence reads, determined by a first nucleic acid sequencing of a first plurality of cell-free nucleic acids in a first biological sample, to a reference genome of the species, wherein the first biological sample comprises a liquid sample of the subject and the first plurality of cell-free nucleic acids comprises at least 1000 cell-free nucleic acids;
b) determining a plurality of copy number values at least in part from the plurality of bins values;
c) obtaining, in electronic form, a second dataset that comprises a plurality of allele frequencies for a plurality of alleles, wherein:
the plurality of allele frequencies is derived from alignment of a second plurality of sequence reads, determined by a second nucleic acid sequencing of a second plurality of cell-free nucleic acids in a second biological sample, to the reference genome, wherein the second biological sample comprises a liquid sample of the subject and the second plurality of cell-free nucleic acids comprises at least 1000 cell-free nucleic acids; and
d) applying, to a reference model, at least the plurality of copy number values and the plurality of allele frequencies, or a plurality of features derived therefrom, thereby determining the tumor fraction of the subject.

2. The method of claim 1, wherein:
the first biological sample and the second biological sample are a single biological sample,
the first nucleic acid sequencing and the second nucleic acid sequencing is the same nucleic acid sequencing, and
the first plurality of cell-free nucleic acids and the second plurality of cell-free nucleic acids is a single plurality of cell-free nucleic acids.

3. The method of claim 1, wherein:
the first and second nucleic acid sequencing is targeted panel sequencing that provides both the plurality of bin values and the plurality of allele frequencies,
the targeted panel sequencing uses a plurality of probes,
each probe in the plurality of probes includes a nucleic acid sequence that corresponds to the sequence, or a complementary sequence thereof, of a portion of the reference genome represented by a corresponding one or more bins in the plurality of bins.

4. (canceled)

5. The method of claim 1, wherein the second nucleic acid sequencing is a second targeted panel sequencing,
the second targeted panel sequencing uses a plurality of probes, and
each probe in the plurality of probes includes a nucleic acid sequence that corresponds to the sequence, or a complementary sequence thereof, of an allele in the plurality of alleles.

6. The method of claim 5, wherein:
a respective probe in the plurality of probes maps to a portion of the reference genome but has a respective nucleic acid sequence that varies with respect to the portion of the reference genome by one or more transitions, and
each respective transition in the one or more transitions occurs at a respective un-methylated CpG dinucleotide site in the respective portion of the reference genome.

7-9. (canceled)

10. The method of claim 3, wherein deriving the plurality of bin values further comprises using the first plurality of sequence reads to determine a respective number of cell-free nucleic acids represented by the plurality of sequence reads that map to each respective bin in the plurality of bins.

11. (canceled)

12. The method of claim 1, wherein each bin in the plurality of bins comprises at least 100 nucleic acid residues, at least 500 nucleic acid residues, at least 1000 nucleic acid residues, at least 2500 nucleic acid residues, at least 5000 nucleic acid residues, at least 10,000 nucleic acid residues, at least 25,000 nucleic acid residues, at least 50,000 nucleic acid residues, at least 100,000 nucleic acid residues, at least 250,000 nucleic acid residues, or at least at least 500,000 nucleic acid residues.

13. (canceled)

14. The method of claim 1, the plurality of features are applied to the reference model, and the method further comprises determining the plurality of features from the plurality of copy number values by applying a dimensionality reduction method to the plurality of bin values thereby identifying all or a subset of the plurality of features in the form of a plurality of dimension reduction components.

15. The method of claim 1, further comprising deriving the plurality of allele frequencies by using the second plurality of sequence reads to identify support for an allele, and determining an observed frequency of the allele for the variant in the variant set, wherein each observed frequency corresponds to a respective allele frequency in the plurality of allele frequencies.

16-17. (canceled)

18. The method of claim 15, wherein the variant set comprises at least 30 variants, at least 40 variants, at least 50 variants, at least 60 variants, at least 70 variants, at least 80 variants, at least 90 variants, at least 100 variants, at least 200 variants, at least 300 variants, at least 400 variants, at least 500 variants, at least 600 variants, at least 700 variants, at least 800 variants, at least 900 variants, at least 1000 variants, at least 200 variants, at least 3000 variants, at least 400 variants, at least 5000 variants, at least 6000 variants, at least 7000 variants, at least 8000 variants, at least 9000 variants, at least 10,000 variants, at least 20,000 variants, at least 30,000 variants, at least 40,000 variants, at least 50,000 variants, at least 60,000 variants, at least 70,000 variants, at least 80,000 variants, at least 90,000 variants, or at least 100,000 variants.

19. (canceled)

20. The method of claim 1, wherein:
the first plurality of sequence reads provides an average coverage of between 20× and 70,000× across the plurality of bins, and
the second plurality of sequence reads provides an average coverage of between 1,000× and 70,000× across the plurality of bins.

21-23. (canceled)

24. The method of claim 1, wherein the first biological sample and the second biological sample comprise one or a combination selected from the group consisting of blood, whole blood, plasma, serum, urine, cerebrospinal fluid, fecal, saliva, tears, pleural fluid, pericardial fluid, and peritoneal fluid of the subject.

25-30. (canceled)

31. The method of claim 3, wherein each probe in the plurality of probes includes a respective nucleic acid sequence that is complementary or substantially complementary to the reference genome, or a portion thereof, as represented by a bin in the plurality of bins, with the exception that the probe includes an adenine to complement a thymine corresponding to a methylated or unmethylated cytosine in a selected cell-free nucleic acid.

32-33. (canceled)

34. The method of claim 1, wherein:
the first nucleic acid sequencing is methylation sequencing, and
each respective bin value in the first plurality of bin values is a count of a number of cell-free-nucleic acids represented by the first plurality of sequence reads that map to a corresponding bin in the plurality of bins after application of one or more filter conditions.

35. The method of claim 34, wherein:
the methylation sequencing produces a corresponding methylation pattern for each respective cell-free nucleic acid in the first plurality of cell-free nucleic acids, and
a filter condition in the one or more filter conditions is application of a p-value threshold to the corresponding methylation pattern, wherein the p-value threshold is representative of how frequently a methylation pattern is observed in a cohort of non-cancer subjects, and wherein the p-value threshold is below about 0.01.

36-42. (canceled)

43. The method of claim 34, wherein:
the methylation sequencing produces a corresponding methylation pattern for each respective cell-free nucleic acid in the first plurality of cell-free nucleic acids, and
a filter condition in the one or more filter conditions is a requirement that the respective cell-free nucleic acid have a length of less than a threshold number of base pairs.

44-48. (canceled)

49. The method of claim 1, wherein the reference model is a multivariate logistic regression, a neural network, a convolutional neural network, a support vector machine, a decision tree, a regression algorithm, or a supervised clustering model.

50. The method of claim 1, wherein each allele in the plurality of alleles is a single nucleotide variant associated with a predetermined genomic location, an insertion mutation associated with a predetermined genomic location, a deletion mutation associated with a predetermined genomic location, a somatic copy number alteration, a nucleic acid rearrangement associated with a predetermined genomic locus, or an aberrant methylation pattern associated with a predetermined genomic location.

51-54. (canceled)

55. The method of claim 1, the method further comprising:
e) repeating the a) obtaining, b) determining, c) obtaining, and d) applying at each respective time point in a plurality of time points across an epoch, thereby obtaining a corresponding tumor fraction, in a plurality of tumor fractions, for the subject at each respective time point; and
f) using the plurality of tumor fractions to determine a state or progression of a disease condition in the subject during the epoch in the form of an increase or decrease of the first tumor fraction over the epoch.

56. The method of claim 55, wherein the epoch is a period of months and each time point in the plurality of time points is a different time point in the period of months.

57-61. (canceled)

62. The method of claim 55, the method further comprising changing a diagnosis, prognosis, or treatment of the subject when the first tumor fraction of the subject is observed to change by a threshold amount across the epoch.

63-66. (canceled)

67. A non-transitory computer readable storage medium storing at least one program for determining a tumor fraction for a subject of a species, the at least one program configured for execution by a computer, the at least one program comprising instructions for:
a) obtaining, in electronic form, a first dataset that comprises a plurality of bin values, each respective bin value in the plurality of bin values being for a corresponding bin in a plurality of bins, wherein:
each respective bin in the plurality of bins represents a corresponding region of a reference genome of the species, and
the plurality of bin values is derived from alignment of a first plurality of sequence reads, determined by a first nucleic acid sequencing of a first plurality of cell-free nucleic acids in a first biological sample, to a reference genome of the species, wherein the first biological sample comprises a liquid sample of the subject and the first plurality of cell-free nucleic acids comprises at least 1000 cell-free nucleic acids;
b) determining a plurality of copy number values at least in part from the plurality of bins values;
c) obtaining, in electronic form, a second dataset that comprises a plurality of allele frequencies for a plurality of alleles, wherein:
the plurality of allele frequencies is derived from alignment of a second plurality of sequence reads, determined by a second nucleic acid sequencing of a second plurality of cell-free nucleic acids in a second biological sample, to the reference genome, wherein the second biological sample comprises a liquid sample of the subject and the second plurality of cell-free nucleic acids comprises at least 1000 cell-free nucleic acids; and
d) applying, to a reference model, at least the plurality of copy number values and the plurality of allele frequencies, or a plurality of features derived therefrom, thereby determining the tumor fraction of the subject.

68. A computing system, comprising:
at least one processor;
memory storing at least program to be executed by the at least one processor;
the at least one program comprising instructions for determining a tumor fraction for a subject of a species by a method comprising:
a) obtaining, in electronic form, a first dataset that comprises a plurality of bin values, each respective bin value in the plurality of bin values being for a corresponding bin in a plurality of bins, wherein:
each respective bin in the plurality of bins represents a corresponding region of a reference genome of the species, and
the plurality of bin values is derived from alignment of a first plurality of sequence reads, determined by a first nucleic acid sequencing of a first plurality of cell-free nucleic acids in a first biological sample, to a reference genome of the species, wherein the first biological sample comprises a liquid sample of the subject and the first plurality of cell-free nucleic acids comprises at least 1000 cell-free nucleic acids;
b) determining a plurality of copy number values at least in part from the plurality of bins values;
c) obtaining, in electronic form, a second dataset that comprises a plurality of allele frequencies for a plurality of alleles, wherein:
the plurality of allele frequencies is derived from alignment of a second plurality of sequence reads, determined by a second nucleic acid sequencing of a second plurality of cell-free nucleic acids in a second biological sample, to the reference genome, wherein the second biological sample comprises a liquid sample of the subject and the second plurality of cell-free nucleic acids comprises at least 1000 cell-free nucleic acids; and
d) applying, to a reference model, at least the plurality of copy number values and the plurality of allele frequencies, or a plurality of features derived therefrom, thereby determining the tumor fraction of the subject.

69. A method of training a reference model to determine a tumor fraction of a test subject, the method comprising:
at a computer system comprising at least one processor and a memory storing at least one program for execution by the at least one processor, the at least one program comprising instructions for:
a) obtaining a training dataset, in electronic form, that comprises, for each respective reference subject in a plurality of reference subjects, (i) a corresponding plurality of bin values, each respective bin value in the corresponding plurality of bin values being for a corresponding bin in a plurality of bins, (ii) a corresponding plurality of allele frequencies for a corresponding plurality of alleles, and (iii) a corresponding tumor fraction value for the respective reference subject, wherein:
each respective bin in the plurality of bins represents a corresponding region of a reference genome of the species,
each corresponding plurality of bin values is derived from alignment of a corresponding first plurality of sequence reads, determined by a corresponding first nucleic acid sequencing of a corresponding first plurality of cell-free nucleic acids in a corresponding first biological sample, to a reference genome of the species, wherein the first biological sample comprises a liquid sample of a respective reference subject in the plurality of reference subjects and the corresponding first plurality of cell-free nucleic acids comprises at least 1000 cell-free nucleic acids,
each corresponding plurality of allele frequencies is derived from alignment of a corresponding second plurality of sequence reads, determined by a corresponding second nucleic acid sequencing of a corresponding second plurality of cell-free nucleic acids in a second biological sample, to the reference genome, wherein the corresponding second biological sample comprises a liquid sample of a respective reference subject in the plurality of reference subjects and the corresponding second plurality of cell-free nucleic acids comprises at least 1000 cell-free nucleic acids;
b) determining, for each respective reference subject in the plurality of reference subjects, a respective plurality of copy number values at least in part from the corresponding plurality of bins values for the respective reference subject; and
c) obtaining the reference model using at least (i) the respective plurality of copy number values, (ii) the respective plurality of allele frequencies, or a respective plurality of features derived from (i) and (ii), and (iii) the tumor fraction value of each respective reference subject in the plurality of reference subjects.

70-97. (canceled)
